(19) United States
(12) Patent Application Publication
(10) Pub. No.: US 2014/0287937 A1
(43) Pub. Date: Sep. 25, 2014

(54) METHODS FOR ASSESSING CANCER

(71) Applicant: TOMA Biosciences, Inc., Foster City, CA (US)

(72) Inventors: Austin So, Pleasanton, CA (US); Michael Y. LUCERO, South San Francisco, CA (US)

(73) Assignee: TOMA Biosciences, Inc., Foster City, CA (US)

(21) Appl. No.: 14/187,041

(22) Filed: Feb. 21, 2014

Related U.S. Application Data
(60) Provisional application No. 61/767,718, filed on Feb. 21, 2013.
(60) Provisional application No. 61/769,683, filed on Feb. 26, 2013.
(60) Provisional application No. 61/777,702, filed on Mar. 12, 2013.
(60) Provisional application No. 61/780,578, filed on Mar. 13, 2013.

(51) Int. Cl. C12Q 1/68

(52) U.S. Cl. CPC C12Q 1/6827; C12Q 1/6874
USPC 506/7; 435/6.12; 435/6.11

(57) ABSTRACT

Provided herein are methods for assessing cancer, comprising analysis of sequence data from a set of cancer-related genes in a tumor sample from a subject, followed by monitoring of a subset of the set in circulating tumor-associated DNA in a fluid sample from the subject. Also provided are kits and systems for practicing any of them methods of the invention.

### CROSS-REFERENCE

This application claims the benefit of U.S. Provisional Application No. 61/767,718 filed Feb. 21, 2013, U.S. Provisional Application No. 61/769,683, filed Feb. 26, 2013, U.S. Provisional Application No. 61/777,702, filed Mar. 12, 2013, and U.S. Provisional Application No. 61/780,578, filed Mar. 13, 2013, which applications are incorporated herein by reference.

### BACKGROUND OF THE INVENTION

Cancer poses serious challenges for modern medicine. In 2007, it has been estimated that cancer caused about 13% of all human deaths worldwide (7.9 million). Cancer encompasses a broad group of various diseases, generally involving unregulated cell growth. In cancer, cells can divide and grow uncontrollably, can form malignant tumors, and can invade nearby parts of the body. Cancer can also spread to more distant parts of the body, for example, via the lymphatic system or bloodstream. There are over 200 different known cancers that afflict humans. Many cancers are associated with mutations, for example, mutations in cancer-related genes. The mutational status of a cancer can vary widely from one individual subject to another, and even from one tumor cell to another tumor cell in the same subject. Knowledge of these mutations can aid in the selection of cancer therapy, and can also aid in informing disease prognosis and/or disease status. A tumor biopsy can be sequenced to provide information on mutational status of cancer-related genes; however, procedures for tumor biopsies can be surgically invasive and costly to a patient. Furthermore, reliance on a tumor biopsy is of limited utility for monitoring cancer status of a subject if the subject has tumor cells that are difficult to biopsy (e.g., if a tumor is small).

The discovery that cell-free DNA floating in blood plasma and serum can harbor tumor-associated mutations opened up the possibility that analysis of cell-free DNA could aid in cancer diagnosis. However, analysis of cell-free tumor DNA, as currently practiced utilizes untargeted sequencing or complicated PCR amplification (e.g., on magnetic beads), resulting in high costs due to expensive reagents and systems.

The detection and/or measurement of mutations is widely practiced in the life sciences. For example, mutations such as single nucleotide polymorphisms (SNPs) are associated with a number of diseases, including, e.g., cancer, neurodegenerative diseases, infectious diseases, autoimmune diseases, anemia, and cystic fibrosis. Current methods for detecting mutations generally involve the amplification of target polynucleotides. For example, target-specific primers can selectively amplify regions suspected of harboring a mutation, and the resulting amplicons can be sequenced to interrogate the mutation. By way of other example, assays may utilize intercalating dyes that fluoresce in the presence of double stranded DNA (dsDNA), or may utilize Taqman probes designed to hybridize specifically to a particular polynucleotide sequence. These assays generally suffer from poor specificity and/or sensitivity, particularly for mutations affecting small nucleotide sequences (e.g., SNPs or small insertions/deletions).

### SUMMARY OF THE INVENTION

Aspects of the invention relate to methods and kits for assessing cancer. Other aspects of this invention relate to methods and kits for preparing a sample library for sequencing.

In some instances, the invention provides a method of assessing cancer, comprising: (a) determining the presence, absence, and/or amount of each of a subset of genes in a sample derived from a sample from a subject, wherein the subset is determined by (i) performing targeted sequencing on a set of genes on a solid tissue sample from the subject wherein the solid tissue sample is known or suspected of comprising cancerous tissue; (ii) determining a profile of genetic abnormalities for the set of genes based on the sequencing; and (iii) selecting a subset of 2, 3, or 4 genes of the set of genes based on the profile for the set, wherein the subset is specific to the individual; and (b) from the results of step (a) determining the status of the cancer in the subject.

The method can comprise (a) determining the presence, absence, and/or amount of each of a subset of genes in a sample derived from a fluid sample in a subject, wherein the subset is determined by (i) performing targeted sequencing on a set of genes from an unfixed solid tissue sample from the subject wherein the solid tissue sample is known or suspected of comprising cancerous tissue; (ii) determining a profile of genetic abnormalities for the set of genes based on the sequencing; and (iii) selecting a subset of the set of genes based on the profile for the set, wherein the subset is specific to the individual; and (b) from the results of step (a) determining the status of the cancer in the subject.

In a related embodiment, the method comprises (a) determining the presence, absence, and/or amount of each of a subset of genes in a sample derived from a fluid sample in a subject, wherein the subset is determined by (i) performing targeted sequencing on a set of genes from a bodily fluid sample from the subject wherein the bodily fluid sample is known or suspected of comprising tumor-derived nucleic acid; (ii) determining a profile of genetic abnormalities for the set of genes based on the sequencing; and (iii) selecting a subset of the set of genes based on the profile for the set, wherein the subset is specific to the individual; and (b) from the results of step (a) determining the status of the cancer in the subject.

In practicing any of the methods described herein, the set of genes comprises at least 10, 20, 30, 40, 50, 60, 70, 80, 90, 100, 150, 200, 300, 400, 500, 600, 700, 800, 900, or 1000 genes.

The set of genes can be selected from the group consisting of: ABCA1, BRAF, CHD5, EP300, FLT1, ITPA, MYC, P1K3R1, SKP2, TP53, ABCA7, BRCA1, CHEK1, EPHA3, FLT3, JAK1, MYCL1, PIK3R2, SLC19A1, TP73, ABCB1, BRCA2, CHEK2, EPHA5, FLT4, JAK2, MYCN, PKHD1, SLC1A6, TPM3, ABCC2, BRIP1, CLTC, EPHA6, FN1, JAK3, MYH2, PLCB1, SLC22A2, TPMT, ABCC3, BUB1B, COL1A1, EPHA7, FOS, JUN, MYH9, PLCG1, SLCO1B3, TPO, ABCC4, Clorf144, COPS5, EPHA8, FOXO1, KBTBD11, NAV3, PLCG2, SMAD2, TPR, ABCG2, CABLES1, CREB1, EPHB1, FOXO3, KDM6A, NBN, PML, SMAD3, TR10, ABL1, CACNA2D1, CREBBP, EPHB4, FOXP4, KDR, NCOA2, PMS2, SMAD4, TRRAP, ABL2, CAMKV, CRKL, EPHB6, GAB1, KIT, NEK11, PPARG, SMARCA4, TSC1, ACVR1B, CARD11, CRLF2, EPO, GATA1, KLF6, NF1, PPARGC1A, SMARCB1, TSC2, ACVR2A, CARM1, CSF1R, ERBB2, GLI1, KLHDC4, NF2, PPP1R3A, SMO, TTK, ADCY9, CAV1, CSMD3, ERBB3, GLI3, KRAS, NKX2-1, PPP2R1A, SOCS1, TYK2, AGAP2, CBFA2T3, CSNK1G2, ERBB4, GNA11, LMO2, NOS2, PPP2R1B, SOD2, TYMS, AKT1, CBL, CTNNA1, ERCC1, GNAQ, LRP1B, NOS3, PRKAA2, SOS1, UGT1A1, AKT2, CCND1, CTNNA2, ERCC2, GNAS, LRP2, NOTCH1, PRKCA, SOX10, UMPS, AKT3, CCND2, CTNNB1, ERCC3, GPR124, LRP6, NOTCH2, PRKCZ, SOX2, USP9X, ALK, CCND3, CYFIP1, ERCC4, GPR133, LTK, NOTCH3, PRKDC, SP1, VEGF, ANAPC5, CCNE1, CYLD, ERCC5, GRB2, MAN1B1, NPM1, PTCH1, SPRY2, VEGFA, APC, CD40LG, CYP19A1, ERCC6, GSK3B, MAP2K1, NQO1, PTCH2, SRC, VHL, APC2, CD44, CYP1B1, ERG, GSTP1, MAP2K2, NR3Cl, PTEN, ST6GAL2, WRN, AR, CD79A, CYP2C19, ERN2, GUCY1A2, MAP2K4, NRAS, PTGS2, STAT1, WT1, ARAF, CD79B, CYP2C8, ESR1, HDAC1, MAP2K7, NRP2, PTPN11, STAT3, XPA, ARFRP1, CDC42, CYP2D6, ESR2, HDAC2, MAP3K1, NTRK1, PTPRB, STK11, XPC, ARID1A, CDC42BPB, CYP3A4, ETV4, HGF, MAPK1, NTRK2, PTPRD, SUFU, ZFY, ATM, CDC73, CYP3A5, EWSR1, HIF1A, MAPK3, NTRK3, RAD50, SULT1A1, ZNF521, ATP5A1, CDH1, DACH2, EXT1, HM13, MAPK8, OMA1, RAD51, SUZ12, ATR, CDH10, DCC, EZH2, HMGA1, MARK3, OR10R2, RAFT, TAF1, AURKA, CDH2, DCLK3, FANCA, HNF1A, MCL1, PAK3, RARA, TBX22, AURKB, CDH2O, DDB2, FANCD2, HOXA3, MDM2, PARP1, RB1, TCF12, BAI3, CDH5, DDR2, FANCE, HOXA9, MDM4, PAX5, REM1, TCF3, BAP1, CDK2, DGKB, FANCF, HRAS, MECOM, PCDH15, RET, TCF4, BARD1, CDK4, DGKZ, FAS, HSP90AA1, MEN1, PCDH18, RICTOR, TEK, BAX, CDK6, DIRAS3, FBXW7, IDH1, MET, PCNA, RIPK1, TEP1, BCL11A, CDK7, DLG3, FCGR3A, IDH2, MITF, PDGFA, ROR1, TERT, BCL2, CDK8, DLL1, FES, IFNG, MLH1, PDGFB, ROR2, TET2, BCL2A1, CDKN1A, DNMT1, FGFR1, IGF1R, MLL, PDGFRA, ROS1, TGFBR2, BCL2L1, CDKN1B, DNMT3A, FGFR2, IGF2R, MLL3, PDGFRB, RPS6KA2, THBS1, BCL2L2, CDKN2A, DNMT3B, FGFR3, IKBKE, MPL, PDZRN3, RPTOR, TNFAIP3, BCL3, CDKN2B, DOT1L, FGFR4, IKZF1, MRE11A, PHLPP2, RSPO2, TNKS, BCL6, CDKN2C, DPYD, FH, IL2RG, MSH2, PIK3C3, RSPO3, TNKS2, BCR, CDKN2D, E2F1, FHOD3, INHBA, MSH6, PIK3CA, RUNX1, TNNI3K, BIRC5, CDX2, EED, FIGF, INSR, MTHFR, PIK3CB, SDHB, TNR, BIRC6, CEBPA, EGF, FLG2, IRS1, MTOR, PIK3CD, SF3B1, TOP1, BLM, CERK, EGFR, FLNC, IRS2, MUTYH, PIK3CG, SHC1, and TOP2A.

The sample can be selected from the group consisting of: blood, serum, plasma, urine, sweat, tears, saliva, sputum, components thereof or any combination thereof.

Steps (a) and (b) can be performed at a plurality of time points to monitor the status of the cancer over time. One time point can be prior to a first administration of a cancer therapy and a subsequent time point can be subsequent to a first administration.

The method can further comprise generating a report communicating the profile of genetic abnormalities for the set of genes and communicating the report to a caregiver.

The report can comprise a list of one or more therapeutic candidates based on the profile.

The report can be generated within two weeks from collection of the solid tissue sample. In some instances, the report is generated within 1 week from collection of the solid tissue sample.

In some embodiments, the report comprises copy number alterations of the set of genes.

In some embodiments, the report comprises a description of a therapeutic agent targeting a abnormality.

The methods described herein further comprises generating a report communicating the profile of the subset of genes at each of the plurality of time points.

In some embodiments of any of the methods herein, the determining comprises the step of diluting nucleic acid molecules from the sample into discrete reaction volumes, wherein the discrete reaction volumes contain on average less than 10, 5, 4, 3, 2, or 1 nucleic acid molecule from the sample.

The discrete reaction volumes can be droplets in an emulsion. The discrete reaction volumes further comprise primers for allelic discrimination of the genetic abnormalities in the subset of genes

Determining the status comprises quantifying the number of nucleic acids harboring the genetic abnormalities in the subset of genes.

The step of targeted sequencing can comprise preparing a DNA library from the solid tissue sample in less than 8, 7, 6, 5, or 4 hours.

The preparing may not require exponential PCR amplification prior to sequencing of the library.

The preparing can comprise a linear amplification step. In some embodiments the preparing does not require amplification.

In some embodiments, the step of targeted sequencing comprises (a) contacting a single-stranded DNA fragment from the solid tissue sample with a target-specific oligonucleotide comprising (i) a region specific for a region of a cancer-related gene and (ii) an adaptor sequence specific for coupling to a sequencing platform; (b) performing a hybridization reaction to join the target specific oligonucleotides to a single-stranded DNA fragment containing a region of complementarity to the target-specific oligonucleotide; (c) performing an extension reaction to create an extension product comprising the region and comprising the adaptor; and (d) sequencing the extension product.

Contacting can occur with the target-specific oligonucleotide attached to a sequencing platform.

Contacting can occur with the target-specific oligonucleotide free in a solution.

In some aspects, the present invention provides methods and kits for the sensitive detection of a mutation in a target polynucleotide.

The invention also provides an oligonucleotide primer, comprising a probe-binding region and a template binding region. In some embodiments, the template binding region is at least 50% complementary to a template nucleic acid suspected of harboring a mutation. In some embodiments, a portion of the template binding region at least partially overlays a locus of the suspected mutation. In some embodiments, the oligonucleotide primer upon hybridization to the template nucleic acid is extendable by a polymerase if the mutation is present but is not extendable by the polymerase if the mutation is not present.

In some embodiments, the template binding region comprises a 3′ terminal region that overlays the mutation locus. In some embodiments, the 3′ terminal region that overlays the mutation locus comprises 1, 2, 3, 4, 5, or more than 5 bases of the 3′-end of the template binding region.

In some embodiments, the mutation is a single nucleotide polymorphism (SNP).

In particular embodiments, the 3′ terminal region comprises a base that overlays the SNP locus. In some embodiments, the base is complementary to a mutant allele of the SNP locus. In some embodiments, the base is complementary to a wild-type allele of the SNP locus.

In some embodiments, the probe-binding region does not hybridize to any genomic sequence from the subject.

In some embodiments, the polymerase is a DNA polymerase lacking 3′ to 5′ exonuclease activity.

The invention also provides a kit comprising: (a) an oligonucleotide primer, wherein the oligonucleotide primer comprises (i) a probe-binding region and a template binding region that is at least 70% complementary to a template nucleic acid suspected of harboring a mutation, wherein a portion of the template binding region at least partially overlays locus of the suspected mutation, wherein the oligonucleotide primer upon hybridization to the template nucleic acid is extendable by a polymerase if the mutation is present but is not extendable by the polymerase if the mutation is not present; and (b) instructions for use.

In some embodiments, the mutation is a single nucleotide polymorphism (SNP).

In some embodiments, the template binding region comprises a 3′ terminal base that overlays the SNP locus.

In some embodiments, the 3′ terminal base is complementary to a mutant allele of the SNP locus.

In some embodiments, the 3′ terminal base is complementary to a wild-type allele of the SNP locus.

In some embodiments, the probe-binding region does not hybridize to any genomic sequence from the subject.

In some embodiments, the kit further comprises a reporter probe that is at least 70% complementary to the probe binding region.

In some embodiments, the reporter probe comprises a detectable moiety and a quencher moiety, wherein the quencher moiety suppresses detection of the detectable moiety when the reporter probe is intact.

In some embodiments, the kit further comprises a reverse primer that is at least 70% complementary to a reverse complement sequence downstream of the locus.

In some embodiments, the kit further comprises a polymerase.

In some embodiments, the polymerase is a thermostable polymerase having a 5′ to 3′ exonuclease activity and not having a 3′ to 5′ exonuclease activity.

In some embodiments, the kit further comprises (i) one or more alternative oligonucleotide primers, wherein the one or more alternative oligonucleotide primers each comprises a distinct probe binding region and a template binding region that is at least 70% complementary to the template nucleic acid, wherein a portion of the template binding region at least partially overlays the locus, wherein the alternative oligonucleotide primer upon hybridization to the template nucleic acid is extendable by a polymerase if an alternative allele is present but is not extendable by the polymerase if the alternative allele is not present.

In some embodiments, the kit further comprises one or more alternative reporter probes, wherein each of the alternative reporter probes is at least 70% complementary to one of the distinct probe binding regions but not to any other probe binding region of the kit.

In some embodiments, each of the alternative reporter probes comprises an alternative detectable moiety and a quencher moiety, wherein each of the detectable moieties of the kit is detectably distinct from any other detectable moiety of the kit.

In some embodiments, a hybridization product consisting of the oligonucleotide primer and reporter probe has a Tm that is at least 10 degrees higher than a Tm of a hybridization product consisting of the oligonucleotide primer and the template nucleic acid.

In another aspect, the invention provides a method of detecting a mutation in a target polynucleotide region, comprising: (a) selectively hybridizing an oligonucleotide primer to the target polynucleotide region, wherein the oligonucleotide primer comprises (i) a probe-binding region, and (ii) a template binding region that is at least 70% complementary to a template nucleic acid suspected of harboring a mutation, wherein a portion of the template binding region at least partially overlays a locus of the suspected mutation, and wherein the oligonucleotide primer upon hybridization to the template nucleic acid is extendable by a polymerase if the mutation is present but is not extendable by the polymerase if the mutation is not present; (b) extending the hybridized oligonucleotide primer to form an extension product; and (c) detecting the extension product, whereby the detecting indicates the presence of the mutation.

In some embodiments, extending comprises extending with a DNA polymerase that does not comprise 3′ to 5′ exonuclease activity.

In some embodiments, detecting comprises selectively hybridizing a reporter probe to the probe binding region.

In some embodiments, the reporter probe comprises a detectable moiety and a quencher moiety, wherein the quencher moiety suppresses detection of the detectable moiety when the reporter probe is intact.

In some embodiments, detecting further comprises separating the detectable moiety from the quencher moiety of the hybridized reporter probe.

In some embodiments, the method further comprises amplifying the extension product with a reverse primer that is capable of hybridizing to a region of the extension product downstream of the locus.

In some embodiments, amplifying comprises amplifying with a DNA polymerase that comprises 5′ to 3′ exonuclease activity.

In some embodiments, the method further comprises selectively hybridizing one or more alternative oligonucleotide primers to the target polynucleotide region, wherein the one or more alternative oligonucleotide primers each comprises a distinct probe binding region and a template binding region that is at least 70% complementary to the template nucleic acid, wherein a portion of the template binding region at least partially overlays the locus, wherein the alternative oligonucleotide primer upon hybridization to the template nucleic acid is extendable by a polymerase if an alternative allele is present but is not extendable by the polymerase if the alternative allele is not present.

In some embodiments, detecting further comprises selectively hybridizing one or more alternative reporter probes to the one or more alternative oligonucleotide primers, wherein each of the alternative reporter probes is at least 70% complementary to one of the distinct probe binding regions but not to any other of the probe binding regions.

In some embodiments, each of the alternative reporter probes comprises an alternative detectable moiety and a quencher moiety, wherein each of the alternative detectable moieties is detectably distinct from any other of the detectable moieties.

In some embodiments, the mutation is a single nucleotide polymorphism (SNP).

In some embodiments, the template binding region comprises a 3′ terminal region comprising a base that overlays the SNP locus.

In some embodiments, wherein the base is complementary to a mutant allele of the SNP locus.

In some embodiments, the base is complementary to a wild-type allele of the SNP locus. In some embodiments, the probe-binding region does not hybridize to the target polynucleotide region.

In some embodiments, a hybridization product of the oligonucleotide primer and reporter probe has a Tm that is at least 10 degrees higher than a Tm of a hybridization product between the oligonucleotide primer and target polynucleotide.

In some embodiments, a concentration of the reporter probe is at least 10× a concentration of the forward primer.

In some embodiments, the nucleic acid sample is subdivided into a plurality of discrete reaction volumes prior to steps b-c.

In some embodiments, the method further comprises detection of the detectable moiety in each of the reaction volumes.

In some embodiments, the method further comprises counting a number of the reaction volumes wherein the detectable moiety is detected.

In some embodiments, the nucleic acid sample is subdivided such that the plurality of discrete reaction volumes contain an average of <1 template nucleic acid molecule.

In some embodiments, the method further comprises providing a conclusion and transmitting the conclusion over a network.

The invention also provides a composition comprising (a) an oligonucleotide primer hybridized to a template nucleic acid, wherein the template nucleic acid comprises a wild-type allele at a locus, wherein the 3′ terminal region of the oligonucleotide primer overlays the locus and is not complementary to the wild-type allele; and (b) an intact reporter probe comprising a detectable and quencher moiety, wherein the intact reporter probe is hybridized to the oligonucleotide primer.

The invention also provides a method, comprising: (a) hybridizing a target-selective oligonucleotide (TSO) to a single-stranded DNA (ssDNA) fragment in an ssDNA library to create a hybridization product; and (b) amplifying the hybridization product to create a double stranded extension product, wherein the TSO comprises (i) a sequence that is complementary to a single target region and (ii) a first single-stranded adaptor sequence located at a first end of the TSO but not to both ends of the TSO, and wherein the ssDNA fragment comprises a second single-stranded adaptor sequence but does not comprise the first single-stranded adaptor sequence. In some embodiments, the second single-stranded adaptor sequence is located at a first end of the ssDNA fragment but not at both ends of the ssDNA fragment. In some embodiments, the amplifying comprises linear amplification. In some embodiments, the second single-stranded adaptor sequence is located at a first end of the ssDNA fragment but not at both ends of the ssDNA fragment. In some embodiments, the first end of the ssDNA fragment is a 5′ end. In some embodiments, the first adaptor sequence comprises a barcode sequence. In some embodiments, the first or second adaptor sequence comprises a barcode sequence. In some embodiments, the first end of the TSO is a 5′ end. In some embodiments, the first or second adaptor sequence comprises a sequence that is at least 70% identical to a support-bound oligonucleotide conjugated to a solid support. In some embodiments, the solid support is coupled to a sequencing platform. In some embodiments, the first or second adaptor sequence comprises a binding site for a sequencing primer. In some embodiments, the method further comprises annealing the extension products to the support-bound oligonucleotides. In some embodiments, the method further comprises amplifying the annealed extension products. In some embodiments, the method further comprises sequencing the annealed extension products. In some embodiments, the ssDNA library comprises genomic DNA fragments. In some embodiments, the ssDNA library comprises cDNA fragments.

In some embodiments, the method further comprises removing unhybridized TSOs and unhybridized ssDNA library members. In some embodiments, steps (a) and (b) are performed when the ssDNA library members and the TSOs are free-floating in a solution.

In some embodiments, the single target region flanks a genomic region. In some embodiments, the genomic region comprises a portion of an exon region from a cancer-related gene. In some embodiments, the cancer-related gene is selected from the group consisting of ABCA1, BRAF, CHD5, EP300, FLT1, ITPA, MYC, P1K3R1, SKP2, TP53, ABCA7, BRCA1, CHEK1, EPHA3, FLT3, JAK1, MYCL1, PIK3R2, SLC19A1, TP73, ABCB1, BRCA2, CHEK2, EPHA5, FLT4, JAK2, MYCN, PKHD1, SLC1A6, TPM3, ABCC2, BRIP1, CLTC, EPHA6, FN1, JAK3, MYH2, PLCB1, SLC22A2, TPMT, ABCC3, BUB1B, COL1A1, EPHA7, FOS, JUN, MYH9, PLCG1, SLCO1B3, TPO, ABCC4, Clorf144, COPS5, EPHA8, FOXO1, KBTBD11, NAV3, PLCG2, SMAD2, TPR, ABCG2, CABLES1, CREB1, EPHB1, FOXO3, KDM6A, NBN, PML, SMAD3, TR10, ABL1, CACNA2D1, CREBBP, EPHB4, FOXP4, KDR, NCOA2, PMS2, SMAD4, TRRAP, ABL2, CAMKV, CRKL, EPHB6, GAB1, KIT, NEK11, PPARG, SMARCA4, TSC1, ACVR1B, CARD11, CRLF2, EPO, GATA1, KLF6, NF1, PPARGC1A, SMARCB1, TSC2, ACVR2A, CARM1, CSF1R, ERBB2, GLI1, KLHDC4, NF2, PPP1R3A, SMO, TTK, ADCY9, CAV1, CSMD3, ERBB3, GLI3, KRAS, NKX2-1, PPP2R1A, SOCS1, TYK2, AGAP2, CBFA2T3, CSNK1G2, ERBB4, GNA11, LMO2, NOS2, PPP2R1B, SOD2, TYMS, AKT1, CBL, CTNNA1, ERCC1, GNAQ, LRP1B, NOS3, PRKAA2, SOS1, UGT1A1, AKT2, CCND1, CTNNA2, ERCC2, GNAS, LRP2, NOTCH1, PRKCA, SOX10, UMPS, AKT3, CCND2, CTNNB1, ERCC3, GPR124, LRP6, NOTCH2, PRKCZ, SOX2, USP9X, ALK, CCND3, CYFIP1, ERCC4, GPR133, LTK, NOTCH3, PRKDC, SP1, VEGF, ANAPC5, CCNE1, CYLD, ERCC5, GRB2, MAN1B1, NPM1, PTCH1, SPRy2, VEGFA, APC, CD40LG, CYP19A1, ERCC6, GSK3B, MAP2K1, NQO1, PTCH2, SRC, VHL, APC2, CD44, CYP1B1, ERG, GSTP1, MAP2K2, NR3Cl, PTEN, ST6GAL2, WRN, AR, CD79A, CYP2C19, ERN2, GUCY1A2, MAP2K4, NRAS, PTGS2, STAT1, WT1, ARAF, CD79B, CYP2C8, ESR1, HDAC1, MAP2K7, NRP2, PTPN11, STAT3, XPA, ARFRP1, CDC42, CYP2D6, ESR2, HDAC2, MAP3K1, NTRK1, PTPRB, STK11, XPC, ARID1A, CDC42BPB, CYP3A4, ETV4, HGF, MAPK1, NTRK2, PTPRD, SUFU, ZFY, ATM, CDC73, CYP3A5, EWSR1, HIF1A, MAPK3, NTRK3, RAD50, SULT1A1, ZNF521, ATP5A1, CDH1, DACH2, EXT1, HM13, MAPK8, OMA1, RAD51, SUZ12, ATR, CDH10, DCC, EZH2, HMGA1, MARK3, OR10R2, RAFT, TAF1, AURKA, CDH2, DCLK3, FANCA, HNF1A, MCL1, PAK3, RARA, TBX22, AURKB, CDH2O, DDB2, FANCD2, HOXA3, MDM2, PARP1, RB1, TCF12, BAI3, CDH5, DDR2, FANCE, HOXA9, MDM4, PAX5, REM1, TCF3, BAP1, CDK2, DGKB, FANCF, HRAS, MECOM, PCDH15, RET, TCF4, BARD1, CDK4, DGKZ, FAS, HSP90AA1, MEN1, PCDH18, RICTOR, TEK, BAX, CDK6, DIRAS3, FBXW7, IDH1, MET, PCNA, RIPK1, TEP1, BCL11A, CDK7, DLG3, FCGR3A, IDH2, MITF, PDGFA, ROR1, TERT, BCL2, CDK8, DLL1, FES, IFNG, MLH1, PDGFB, ROR2, TET2, BCL2A1, CDKN1A, DNMT1, FGFR1, IGF1R, MLL, PDGFRA, ROS1, TGFBR2, BCL2L1, CDKN1B, DNMT3A, FGFR2, IGF2R, MLL3, PDGFRB, RPS6KA2, THBS1, BCL2L2, CDKN2A, DNMT3B, FGFR3, IKBKE, MPL, PDZRN3, RPTOR, TNFAIP3, BCL3, CDKN2B, DOT1L, FGFR4, IKZF1, MRE11A, PHLPP2, RSPO2, TNKS, BCL6, CDKN2C, DPYD, FH, IL2RG, MSH2, PIK3C3, RSPO3, TNKS2, BCR, CDKN2D, E2F1, FHOD3, INHBA, MSH6, PIK3CA, RUNX1, TNNI3K, BIRC5, CDX2, EED, FIGF, INSR, MTHFR, PIK3CB, SDHB, TNR, BIRC6, CEBPA, EGF, FLG2, IRS1, MTOR, PIK3CD, SF3B1, TOP1, BLM, CERK, EGFR, FLNC, IRS2, MUTYH, PIK3CG, SHC1, and TOP2A.

The invention also provides a method of preparing a single-stranded DNA library, comprising: (a) denaturing a double stranded DNA fragment into single stranded DNA (ssDNA) fragments; (b) removing 5′ phosphates from the ssDNA fragments; (c) ligating single-stranded primer docking oligonucleotides (pdo's) to 3′ ends of the ssDNA fragments, wherein the pdo's are conjugated to a capture moiety capable of binding to an immobilized capturing reagent; (d) hybridizing primers to the pdo's, wherein the primers comprise a sequence complementary to the adaptor oligonucleotide sequence and comprise a first adaptor sequence that is at least 70% identical to a support-bound oligonucleotide coupled to a sequencing platform; (e) extending the hybridized primers to create duplexes, wherein each duplex comprises an ss fragment and an extended primer strand; immobilizing the duplexes to the immobilized capturing reagent; (f) denaturing the double-stranded extension product, wherein the denaturing results in release of the extended primer strands from the immobilized capturing reagent and retention of the ssDNA fragments on the immobilized capturing reagent; and (g) collecting the extended primer strands, wherein the extended primer strands comprise the ss DNA library. In some embodiments, step (c) results in ligation of at least 50% of the ssDNA fragments to the pdo's. In some embodiments, the ligating is performed using an ATP-dependent ligase. In some embodiments, the ATP-dependent ligase is an RNA ligase. In some embodiments, the RNA ligase is CircLigase or CircLigase II. In some embodiments, the pdo's are adenylated. In some embodiments, the extending is performed using a proofreading DNA polymerase.

The invention also provides a method of preparing a single-stranded DNA library, comprising: denaturing a double stranded DNA fragment into single stranded DNA (ssDNA) fragments; ligating a first single-stranded adaptor sequence to a first end of the ssDNA fragments; and ligating a second single-stranded adaptor sequence to a second end of the ssDNA fragments.

The invention also provides a kit, comprising: a primer docking oligonucleotide (pdo), wherein the pdo is conjugated to a capture moiety capable of binding to an immobilized capturing reagent; a primer, wherein the primer comprises a sequence that is at least 70% complementary to the pdo sequence and further comprises a first adaptor sequence that is at least 70% identical to a first support-bound oligonucleotide coupled to a sequencing platform; and instructions for use. In some embodiments, the kit further comprises an ATP-dependent ligase. In some embodiments, the ATP-dependent ligase is an RNA ligase.

In some embodiments, the RNA ligase is CircLigase or CircLigase II. In some embodiments, the kit further comprises a proofreading DNA polymerase. In some embodiments, the kit further comprises the immobilized capturing reagent.

In some embodiments, the first adaptor sequence comprises a sequence that is at least 70% complementary to a first sequencing primer. In some embodiments, the first adaptor sequence comprises a barcode sequence. In some embodiments, the kit further comprises a target-selective oligonucleotide (TSO). In some embodiments, the TSO further comprises a second adaptor sequence located at a first end of the TSO but not a second end of the TSO. In some embodiments, the first end of the TSO is a 5′ end. In some embodiments, the second adaptor sequence comprises a sequence that is at least 70% identical to a second support-bound oligonucleotide coupled to a sequencing platform. In some embodiments, the second adaptor sequence comprises a binding site for a sequencing primer.

The invention also provides a kit, comprising: a first adaptor oligonucleotide, wherein the first adaptor comprises a sequence that is at least 70% complementary to a first support-bound oligonucleotide coupled to a sequencing platform; a second adaptor oligonucleotide, wherein the second adaptor comprises a sequence that is distinct from the first adaptor oligonucleotide; an RNA ligase; and instructions for use. In some embodiments, the second adaptor comprises a sequence that is at least 70% complementary to a sequencing primer. In some embodiments, the second adaptor comprises a sequence that is at least 70% complementary to a second support-bound oligonucleotide coupled to a sequencing platform. In some embodiments, the first adaptor comprises a sequence that is at least 70% complementary to a sequencing primer. In some embodiments, one of the first or second adaptor comprises a barcode sequence. In some embodiments, the first adaptor comprises a 3′ terminal blocking group that prevents the formation of a covalent bond between the 3′ terminal base and another nucleotide. In some embodiments, the 3′ terminal blocking group is dideoxy-dNTP, biotin, or other possible blocking group. In some embodiments, the first adaptor comprises a 5′polyadenylation sequence. In some embodiments, the RNA ligase is truncated or mutated ligase 2 from T4 or Mth. In some embodiments, the kit further comprises a second RNA ligase. In some embodiments, the second RNA ligase is CircLigase or CircLigase II.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts an exemplary workflow of a method for assessing cancer in a subject.

FIG. 2 depicts an exemplary workflow of a method for sequencing a tumor cell and a normal cell in a subject.

FIG. 3 depicts an exemplary embodiment of a method for collecting a tumor and blood sample from a subject.

FIG. 4 depicts an exemplary workflow for a method of preparing a DNA library from a tumor sample of a subject.

FIG. 5 depicts an exemplary embodiment of a method of preparing a DNA library from a tumor sample of a subject.

FIG. 6 depicts an exemplary embodiment of a method of assessing tumor-specific mutations in cell-free DNA from a blood sample of a subject FIGS. 7A and 7B depict an exemplary workflow for allele detection in a sample.

FIG. 8 depicts an exemplary embodiment of a subject-specific report of tumor-specific mutations in a subject.

FIG. 9 depicts a workflow used for assessment of a colon cancer subject.

FIG. 10 depicts results from a validation assay for detecting alleles of a mutation found in the tumor of the colon cancer patient.

FIG. 11 depicts a system for software facilitation.

FIG. 12 depicts results from a validation assay for detecting alleles of a mutation found in the tumor of the colon cancer patient.

### DETAILED DESCRIPTION OF THE INVENTION

The practice of the present invention will employ, unless otherwise indicated, conventional techniques of molecular biology, microbiology and recombinant DNA techniques, which are within the skill of the art. Such techniques are explained fully in the literature. See, e.g., Sambrook, Fritsch & Maniatis, Molecular Cloning: A Laboratory Manual, Second Edition (1989); Oligonucleotide Synthesis (M. J. Gait, ed., 1984); Nucleic Acid Hybridization (B. D. Hames & S. J. Higgins, eds., 1984); A Practical Guide to Molecular Cloning (B. Perbal, 1984); and a series, Methods in Enzymology (Academic Press, Inc.). All patents, patent applications, and publications mentioned herein, both supra and infra, are hereby incorporated by reference.

### DEFINITIONS

As used in the specification and claims, the singular forms “a”, “an” and “the” can include plural references unless the context clearly dictates otherwise. For example, the term “a cell” can include a plurality of cells, including mixtures thereof.

The term “subject”, as used herein, generally refers to a biological entity containing expressed genetic materials. The biological entity can be a plant, animal, or microorganism, including, e.g., bacteria, viruses, fungi, and protozoa. The subject can be tissues, cells and their progeny of a biological entity obtained in vivo or cultured in vitro. The subject can be a mammal. The mammal can be a human. The human may be diagnosed or suspected of being at high risk for a disease. The disease can be cancer. The human may not be diagnosed or suspected of being at high risk for a disease.

As used herein, a “sample” or “nucleic acid sample” can refer to any substance containing or presumed to contain nucleic acid. The sample can be a biological sample obtained from a subject. The nucleic acids can be RNA, DNA, e.g., genomic DNA, mitochondrial DNA, viral DNA, synthetic DNA, or cDNA reverse transcribed from RNA. The nucleic acids in a nucleic acid sample generally serve as templates for extension of a hybridized primer. In some embodiments, the biological sample is a liquid sample. The liquid sample can be whole blood, plasma, serum, ascites, cerebrospinal fluid, sweat, urine, tears, saliva, buccal sample, cavity rinse, or organ rinse. The liquid sample can be an essentially cell-free liquid sample (e.g., plasma, serum, sweat, urine, tears, etc). In other embodiments, the biological sample is a solid biological sample, e.g., feces or tissue biopsy, e.g., a tumor biopsy. A sample can also comprise in vitro cell culture constituents (including but not limited to conditioned medium resulting from the growth of cells in cell culture medium, recombinant cells and cell components).

“Nucleotides” can be biological molecules that can form nucleic acids. Nucleotides can have moieties that contain not only the known purine and pyrimidine bases, but also other heterocyclic bases that have been modified. Such modifications include methylated purines or pyrimidines, acylated purines or pyrimidines, alkylated riboses, or other heterocycles. In addition, the term “nucleotide” includes those moieties that contain hapten, biotin, or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the like.

“Nucleotides” can also include locked nucleic acids (LNA) or bridged nucleic acids (BNA). BNA and LNA generally refer to modified ribonucleotides wherein the ribose moiety is modified with a bridge connecting the 2′ oxygen and 4′ carbon. Generally, the bridge “locks” the ribose in the 3′-endo (North) conformation, which is often found in the A-form duplexes. The term “locked nucleic acid” (LNA) generally refers to a class of BNAs, where the ribose ring is “locked” with a methylene bridge connecting the 2′-O atom with the 4′-C atom. LNA nucleosides containing the six common nucleobases (T, C, G, A, U and mC) that appear in DNA and RNA are able to form base-pairs with their complementary nucleosides according to the standard Watson-Crick base pairing rules. Accordingly, BNA and LNA nucleotides can be mixed with DNA or RNA bases in an oligonucleotide whenever desired. The locked ribose conformation enhances base stacking and backbone pre-organization. Base stacking and backbone pre-organization can give rise to an increased thermal stability (e.g., increased Tm) and discriminative power of duplexes. LNA can discriminate single base mismatches under conditions not possible with other nucleic acids. Locked nucleic acid is disclosed for example in WO 99/14226. Nucleotides can also include modified nucleotides as described in European Patent Application No. EP1995330.

The terms “polynucleotides”, “nucleic acid”, “nucleotides” and “oligonucleotides” can be used interchangeably. They can refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “target polynucleotide,” as use herein, generally refers to a polynucleotide of interest under study. In certain embodiments, a target polynucleotide contains one or more sequences that are of interest and under study. A target polynucleotide can comprise, for example, a genomic sequence. The target polynucleotide can comprise a target sequence whose presence, amount, and/or nucleotide sequence, or changes in these, are desired to be determined.

The target polynucleotide can be a region of gene associated with a disease. In some embodiments, the region is an exon. In some embodiments, the gene is a druggable target. The term “druggable target”, as used herein, generally refers to a gene or cellular pathway that is modulated by a disease therapy. The disease can be cancer. Accordingly, the gene can be a known cancer-related gene. In some embodiments, the cancer-related gene is selected from the group consisting of ABCA1, BRAF, CHD5, EP300, FLT1, ITPA, MYC, P1K3R1, SKP2, TP53, ABCA7, BRCA1, CHEK1, EPHA3, FLT3, JAK1, MYCL1, PIK3R2, SLC19A1, TP73, ABCB1, BRCA2, CHEK2, EPHA5, FLT4, JAK2, MYCN, PKHD1, SLC1A6, TPM3, ABCC2, BRIP1, CLTC, EPHA6, FN1, JAK3, MYH2, PLCB1, SLC22A2, TPMT, ABCC3, BUB1B, COL1A1, EPHA7, FOS, JUN, MYH9, PLCG1, SLCO1B3, TPO, ABCC4, Clorf144, COPS5, EPHA8, FOXO1, KBTBD11, NAV3, PLCG2, SMAD2, TPR, ABCG2, CABLES1, CREB1, EPHB1, FOXO3, KDM6A, NBN, PML, SMAD3, TR10, ABL1, CACNA2D1, CREBBP, EPHB4, FOXP4, KDR, NCOA2, PMS2, SMAD4, TRRAP, ABL2, CAMKV, CRKL, EPHB6, GAB1, KIT, NEK11, PPARG, SMARCA4, TSC1, ACVR1B, CARD11, CRLF2, EPO, GATA1, KLF6, NF1, PPARGC1A, SMARCB1, TSC2, ACVR2A, CARM1, CSF1R, ERBB2, GLI1, KLHDC4, NF2, PPP1R3A, SMO, TTK, ADCY9, CAV1, CSMD3, ERBB3, GLI3, KRAS, NKX2-1, PPP2R1A, SOCS1, TYK2, AGAP2, CBFA2T3, CSNK1G2, ERBB4, GNA11, LMO2, NOS2, PPP2R1B, SOD2, TYMS, AKT1, CBL, CTNNA1, ERCC1, GNAQ, LRP1B, NOS3, PRKAA2, SOS1, UGT1A1, AKT2, CCND1, CTNNA2, ERCC2, GNAS, LRP2, NOTCH1, PRKCA, SOX10, UMPS, AKT3, CCND2, CTNNB1, ERCC3, GPR124, LRP6, NOTCH2, PRKCZ, SOX2, USP9X, ALK, CCND3, CYFIP1, ERCC4, GPR133, LTK, NOTCH3, PRKDC, SP1, VEGF, ANAPC5, CCNE1, CYLD, ERCC5, GRB2, MAN1B1, NPM1, PTCH1, SPRY2, VEGFA, APC, CD40LG, CYP19A1, ERCC6, GSK3B, MAP2K1, NQO1, PTCH2, SRC, VHL, APC2, CD44, CYP1B1, ERG, GSTP1, MAP2K2, NR3Cl, PTEN, ST6GAL2, WRN, AR, CD79A, CYP2C19, ERN2, GUCY1A2, MAP2K4, NRAS, PTGS2, STAT1, WT1, ARAF, CD79B, CYP2C8, ESR1, HDAC1, MAP2K7, NRP2, PTPN11, STAT3, XPA, ARFRP1, CDC42, CYP2D6, ESR2, HDAC2, MAP3K1, NTRK1, PTPRB, STK11, XPC, ARID1A, CDC42BPB, CYP3A4, ETV4, HGF, MAPK1, NTRK2, PTPRD, SUFU, ZFY, ATM, CDC73, CYP3A5, EWSR1, HIF1A, MAPK3, NTRK3, RAD50, SULT1A1, ZNF521, ATP5A1, CDH1, DACH2, EXT1, HM13, MAPK8, OMA1, RAD51, SUZ12, ATR, CDH10, DCC, EZH2, HMGA1, MARK3, OR10R2, RAFT, TAF1, AURKA, CDH2, DCLK3, FANCA, HNF1A, MCL1, PAK3, RARA, TBX22, AURKB, CDH2O, DDB2, FANCD2, HOXA3, MDM2, PARP1, RB1, TCF12, BAI3, CDH5, DDR2, FANCE, HOXA9, MDM4, PAX5, REM1, TCF3, BAP1, CDK2, DGKB, FANCF, HRAS, MECOM, PCDH15, RET, TCF4, BARD1, CDK4, DGKZ, FAS, HSP90AA1, MEN1, PCDH18, RICTOR, TEK, BAX, CDK6, DIRAS3, FBXW7, IDH1, MET, PCNA, RIPK1, TEP1, BCL11A, CDK7, DLG3, FCGR3A, IDH2, MITF, PDGFA, ROR1, TERT, BCL2, CDK8, DLL1, FES, IFNG, MLH1, PDGFB, ROR2, TET2, BCL2A1, CDKN1A, DNMT1, FGFR1, IGF1R, MLL, PDGFRA, ROS1, TGFBR2, BCL2L1, CDKN1B, DNMT3A, FGFR2, IGF2R, MLL3, PDGFRB, RPS6KA2, THBS1, BCL2L2, CDKN2A, DNMT3B, FGFR3, IKBKE, MPL, PDZRN3, RPTOR, TNFAIP3, BCL3, CDKN2B, DOT1L, FGFR4, IKZF1, MRE11A, PHLPP2, RSPO2, TNKS, BCL6, CDKN2C, DPYD, FH, IL2RG, MSH2, PIK3C3, RSPO3, TNKS2, BCR, CDKN2D, E2F1, FHOD3, INHBA, MSH6, PIK3CA, RUNX1, TNNI3K, BIRC5, CDX2, EED, FIGF, INSR, MTHFR, PIK3CB, SDHB, TNR, BIRC6, CEBPA, EGF, FLG2, IRS1, MTOR, PIK3CD, SF3B1, TOP1, BLM, CERK, EGFR, FLNC, IRS2, MUTYH, PIK3CG, SHC1, and TOP2A.

The term “genomic sequence”, as used herein, generally refers to a sequence that occurs in a genome. Because RNAs are transcribed from a genome, this term encompasses sequence that exist in the nuclear genome of an organism, as well as sequences that are present in a cDNA copy of an RNA (e.g., an mRNA) transcribed from such a genome.

The terms “anneal”, “hybridize” or “bind,” can refer to two polynucleotide sequences, segments or strands, and can be used interchangeably and have the usual meaning in the art. Two complementary sequences (e.g., DNA and/or RNA) can anneal or hybridize by forming hydrogen bonds with complementary bases to produce a double-stranded polynucleotide or a double-stranded region of a polynucleotide.

As used herein, the term “complementary” generally refers to a relationship between two antiparallel nucleic acid sequences in which the sequences are related by the base-pairing rules: A pairs with T or U and C pairs with G. A first sequence or segment that is “perfectly complementary” to a second sequence or segment is complementary across its entire length and has no mismatches. A first sequence or segment is “substantially complementary” to a second sequence of segment when a polynucleotide consisting of the first sequence is sufficiently complementary to specifically hybridize to a polynucleotide consisting of the second sequence.

The term “duplex,” or “duplexed,” as used herein, can describe two complementary polynucleotides that are base-paired, i.e., hybridized together.

As used herein, “amplification” of a nucleic acid sequence generally refers to in vitro techniques for enzymatically increasing the number of copies of a target sequence. Amplification methods include both asymmetric methods (in which the predominant product is single-stranded) and conventional methods (in which the predominant product is double-stranded). A “round” or “cycle” of amplification generally refers to a PCR cycle in which a double stranded template DNA molecule is denatured into single-stranded templates, forward and reverse primers are hybridized to the single stranded templates to form primer/template duplexes, primers are extended by a DNA polymerase from the primer/template duplexes to form extension products. In subsequent rounds of amplification the extension products are denatured into single stranded templates and the cycle is repeated.

The terms “template”, “template strand”, “template DNA” and “template nucleic acid” can be used interchangeably herein to refer to a strand of DNA that is copied by an amplification cycle.

The term “denaturing,” as used herein, generally refers to the separation of a nucleic acid duplex into two single strands.

The term “extending”, as used herein, generally refers to the extension of a primer hybridized to a template nucleic acid by the addition of nucleotides using an enzyme, e.g., a polymerase.

A “primer” is generally a nucleotide sequence (e.g., an oligonucleotide), generally with a free 3′-OH group, that hybridizes with a template sequence (such as a target polynucleotide, or a primer extension product) and is capable of promoting polymerization of a polynucleotide complementary to the template. A primer can be, for example, a sequence of the template (such as a primer extension product or a fragment of the template created following RNase cleavage of a template-DNA complex) that is hybridized to a sequence in the template itself (for example, as a hairpin loop), and that is capable of promoting nucleotide polymerization. Thus, a primer can be an exogenous (e.g., added) primer or an endogenous (e.g., template fragment) primer.

The terms “determining”, “measuring”, “evaluating”, “assessing,” “assaying,” and “analyzing” can be used interchangeably herein to refer to any form of measurement, and include determining if an element is present or not. These terms can include both quantitative and/or qualitative determinations. Assessing may be relative or absolute. “Assessing the presence of” can include determining the amount of something present, as well as determining whether it is present or absent.

As used herein, the term “Tm” generally refers to the melting temperature of an oligonucleotide duplex at which half of the duplexes remain hybridized and half of the duplexes dissociate into single strands. See Sambrook and Russell (2001; Molecular Cloning: A Laboratory Manual, 3.sup.rd ed., Cold Spring Harbor Press, Cold Spring Harbor N.Y., ch. 10).

The term “free in solution,” as used here, can describe a molecule, such as a polynucleotide, that is not bound or tethered to a solid support.

The term “genomic fragment”, as used herein, can refer to a region of a genome, e.g., an animal or plant genome such as the genome of a human, monkey, rat, fish or insect or plant. A genomic fragment may or may not be adaptor ligated. A genomic fragment may be adaptor ligated (in which case it has an adaptor ligated to one or both ends of the fragment, to at least the 5′ end of a molecule), or non-adaptor ligated.

The term “ligating”, as used herein, can refer to the enzymatically catalyzed joining of the terminal nucleotide at the 5′ end of a first DNA molecule to the terminal nucleotide at the 3′ end of a second DNA molecule.

A “primer binding site” can refer to a site to which a primer hybridizes in an oligonucleotide or a complementary strand thereof.

The term “separating”, as used herein, can refer to physical separation of two elements (e.g., by size, affinity, degradation of one element etc.).

The term “sequencing”, as used herein, can refer to a method by which the identity of at least 10 consecutive nucleotides (e.g., the identity of at least 20, at least 50, at least 100, at least 200, or at least 500 or more consecutive nucleotides) of a polynucleotide are obtained.

The term “adaptor-ligated”, as used herein, can refer to a nucleic acid that has been ligated to an adaptor. The adaptor can be ligated to a 5′ end or a 3′ end of a nucleic acid molecule, or can be added to an internal region of a nucleic acid molecule.

The term “bridge PCR” can refer to a solid-phase polymerase chain reaction in which the primers that are extended in the reaction are tethered to a substrate by their 5′ ends. During amplification, the amplicons form a bridge between the tethered primers. Bridge PCR (which may also be referred to as “cluster PCR”) is used in Illumina's Solexa platform. Bridge PCR and Illumina's Solexa platform are generally described in a variety of publications, e.g., Gudmundsson et al (Nat. Genet. 2009 41:1122-6), Out et al (Hum. Mutat. 2009 30:1703-12) and Turner (Nat. Methods 2009 6:315-6), U.S. Pat. No. 7,115,400, and publication application publication nos. US20080160580 and US20080286795.

The term “barcode sequence” as used herein, generally refers to a unique sequence of nucleotides that can encode information about an assay. A barcode sequence can encode information relating to the identity of an interrogated allele, identity of a target polynucleotide or genomic locus, identity of a sample, a subject, or any combination thereof. A barcode sequence can be a portion of a primer, a reporter probe, or both. A barcode sequence may be at the 5′-end or 3′-end of an oligonucleotide, or may be located in any region of the oligonucleotide. A barcode sequence generally is not part of a template sequence. Barcode sequences may vary widely in size and composition; the following references provide guidance for selecting sets of barcode sequences appropriate for particular embodiments: Brenner, U.S. Pat. No. 5,635,400; Brenner et al, Proc. Natl. Acad. Sci., 97: 1665-1670 (2000); Shoemaker et al, Nature Genetics, 14: 450-456 (1996); Morris et al, European patent publication 0799897A1; Wallace, U.S. Pat. No. 5,981,179. A barcode sequence may have a length of about 4 to 36 nucleotides, about 6 to 30 nucleotides, or about 8 to 20 nucleotides.

The term “mutation”, as used herein, generally refers to a change of the nucleotide sequence of a genome. Mutations can involve large sections of DNA (e.g., copy number variation). Mutations can involve whole chromosomes (e.g., aneuploidy). Mutations can involve small sections of DNA. Examples of mutations involving small sections of DNA include, e.g., point mutations or single nucleotide polymorphisms, multiple nucleotide polymorphisms, insertions (e.g., insertion of one or more nucleotides at a locus), multiple nucleotide changes, deletions (e.g., deletion of one or more nucleotides at a locus), and inversions (e.g., reversal of a sequence of one or more nucleotides).

The term “locus”, as used herein, can refer to a location of a gene, nucleotide, or sequence on a chromosome. An “allele” of a locus, as used herein, can refer to an alternative form of a nucleotide or sequence at the locus. A “wild-type allele” generally refers to an allele that has the highest frequency in a population of subjects. A “wild-type” allele generally is not associated with a disease. A “mutant allele” generally refers to an allele that has a lower frequency that a “wild-type allele” and may be associated with a disease. A “mutant allele” may not have to be associated with a disease. The term “interrogated allele” generally refers to the allele that an assay is designed to detect.

The term “single nucleotide polymorphism”, or “SNP”, as used herein, generally refers to a type of genomic sequence variation resulting from a single nucleotide substitution within a sequence. “SNP alleles” or “alleles of a SNP” generally refer to alternative forms of the SNP at particular locus. The term “interrogated SNP allele” generally refers to the SNP allele that an assay is designed to detect.

In certain cases, an oligonucleotide used in the method described herein may be designed using a reference genomic region, i.e., a genomic region of known nucleotide sequence, e.g., a chromosomal region whose sequence is deposited at NCBI's Genbank database or other database, for example.

A “plurality” generally contains at least 2 members. In certain cases, a plurality may have at least 10, at least 100, at least 100, at least 10,000, at least 100,000, at least 1000000, at least 10000000, at least 100000000, or at least 1000000000 or more members.

The term “separating”, as used herein, generally refers to physical separation of two elements (e.g., by cleavage, hydrolysis, or degradation of one of the two elements).

The terms “label” and “detectable moiety” can be used interchangeably herein to refer to any atom or molecule which can be used to provide a detectable signal, and which can be attached to a nucleic acid or protein. Labels may provide signals detectable by fluorescence, radioactivity, colorimetry, gravimetry, X-ray diffraction or absorption, magnetism, enzymatic activity, and the like.

### Overview

Aspects of the invention relate to methods and kits that improve the monitoring and treatment of a subject suffering from a disease. The disease can be a cancer, e.g., a tumor, a leukemia such as acute leukemia, acute t-cell leukemia, acute lymphocytic leukemia, acute myelocytic leukemia, myeloblastic leukemia, promyelocytic leukemia, myelomonocytic leukemia, monocytic leukemia, erythroleukemia, chronic leukemia, chronic myelocytic (granulocytic) leukemia, or chronic lymphocytic leukemia, polycythemia vera, lymphomas such as Hodgkin's lymphoma, follicular lymphoma or non-Hodgkin's lymphoma, multiple myeloma, Waldenstrom's macroglobulinemia, heavy chain disease, solid tumors, sarcomas, carcinomas such as, e.g., fibrosarcoma, myxosarcoma, liposarcoma, chondrosarcoma, osteogenic sarcoma, lymphangiosarcoma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic, carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, craniopharyngioma, ependymoma, pinealoma, hemangioblastoma, acoustic neuroma, oligodendroglioma, meningioma, melanoma, neuroblastoma, retinoblastoma, endometrial cancer, non small cell lung cancer,

The subject can be suspected or known to harbor a solid tumor, or can be a subject who previously harbored a solid tumor.

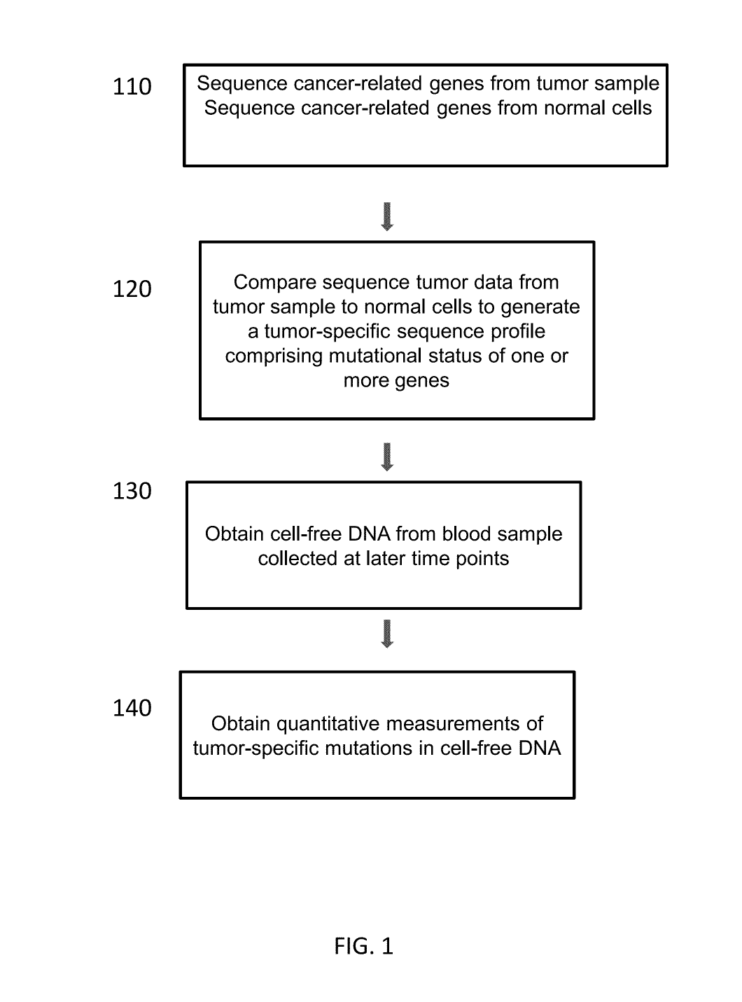

FIG. 1 depicts an exemplary workflow of a method for assessing cancer. In step 110, the method comprises sequencing cancer-related genes from a tumor sample isolated from said subject and sequencing a set of cancer-related genes from normal cells isolated from said subject. The tumor sample can be a solid tumor sample. The normal cells can be blood cells isolated from a blood sample from the subject. In step 120, sequence data from the tumor can be compared to sequence data from normal cells to generate a tumor-specific sequence profile. In some embodiments, the tumor-specific sequence profile comprises mutational status of one or more genes in the set. The method can further comprise generating a report describing the tumor-specific sequence profile. In some embodiments, the method further comprises choosing a subset of 2-4 genes known to harbor tumor-specific mutations for further monitoring. In step 130, cell-free DNA is obtained from a blood sample collected from the subject prior to treatment (e.g., tumor removal or therapeutic intervention) as well as prior to treatment (tumor removal or therapeutic intervention) as well as at a later time point. In step 140, the cell-free DNA from the blood sample is assayed for the 2-4 genes in the subset to obtain quantitative measurement of the tumor-specific mutations.

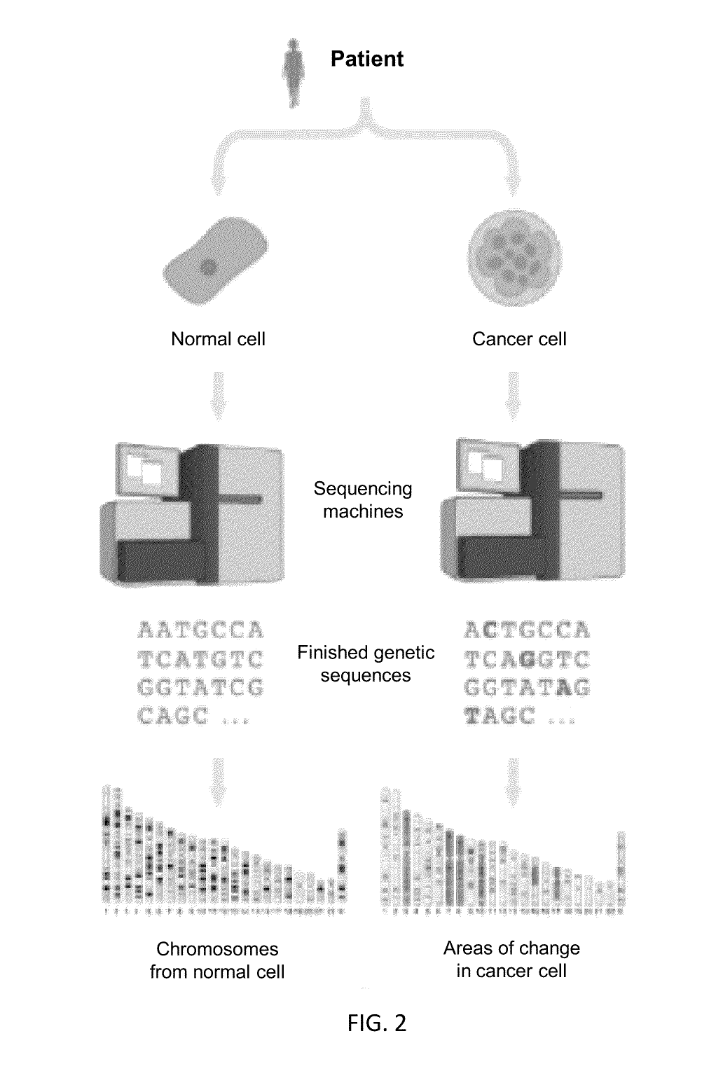

FIG. 2 is a depiction of an exemplary workflow of a method as described in FIG. 1, from steps 110-120, for sequencing a tumor cell and a normal cell in a subject.

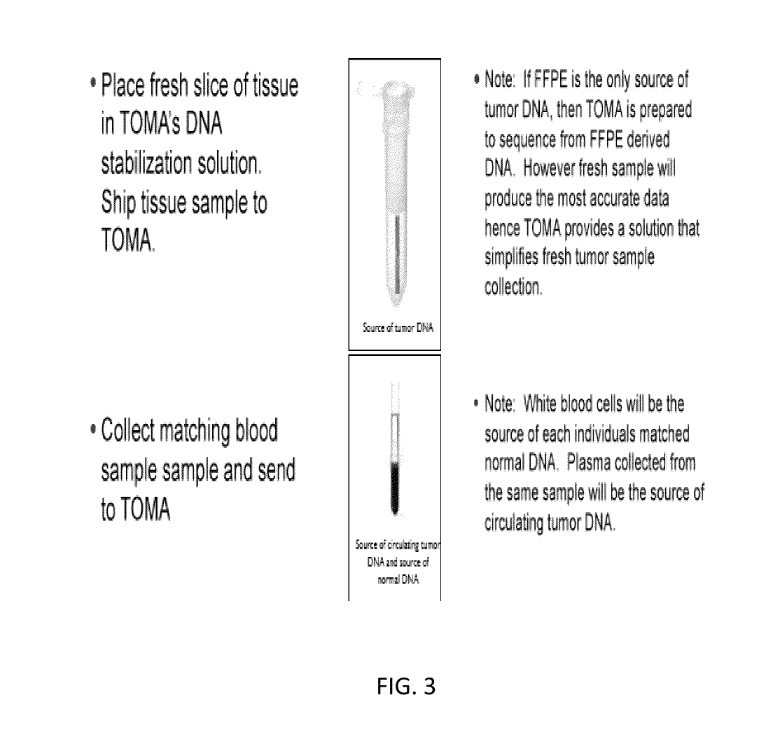

The tumor sample is processed prior to sequencing by fixation in a formalin solution, followed by embedding in paraffin (e.g., is a FFPE sample). In other embodiments, the tumor sample is frozen prior to sequencing. In yet other embodiments, the tumor sample is neither fixed nor frozen. The unfixed, unfrozen tumor sample is stored in a storage solution configured for the preservation of nucleic acid at room temperature, as depicted in FIG. 3. The storage solution can be a commercially available storage solution. Exemplary storage solutions include, but are not limited to, DNA storage solutions from Biomatrica (see, e.g., WO/2012/018638, WO/2009/038853, US20080176209).

Further embodiments of the sequencing methods and assays for determining mutational status in the blood are described herein.

### Next-Generation Sequencing

In some embodiments, the tumor sample and normal cells from the subject are sequenced. In some embodiments, nucleic acid is isolated from the tumor sample and normal cells using any methods known in the art. The nucleic acid is DNA. The DNA from the tumor sample and normal cells can be used to prepare a subject-specific tumor DNA library and/or normal DNA library. DNA libraries can be used for sequencing by a sequencing platform. The sequencing platform can be a next-generation sequencing (NGS) platform. In some embodiments, the method further comprises sequencing the nucleic acid libraries using NGS technology. NGS technology can involve sequencing of clonally amplified DNA templates or single DNA molecules in a massively parallel fashion (e.g. as described in Volkerding et al. Clin Chem 55:641-658 [2009]; Metzker M Nature Rev 11:31-46 [2010]). In addition to high-throughput sequence information, NGS provides digital quantitative information, in that each sequence read is a countable “sequence tag” representing an individual clonal DNA template or a single DNA molecule.

### Next Generation Sequencing Platforms

The next-generation sequencing platform can be a commercially available platform. Commercially available platforms include, e.g., platforms for sequencing-by-synthesis, ion semiconductor sequencing, pyrosequencing, reversible dye terminator sequencing, sequencing by ligation, single-molecule sequencing, sequencing by hybridization, and nanopore sequencing. Platforms for sequencing by synthesis are available from, e.g., Illumina, 454 Life Sciences, Helicos Biosciences, and Qiagen. Illumina platforms can include, e.g., Illumina's Solexa platform, Illumina's Genome Analyzer, and are described in Gudmundsson et al (Nat. Genet. 2009 41:1122-6), Out et al (Hum. Mutat. 2009 30:1703-12) and Turner (Nat. Methods 2009 6:315-6), U.S. Patent Application Pub nos. US20080160580 and US20080286795, U.S. Pat. Nos. 6,306,597, 7,115,400, and 7232656. 454 Life Science platforms include, e.g., the GS Flex and GS Junior, and are described in U.S. Pat. No. 7,323,305. Platforms from Helicos Biosciences include the True Single Molecule Sequencing platform. Platforms for ion seminconductor sequencing include, e.g., the Ion Torrent Personal Genome Machine (PGM) and are described in U.S. Pat. No. 7,948,015. Platforms for pryosequencing include the GS Flex 454 system and are described in U.S. Pat. Nos. 7,211,390; 7,244,559; 7264929. Platforms and methods for sequencing by ligation include, e.g., the SOLiD sequencing platform and are described in U.S. Pat. No. 5,750,341. Platforms for single-molecule sequencing include the SMRT system from Pacific Bioscience and the Helicos True Single Molecule Sequencing platform.

While the automated Sanger method is considered as a ‘first generation’ technology, Sanger sequencing including the automated Sanger sequencing, can also be employed by the method of the invention. Additional sequencing methods that comprise the use of developing nucleic acid imaging technologies e.g. atomic force microscopy (AFM) or transmission electron microscopy (TEM), are also encompassed by the method of the invention. Exemplary sequencing technologies are described below.

The DNA sequencing technology can utilize the Ion Torrent sequencing platform, which pairs semiconductor technology with a sequencing chemistry to directly translate chemically encoded information (A, C, G, T) into digital information (0, 1) on a semiconductor chip. Without wishing to be bound by theory, when a nucleotide is incorporated into a strand of DNA by a polymerase, a hydrogen ion is released as a byproduct. The Ion Torrent platform detects the release of the hydrogen atom as a change in pH. A detected change in pH can be used to indicate nucleotide incorporation. The Ion Torrent platform comprises a high-density array of micro-machined wells to perform this biochemical process in a massively parallel way. Each well holds a different library member, which may be clonally amplified. Beneath the wells is an ion-sensitive layer and beneath that an ion sensor. The platform sequentially floods the array with one nucleotide after another. When a nucleotide, for example a C, is added to a DNA template and is then incorporated into a strand of DNA, a hydrogen ion will be released. The charge from that ion will change the pH of the solution, which can be identified by Ion Torrent's ion sensor. If the nucleotide is not incorporated, no voltage change will be recorded and no base will be called. If there are two identical bases on the DNA strand, the voltage will be double, and the chip will record two identical bases called. Direct identification allows recordation of nucleotide incorporation in seconds. Library preparation for the Ion Torrent platform generally involves ligation of two distinct adaptors at both ends of a DNA fragment.

The DNA sequencing technology utilizes an Illumina sequencing platform, which generally employs cluster amplification of library members onto a flow cell and a sequencing-by-synthesis approach. Cluster-amplified library members are subjected to repeated cycles of polymerase-directed single base extension. Single-base extension can involve incorporation of reversible-terminator dNTPs, each dNTP labeled with a different removable fluorophore. The reversible-terminator dNTPs are generally 3′ modified to prevent further extension by the polymerase. After incorporation, the incorporated nucleotide can be identified by fluorescence imaging. Following fluorescence imaging, the fluorophore can be removed and the 3′ modification can be removed resulting in a a 3′ hydroxyl group, thereby allowing another cycle of single base extension. Library preparation for the Illumina platform generally involves ligation of two distinct adaptors at both ends of a DNA fragment.

The DNA sequencing technology that is used in the method of the invention can be the Helicos True Single Molecule Sequencing (tSMS), which can employ sequencing-by-synthesis technology. In the tSMS technique, a polyA adaptor can be ligated to the 3′end of DNA fragments. The adapted fragments can be hybridized to poly-T oligonucleotides immobilized on the tSMS flow cell. The library members can be immobilized onto the flow cell at a density of about 100 million templates/cm2. The flow cell can be then loaded into an instrument, e.g., HeliScope™ sequencer, and a laser can illuminate the surface of the flow cell, revealing the position of each template. A CCD camera can map the position of the templates on the flow cell surface. The library members can be subjected to repeated cycles of polymerase-directed single base extension. The sequencing reaction begins by introducing a DNA polymerase and a fluorescently labeled nucleotide. The polymerase can incorporate the labeled nucleotides to the primer in a template directed manner. The polymerase and unincorporated nucleotides can be removed. The templates that have directed incorporation of the fluorescently labeled nucleotide can be discerned by imaging the flow cell surface. After imaging, a cleavage step can remove the fluorescent label, and the process can be repeated with other fluorescently labeled nucleotides until a desired read length is achieved. Sequence information can be collected with each nucleotide addition step.

The DNA sequencing technology can utilize a 454 sequencing platform (Roche) (e.g. as described in Margulies, M. et al. Nature 437:376-380 [2005]). 454 sequencing generally involves two steps. In a first step, DNA can be sheared into fragments. The fragments can be blunt-ended. Oligonucleotide adaptors can be ligated to the ends of the fragments. The adaptors generally serve as primers for amplification and sequencing of the fragments. At least one adaptor can comprise a capture reagent, e.g., a biotin. The fragments can be attached to DNA capture beads, e.g., streptavidin-coated beads. The fragments attached to the beads can be PCR amplified within droplets of an oil-water emulsion, resulting in multiple copies of clonally amplified DNA fragments on each bead. In a second step, the beads can be captured in wells, which can be pico-liter sized. Pyrosequencing can be performed on each DNA fragment in parallel. Pyrosequencing generally detects release of pyrophosphate (PPi) upon nucleotide incorporation. PPi can be converted to ATP by ATP sulfurylase in the presence of adenosine 5′ phosphosulfate. Luciferase can use ATP to convert luciferin to oxyluciferin, thereby generating a light signal that is detected. A detected light signal can be used to identify the incorporated nucleotide.

The DNA sequencing technology can utilize a SOLiD™ technology (Applied Biosystems). The SOLiD platform generally utilizes a sequencing-by-ligation approach. Library preparation for use with a SOLiD platform generally comprises ligation of adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations can be prepared in microreactors containing beads, primers, template, and PCR components. Following PCR, the templates can be denatured. Beads can be enriched for beads with extended templates. Templates on the selected beads can be subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide can be removed and the process can then be repeated.

The DNA sequencing technology can utilize a single molecule, real-time (SMRT™) sequencing platform (Pacific Biosciences). In SMRT sequencing, the continuous incorporation of dye-labeled nucleotides can be imaged during DNA synthesis. Single DNA polymerase molecules can be attached to the bottom surface of individual zero-mode wavelength identifiers (ZMW identifiers) that obtain sequence information while phospholinked nucleotides are being incorporated into the growing primer strand. A ZMW generally refers to a confinement structure which enables observation of incorporation of a single nucleotide by DNA polymerase against a background of fluorescent nucleotides that rapidly diffuse in an out of the ZMW on a microsecond scale. By contrast, incorporation of a nucleotide generally occurs on a milliseconds timescale. During this time, the fluorescent label can be excited to produce a fluorescent signal, which is detected. Detection of the fluorescent signal can be used to generate sequence information. The fluorophore can then be removed, and the process repeated. Library preparation for the SMRT platform generally involves ligation of hairpin adaptors to the ends of DNA fragments.

The DNA sequencing technology can utilize nanopore sequencing (e.g. as described in Soni GV and Meller A. Clin Chem 53: 1996-2001 [2007]). Nanopore sequencing DNA analysis techniques are being industrially developed by a number of companies, including Oxford Nanopore Technologies (Oxford, United Kingdom). Nanopore sequencing is a single-molecule sequencing technology whereby a single molecule of DNA is sequenced directly as it passes through a nanopore. A nanopore can be a small hole, of the order of 1 nanometer in diameter. Immersion of a nanopore in a conducting fluid and application of a potential (voltage) across can result in a slight electrical current due to conduction of ions through the nanopore. The amount of current which flows is sensitive to the size and shape of the nanopore and to occlusion by, e.g., a DNA molecule. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore represents a reading of the DNA sequence.

The DNA sequencing technology can utilize a chemical-sensitive field effect transistor (chemFET) array (e.g., as described in U.S. Patent Application Publication No. 20090026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be discerned by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

The DNA sequencing technology can utilize transmission electron microscopy (TEM). The method, termed Individual Molecule Placement Rapid Nano Transfer (IMPRNT), generally comprises single atom resolution transmission electron microscope imaging of high-molecular weight (150 kb or greater) DNA selectively labeled with heavy atom markers and arranging these molecules on ultra-thin films in ultra-dense (3 nm strand-to-strand) parallel arrays with consistent base-to-base spacing. The electron microscope is used to image the molecules on the films to determine the position of the heavy atom markers and to extract base sequence information from the DNA. The method is further described in PCT patent publication WO 2009/046445. The method allows for sequencing complete human genomes in less than ten minutes.

The method can utilize sequencing by hybridization (SBH). SBH generally comprises contacting a plurality of polynucleotide sequences with a plurality of polynucleotide probes, wherein each of the plurality of polynucleotide probes can be optionally tethered to a substrate. The substrate might be flat surface comprising an array of known nucleotide sequences. The pattern of hybridization to the array can be used to determine the polynucleotide sequences present in the sample. In other embodiments, each probe is tethered to a bead, e.g., a magnetic bead or the like. Hybridization to the beads can be identified and used to identify the plurality of polynucleotide sequences within the sample.

The length of the sequence read can vary depending on the particular sequencing technology utilized. NGS platforms can provide sequence reads that vary in size from tens to hundreds, or thousands of base pairs. In some embodiments of the method described herein, the sequence reads are about 20 bases long, about 25 bases long, about 30 bases long, about 35 bases long, about 40 bases long, about 45 bases long, about 50 bases long, about 55 bases long, about 60 bases long, about 65 bases long, about 70 bases long, about 75 bases long, about 80 bases long, about 85 bases long, about 90 bases long, about 95 bases long, about 100 bases long, about 110 bases long, about 120 bases long, about 130, about 140 bases long, about 150 bases long, about 200 bases long, about 250 bases long, about 300 bases long, about 350 bases long, about 400 bases long, about 450 bases long, about 500 bases long, about 600 bases long, about 700 bases long, about 800 bases long, about 900 bases long, about 1000 bases long, or more than 1000 bases long.

Partial sequencing of DNA fragments present in the sample can be performed, and sequence tags comprising reads that map to a known reference genome can be counted. Only sequence reads that uniquely align to the reference genome can be counted as sequence tags. In one embodiment, the reference genome is the human reference genome NCBI36/hgl 8 sequence, which is available on the world wide web at genome.ucsc.edu/cgi-bin/hgGateway?org=Human&db=hgl 8&hgsid=166260105). Other sources of public sequence information include GenBank, dbEST, dbSTS, EMBL (the European Molecular Biology Laboratory), and the DDBJ (the DNA Databank of Japan). The reference genome can also comprise the human reference genome NCBI36/hgl 8 sequence and an artificial target sequences genome, which includes polymorphic target sequences e.g. a SNP genome comprising SEQ ID NOs: 1-56. In yet another embodiment, the reference genome is an artificial target sequence genome comprising polymorphic target sequences.

Mapping of the sequence tags can be achieved by comparing the sequence of the tag with the sequence of the reference genome to determine the chromosomal origin of the sequenced nucleic acid (e.g. cell free DNA) molecule, and specific genetic sequence information is not needed. A number of computer algorithms are available for aligning sequences, including without limitation BLAST (Altschul et al., 1990), BLITZ (MPsrch) (Sturrock & Collins, 1993), FASTA (Person & Lipman, 1988), BOWTIE (Langmead et al, Genome Biology 10:R25.1-R25.10 [2009]), or ELAND (Illumina, Inc., San Diego, Calif., USA). In one embodiment, one end of the clonally expanded copies of the DNA molecule is sequenced and processed by bioinformatic alignment analysis for the Illumina Genome Analyzer, which uses the Efficient Large-Scale Alignment of Nucleotide Databases (ELAND) software. Additional software includes SAMtools (SAMtools, Bioinformatics, 2009, 25(16):2078-9), and the Burroughs-Wheeler block sorting compression procedure which involves block sorting or preprocessing to make compression more efficient.

The sequencing platforms described herein generally comprise a solid support immobilized thereon surface-bound oligonucleotides which allow for the capture and immobilization of sequencing library members to the solid support. Surface bound oligonucleotides generally comprise sequences complementary to the adaptor sequences of the sequencing library.

The DNA samples can be used to prepare nucleic acid libraries for sequencing. Preparation of nucleic acid libraries can comprise any method known in the art or as described herein. As used herein, the terms “library” or “sequencing library” are used interchangeably herein and can refer to a plurality of nucleic acid fragments obtained from a biological sample. Generally, the fragments are modified with an adaptor sequence which affects coupling (e.g., capture and/or immobilization) of the fragments to a sequencing platform. An adaptor sequence can comprise a defined oligonucleotide sequence that affects coupling of a library member to a sequencing platform. By way of example only, the adaptor can comprise a sequence that is at least 25% complementary or identical to an oligonucleotide sequence immobilized onto a solid support (e.g., a sequencing flow cell or bead). An adaptor sequence can comprise a defined oligonucleotide sequence that is at least 70% complementary or identical to a sequencing primer. The sequencing primer can enable nucleotide incorporation by a polymerase, wherein incorporation of the nucleotide is monitored to provide sequencing information. The sequencing primer can be about 15-25 bases. In some embodiments, the sequencing primer is conjugated to the 3′ end of the adaptor. In some embodiments, an adaptor comprises a sequence that is at least 25% complementary or identical to an oligonucleotide sequence immobilized onto a solid support and a sequence that is at least 70% complementary or identical to a sequencing primer. Coupling can also be achieved through serially stitching adaptors together. The number of adaptors that can be stitched can be 1, 2, 3, 4 or more. The stitched adaptors can be at least 35 bases, 70 bases, 105 bases, 140 bases or more.

The adaptor can comprise a barcode sequence. At least 50%, 60%, 70%, 80%, 90%, or 100% of sequencing library members in a library can comprise the same adaptor sequence. At least 50%, 60%, 70%, 80%, 90%, or 100% of the ssDNA library members can comprise an adaptor sequence at a first end but not at a second end. In some embodiments, the first end is a 5′ end. In some embodiments, the first end is at 3′ end. The adaptor sequence can be chosen by a user according to the sequencing platform used for sequencing. By way of example only, an Illumina sequencing by synthesis platform comprises a solid support with a first and second population of surface-bound oligonucleotides immobilized thereon. Such oligonucleotides comprise a sequence for hybridizing to a first and second Illumina-specific adaptor oligonucleotide and priming an extension reaction. Accordingly, a DNA library member can comprise a first Illumina-specific adaptor that is partially or wholly complementary to a first population of surface bound oligonucleotides of an Illumina system. By way of other example only, the SOLiD system, and Ion Torrent, GS FLEX system comprises a solid support in the form of a bead with a single population of surface bound oligonucleotides immobilized thereon. Accordingly, in some embodiments the ssDNA library member comprises an adaptor sequence that is complementary to a surface-bound oligonucleotide of a SOLiD system, Ion Torrent system, or GS Flex system.

Accordingly, in one aspect, the invention provides improved methods of preparing a nucleic acid library. The nucleic acid library can be a DNA library. The method can comprise ligation of adaptor sequences to DNA fragments. The method can improve efficiency of adaptor ligation by at least 10-fold. In some embodiments, the nucleic acid library is a ssDNA library. In some embodiments, the nucleic acid library is a partial ssDNA library.

ssDNA Fragment/ssDNA Library Preparation

In some embodiments, the ssDNA fragment is a member of a ssDNA library. The single-stranded nucleic acid library is prepared from a sample of double-stranded nucleic acid using any means known in the art or described herein.

The starting sample can be a biological sample obtained from a subject. Exemplary subjects and biological samples are described herein.

The nucleic acid sample can be enriched for target polynucleotides. Target enrichment can be by any means known in the art. For example, the nucleic acid sample may be enriched by amplifying target sequences using target-specific primers. The target amplification can occur in a digital PCR format, using any methods or systems known in the art. The nucleic acid sample may be enriched by capture of target sequences onto an array immobilized thereon target-selective oligonucleotides. The nucleic acid sample may be enriched by hybridizing to target-selective oligonucleotides free in solution. The oligonucleotides may comprise a capture moiety which enables capture by a capture reagent. Exemplary capture moieties and capture reagents are described herein. In some embodiments, the nucleic acid sample is not enriched for target polynucleotides, e.g., represents a whole genome.

Accordingly, in some aspects the invention provides a method of preparing a ssDNA library. The method can comprise denaturing a double stranded DNA fragment into ssDNA fragments, ligating a primer docking sequence onto one end of the ssDNA fragment, hybridizing a primer to the primer docking sequence. The primer can comprise at least a portion of an adaptor sequence that couples to a next-generation sequencing platform. The method can further comprise extension of the hybridized primer to create a duplex, wherein the duplex comprises the original ssDNA fragment and an extended primer strand. The extended primer strand can be separated from the original ssDNA fragment. The extended primer strand can be collected, wherein the extended primer strand is a member of the ssDNA library.

dsDNA can be fragmented by any means known in the art or as described herein. dsDNA can be fragmented, for example, by mechanical shearing, by nebulization, or by sonication.

In some embodiments, cDNA is generated from RNA using random primed reverse transcription (RNaseH+) to generate randomly sized cDNA.

The dsDNA fragments or randomly sized cDNA can be less than 1000 bp, less than 800 bp, less than 700 bp, less than 600 bp, less than 500 bp, less than 400 bp, less than 300 bp, less than 200 bp, or less than 100 bp. The DNA fragments can be about 40-100 bp, about 50-125 bp, about 100-200 bp, about 150-400 bp, about 300-500 bp, about 100-500, about 400-700 bp, about 500-800 bp, about 700-900 bp, about 800-1000 bp, or about 100-1000 bp.

The ends of DNA fragments can be polished (e.g., blunt-ended). The ends of DNA fragments can be polished by treatment with a polymerase. Polishing can involve removal of 3′ overhangs, fill-in of 5′ overhangs, or a combination thereof. The polymerase can be a proof-reading polymerase (e.g., comprising 3′ to 5′ exonuclease activity). The proofreading polymerase can be, e.g., a T4 DNA polymerase, Pol 1 Klenow fragment, or Pfu polymerase. Polishing can comprise removal of damaged nucleotides (e.g. abasic sites), using any means known in the art.

Ligation of an adaptor to a 3′ end of a DNA fragment can comprise formation of a bond between a 3′ OH group of the fragment and a 5′ phosphate of the adaptor. Therefore, removal of 5′ phosphates from DNA fragments can minimize aberrant ligation of two library members. Accordingly, in some embodiments, 5′ phosphates are removed from DNA fragments. In some embodiments, 5′ phosphates are removed from at least 50%, 55%, 60%, 65%, 70%, 75%, 80%, 85%, 90%, 95%, or greater than 95% of DNA fragments in a sample. In some embodiments, substantially all phosphate groups are removed from DNA fragments. In some embodiments, substantially all phosphates are removed from at least 50%, 55%, 60%, 65%, 70%, 75%, 80%, 85%, 90%, 95%, or greater than 95% of DNA fragments in a sample. Removal of phosphate groups from a DNA sample can be by any means known in the art. Removal of phosphate groups can comprise treating the sample with heat-labile phosphatase. In some embodiments, phosphate groups are not removed from the DNA sample. In some embodiments ligation of an adaptor to the 5′ end of the DNA fragment is performed.

### Denaturation

ssDNA can be prepared from dsDNA fragments prepared by any means in the art or as described herein, by denaturation into single strands. Denaturation of dsDNA can be by any means known in the art, including heat denaturation, incubation in basic pH, denaturation by urea or formaldehyde.

Heat denaturation can be achieved by heating a dsDNA sample to about 60 deg C. or above, about 65 deg C. or above, about 70 deg C. or above, about 75 deg C. or above, about 80 deg C. or above, about 85 deg C. or above, about 90 deg C. or above, about 95 deg C. or above, or about 98 deg C. or above. The dsDNA sample can be heated by any means known in the art, including, e.g., incubation in a water bath, a temperature controlled heat block, a thermal cycler. In some embodiments the sample is heated for 0.5, 1, 2, 3, 4, 5, 6, 7, 8, 9, 10, or more than 10 minutes.

Denaturation by incubation in basic pH can be achieved by, for example, incubation of a dsDNA sample in a solution comprising sodium hydroxide (NaOH) or potassium hydroxide (KOH). The solution can comprise about 1 mM NAOH, 2 mM NAOH, 5 mM NAOH, 10 mM NAOH, 20 mM NAOH, 40 mM NAOH, 60 mM NAOH, 80 mM NAOH, 100 mM NAOH, 0.2M NaOH, about 0.3M NaOH, about 0.4M NaOH, about 0.5M NaOH, about 0.6M NaOH, about 0.7M NaOH, about 0.8M NaOH, about 0.9M NaOH, about 1.0M NaOH, or greater than 1.0M NaOH. The solution can comprise about 1 mM KOH, 2 mM KOH, 5 mM KOH, 10 mM KOH, 20 mM KOH, 40 mM KOH, 60 mM KOH, 80 mM KOH, 100 mM KOH, 0.2M KOH, 0.5M KOH, 1M KOH, or greater than 1M KOH. In some embodiments, the dsDNA sample is incubated in NaOH or KOH for 0.5., 1, 2, 3, 4, 5, 6, 7, 8, 9, 10, 15, 20, 30, 40, 50, 60, or more than 60 minutes. The dsDNA can be incubated in Na-acetate following NaOH or KOH incubation.

Compounds like urea and formamide contain functional groups that can form H-bonds with the electronegative centers of the nucleotide bases. At high concentrations (e.g., 8M urea or 70% formamide) of the denaturant, the competition for H-bonds favors interactions between the denaturant and the N-bases rather than between complementary bases, thereby separating the two strands.

### Ligation of Primer-Docking Oligonucleotide.

A primer-docking oligonucleotide (pdo) can be ligated onto one end of a ssDNA fragment. The pdo can be ligated onto a 5′ end or a 3′ end. In some embodiments, the pdo is ligated onto a 3′ end of the ssDNA fragment.

The pdo generally comprises a sequence that acts as a template for annealing a primer. The sequence of the pdo can comprise a sequence that is at least 70% complementary to a portion or all of an adaptor sequence for coupling to an NGS platform (NGS adaptor). The pdo can comprise a sequence complementary or identical to 5, 6, 7, 8, 9, 10, 11, 12, 13, 14, 15, 20, or more than 20 contiguous nucleotides of an NGS adaptor. In some embodiments, the pdo does not comprise a sequence complementary to a portion or all of an NGS adaptor.

The pdo can be adenylated at a 5′ end. The pdo can be is conjugated to a capture moiety that is capable of forming a complex with a capture reagent. The capture moiety can be conjugated to the adaptor oligonucleotide by any means known in the art. Capture moiety/capture reagent pairs are known in the art. In some embodiments the capture reagent is avidin, streptavidin, or neutravidin and the capture moiety is biotin. In another embodiment the capture moiety/capture reagent pair is digoxigenin/wheat germ agglutinin.

Ligation of the pdo to the ssDNA fragment can be effected by an ATP-dependent ligase. In some embodiments, the ATP-dependent ligase is an RNA ligase. The RNA ligase can be an ATP dependent ligase. The RNA ligase can be an Rnl 1 or Rnl 2 family ligase. Generally, Rnl 1 family ligases can repair single-stranded breaks in tRNA. Exemplary Rnl 1 family ligases include, e.g., T4 RNA ligase, thermostable RNA ligase 1 from Thermus scitoductus bacteriophage TS2126 (CircLigase), or CircLigase II. These ligases generally catalyze the ATP-dependent formation of a phosphodiester bond between a nucleotide 3-OH nucleophile and a 5′ phosphate group. Generally, Rnl 2 family ligases can seal nicks in duplex RNAs. Exemplary Rnl 2 family ligases include, e.g., T4 RNA ligase 2. The RNA ligase can be an Archaeal RNA ligase, e.g., an archaeal RNA ligase from the thermophilic archaeon Methanobacterium thermoautotrophicum (MthRnl).

The ligation of the pdo's to the single-stranded nucleic acid fragment can comprise preparing a reaction mixture comprising an ssDNA fragment, a pdo, and ligase. In some embodiments the reaction mixture is heated to effect ligation of the adaptor oligonucleotides to the ss DNA fragments. In some embodiments the reaction mixture is heated to about 50 deg C., about 55 deg C., about 60 deg C., about 65 deg C., about 70 deg C., or above 70 deg C. In some embodiments the reaction mixture is heated to about 60-70 deg C. The reaction mixture can be heated for a sufficient time to effect ligation of the pdo to the ssDNA fragment. In some embodiments, the reaction mixture is heated for about 5 min, about 10 min, about 15 min, about 20 min, about 25 min, about 30 min, about 35 min, about 40 min, about 45 min, about 50 min, about 55 min, about 60 min, about 70 min, about 80 min, about 90 min, about 120 min, about 150 min, about 180 min, about 210 min, about 240 min, or more than 240 min.

In some embodiments the pdo's are present in the reaction mixture in a concentration that is greater than the concentration of ssDNA fragments in the mixture. In some embodiments, the pdo's are present at a concentration that is at least 10%, 20%, 30%, 40%, 60%, 60%, 70%, 80%, 90%, 100% or more than 100% greater than the concentration of ssDNA fragments in the mixture. The pdo's can be present at concentration that is at least 10-fold, 100-fold, 1000-fold, or 10000-fold greater than the concentration of ssDNA fragments in the mixture. The pdo's can be present at a final concentration of 0.1 uM, 0.5 uM, 1 uM, 10 uMor greater. In some embodiments the ligase is present in the reaction mixture at a saturating amount.

The reaction mixture can additionally comprise a high molecular weight inert molecule, e.g., PEG of MW 4000, 6000, or 8000. The inert molecule can be present in an amount that is about 0.5%, 1%, 2%, 3%, 4%, 5%, 7.5%, 10%, 12.5%, 15%, 17.5%, 20%, 25%, 30%, 35%, 40%, 45%, 50%, or greater than 50% weight/volume. In some embodiments, the inert molecular is present in an amount that is about 0.5-2%, about 1-5%, about 2-15%, about 10-20%, about 15-30%, about 20-50%, or more than 50% weight/volume.

After sufficient time has occurred to effect ligation of adaptors to the ss nucleic acid molecules, unreacted adaptors can be removed by any means known in the art, e.g., filtration by molecular weight cutoff, size exclusion chromatography, use of a spin column, selective precipitation with polyethylene glycol (PEG), selective precipitation with PEG onto a silica matrix, alcohol precipitation, sodium acetate precipitation, PEG and salt precipitation, or high stringency washing.

In some embodiments, the method further comprises capturing the ligated ssDNA fragment. Capturing of the ligated ssDNA fragment can occur prior to extension or subsequent to extension. The ligated ssDNA fragment can be captured onto a solid support. Capturing can involve the formation of a complex comprising a capture moiety conjugated to a pdo and a capture reagent. In some embodiments, the capture reagent is immobilized onto a solid support. In some embodiments the solid support comprises an excess of capture reagent as compared to the amount of ligated ssDNA comprising the capture moiety. In some embodiments the solid support comprises 5-fold, 10-fold, or 100-fold more available binding sites that the total number of ligated ssDNA fragments comprising the capture moiety.

### Extension

In some embodiments, a primer is hybridized to the ligated ssDNA fragment via the pdo. The primer can comprise a portion or entirety of an NGS adaptor sequence. Exemplary NGS adaptor sequences are described herein. In some embodiments, the primer is extended to create a duplex comprising the original ssDNA fragment and the extended primer, wherein the extended primer comprises a reverse complement of the original ssDNA fragment and an NGS adaptor sequence at one end. In some embodiments the NGS adaptor is at the 5′ end. Exemplary NGS adaptor sequences are described herein. In some embodiments, the NGS adaptor sequence comprises a sequence that is at least 70% identical to a surface-bound oligonucleotide of an NGS platform. In some embodiments, the NGS adaptor sequence comprises a sequence that is at least 70% complementary to a surface-bound oligonucleotide of an NGS platform. In some embodiments, the NGS adaptor sequence comprises a sequence that is at least 70% identical to a sequencing primer for use by an NGS platform. In some embodiments, the NGS adaptor sequence comprises a sequence that is at least 70% complementary to a sequencing primer for use by an NGS platform. Extension can be effected by a proofreading mesophilic DNA polymerase. Preferably, the polymerase is a thermophilic polymerase with 5′-3′ exonucleolytic/endonucleolytic (DNA polymerases I, II, III) or 3′-5′ exonucleolytic (family A or B DNA polymerases, DNA polymerase I, T4 DNA polymerase) activity. In some instances, the polymerase can have no exonuclease activity (Taq) In some cases, the polymerase effects linear amplification of the immobilized ligated fragment, creating a plurality of copies of the reverse complement of the immobilized ligated fragment. In other cases only one copy of the reverse complement is created. In some embodiments, the extended primer molecules are separated from the original ssDNA template (e.g., by denaturation as described herein). The extended primer molecules are free in solution while the original ssDNA template molecules remain immobilized to the solid support. The extended primer molecules can be easily harvested, resulting in a ssDNA library preparation in which most of the library members comprise an NGS adaptor. At least 50%, 60%, 70%, 80%, 90%, more than 90%, or substantially all of the library members can comprise an NGS adaptor.

An exemplary workflow for preparing a ssDNA library is outlined below.

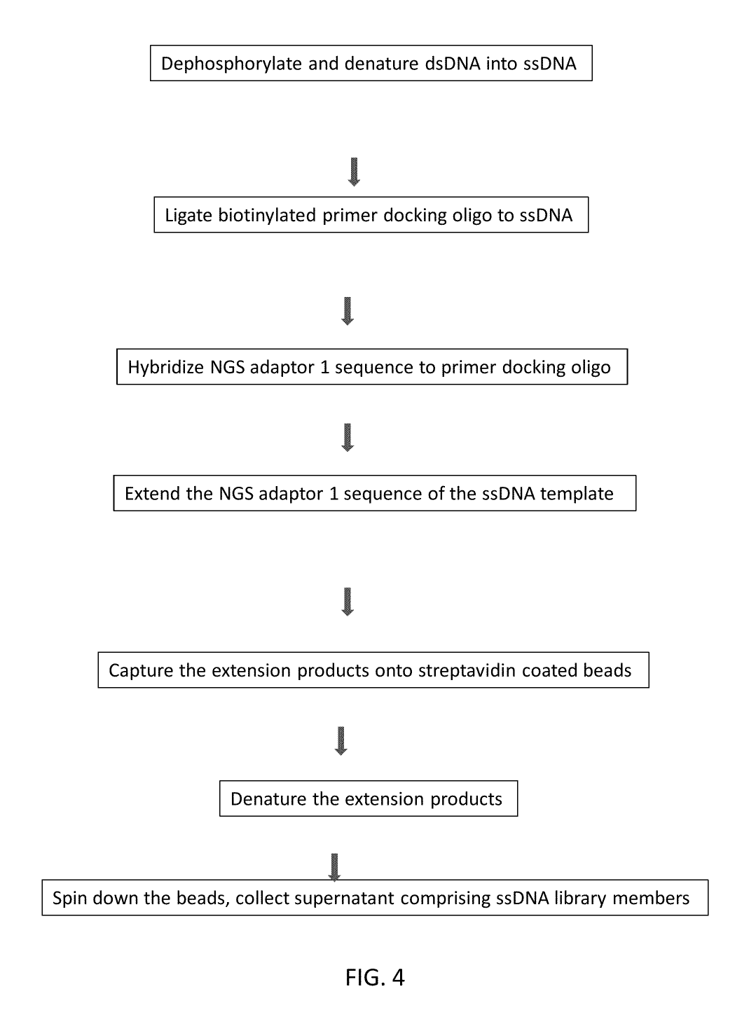

FIG. 4 depicts an exemplary embodiment of the method for preparing an ssDNA library from DNA isolated from a biological sample (e.g., a blood, plasma, urine, stool, mucosal sample). The DNA obtained is fragmented by enzymatic or mechanical means to 100-1000, but preferably 100-500 bp fragments. The DNA can be fragmented in situ. DNA can be fragmented from formalin-fixed paraffin-embedded (FFPE) tissues or circulating DNA. DNA can be isolated from FFPE and circulating by kits (Qiagen, Covaris). In some embodiments, the DNA is cDNA generated from RNA isolated from a biological sample from the same samples using random primed reverse transcription (RNaseH+) to generate randomly sized cDNA.

In step 1, fragmented DNA can be treated with a base exicision repair enzyme (Endo VIII, formamidopyrimidine DNA glycosylase (FPG)) to excise damaged bases that can interfere with polymerization. DNA can then be treated with a proof-reading polymerase (e.g. T4 DNA polymerase) to polish ends and replace damaged nucleotides (e.g. abasic sites) and a heat-labile phosphatase to remove all phosphate groups from DNA. The reaction mixture is heated to 80 deg C. for 10 min to inactivate the phosphatase and polymerase and denature double stranded DNA to single strands.

In step 2, a chemically or enzymatically phosphorylated pdo containing a 3′-end affinity tag (e.g. biotin) 12 to 50 bases in length can be ligated to the fragmented single-strand DNA library at a final concentration of 0.5 uM or greater with saturating amount of ATP-dependent RNA ligase (T4 RNA ligase, but preferably thermophillic such as CircLigase, CircLigase II) in the presence of 10-20% (w/v) polyethylene glycol of average molecular weight 4000, 6000, or 8000. The reaction is incubated for 1 hr @ 60-70C The pdo can comprise the following: (i) all, part or none of the sequence corresponding to a surface-bound oligonucleotide for Illumina flow cell cluster generation (ii) a 3′-end affinity group that is incapable of participating in the ligation reaction that is linked to the oligonucleotide at a sufficient distance (10 atoms or greater) to minimize steric hindrance of the interaction between the affinity ligand and the bound receptor.

The pdo can be adenlyated by any means known in the art. If an adenlyated adaptor is used, in some embodiments the ATP-dependent RNA ligase is not CircLigase or CircLigase II. The reaction is purified by size to remove unreacted adaptor. This can be achieved through the use of a microfiltration unit with a molecular size cutoff of 10K or 3K (e.g. microcon YM-10 or YM3, or nanosep omega). Alternatively, adaptor removal can be achieved through passage through a size exclusion desalting column (agarose, polyacrylamide) with a size exclusion cutoff of 10K or less, through the use of a spin column, through selective precipitation with PEG, alcohol or salt, high stringency washing, or denaturing gel electrophoresis.

In step 6 an oligonucleotide primer either fully complementary to the adaptor or partially complementary to the adaptor at its 3′-end, but fully possessing the sequence corresponding to the Illumina flow-cell oligonucleotides, is then used to create a reverse complement of the bound library using a proofreading mesophilic DNA polymerase. Preferably, a thermophilic polymerase with 5′-3′ exonucleolytic/endonucleolytic (DNA polymerase I) or 3′-5′ exonucleolytic (family A or B DNA polymerases, Vent, Phusion, Pfu and their variants) activity is used to permit linear amplification of the library.

In step 7 the recovered material is then bound to an affinity resin or support capable of binding to the 3′-end affinity tag in batch mode. The recovered material put into a pre-rinsed support in a 0.2 ml tube containing at least 10-fold excess and preferably 100-fold more available binding sites that the total number of tagged adaptor molecules.

In step 8 the supernatant consisting of copies of the bound library is then harvested and quantified.

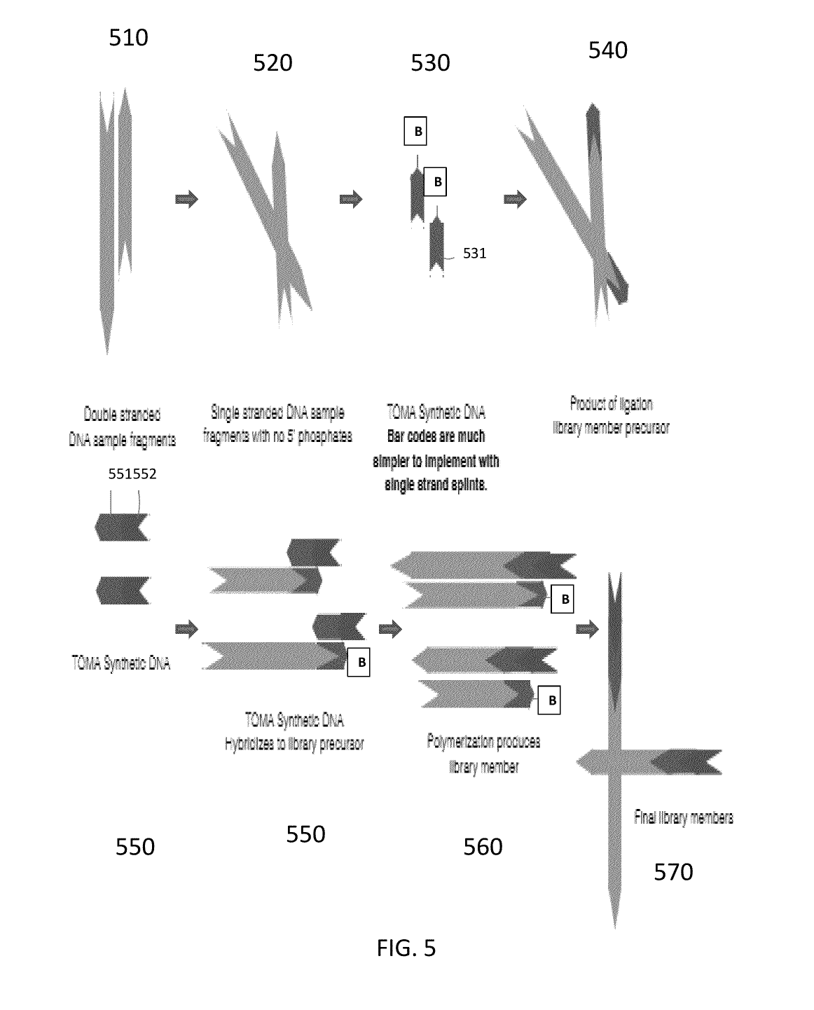

FIG. 5 is a depiction of an exemplary workflow as described in FIG. 4. In step 510 dsDNA is fragmented. In step 520 dsDNA fragments are dephosphorylated and heat-denatured into single strands. In step 530 biotinylated pdo's comprising a primer-docking sequence 531 are contacted with the ssDNA fragments. In step 540 the pdo's are ligated to the 3′ ends of the ssDNA fragments to create library member precursors. In step 550 primers comprising sequence complementary to the pdo 551 and adaptor sequence 552 are hybridized in step 560 to the ssDNA via the pdos. In step 560 the hybridized primers are extended along the template ssDNA fragments to create duplexes. The duplexes are immobilized onto a solid support (e.g., streptavidin coated beads). Heat denaturation releases the final library members into solution while retaining the original ssDNA fragment on the bead.

Alternative Embodiments of ssDNA Library Preparation.

In another aspect, the invention provides a method of preparing a ssDNA library, comprising denaturing dsDNA fragments into ssDNA, and ligating adaptor sequences to both ends of the ssDNA molecules. Methods of fragmenting dsDNA is described herein. Methods of denaturing dsDNA fragments are described herein.

The method can comprise ligating a first adaptor that comprises a sequence that is at least 70% complementary or identical to a first surface-bound oligonucleotide. The first surface-bound oligonucleotide can be an NGS platform-specific surface bound oligonucleotide. The first adaptor can comprise a sequence complementary or identical to 5, 6, 7, 8, 9, 10, 11, 12, 13, 14, 15, 20, or more than 20 contiguous nucleotides of the surface-bound oligonucleotide. The first adaptor can further comprise a sequence that is at least 70% complementary to a first sequencing primer. In some embodiments the first adaptor is ligated to a 3′ end of an ssDNA fragment using a method described herein or any method known in the art. In some embodiments, the ssDNA fragment lacks 5′ phosphate groups. In particular embodiments, the first adaptor is ligated to the 3′ end of the ssDNA fragment by an ATP-dependent ligase. In other embodiments, the first adaptor comprises a 3′ terminal blocking group. Generally, the 3′ terminal blocking group will prevent the formation of a covalent bond between the 3′ terminal base and another nucleotide. In some embodiments, the 3′ terminal blocking group is dideoxy-dNTP or biotin. The first adaptor can be 5′ adenylated. In some embodiments, the first adaptor is ligated to a 3′ end of an ssDNA fragment by an RNA ligase as described herein. The RNA ligase can be truncated or mutated RNA ligase 2 from T4 or Mth. The method can further comprises ligating a second adaptor sequence to a 5′ end of the ssDNA fragment. The second adaptor sequence can be distinct from the first adaptor sequence. The second adaptor sequence can comprise a sequence that is at least 70% complementary to a second surface-bound oligonucleotide. The second surface-bound oligonucleotide can be an NGS platform-specific surface bound oligonucleotide. The second adaptor can comprise a sequence complementary or identical to 5, 6, 7, 8, 9, 10, 11, 12, 13, 14, 15, 20, or more than 20 contiguous nucleotides of the surface-bound oligonucleotide. The second adaptor can further comprise a sequence that is at least 70% complementary to a second sequencing primer. In some embodiments the second adaptor is ligated to the ssDNA fragment using RNA ligase, e.g., CircLigase as described herein. In some embodiments, the first and second adaptor are both at least 70% complementary to the first and second surface-bound oligonucleotides. In other embodiments, the first and second adaptor are both at least 70% identical to the first and second surface-bound oligonucleotides.

The ssDNA library produced using methods described herein can be used for whole genome sequencing or targeted sequencing. In some embodiments, the ssDNA library produced using methods described herein are enriched for target polynucleotides of interest prior to sequencing.

### Target Enrichment

In another aspect, the invention provides a method for preparing a target-enriched DNA library. The method can involve hybridizing a target-selective oligonucleotide (TSO) to a single stranded DNA (ssDNA) fragment to create a hybridization product, and amplifying the hybridization product in a single round of amplification to create an extension strand.

The method of target enrichment is as described in US. Patent Application Pub. No. 20120157322, hereby incorporated by reference.

The hybridizing and amplifying can occur in a reaction mixture. The term “reaction mixture” as used herein generally refers to a mixture of components necessary to amplify at least one amplicon from nucleic acid template molecules. The mixture may comprise nucleotides (dNTPs), a polymerase and a target-selective oligonucleotide. In some embodiments, the mixture comprises a plurality of target-selective oligonucleotides. The mixture may further comprise a Tris buffer, a monovalent salt, and Mg2+. The concentration of each component is well known in the art and can be further optimized by an ordinary skilled artisan. The reaction mixture can also comprise additives including, but not limited to, non-specific background/blocking nucleic acids (e.g., salmon sperm DNA), biopreservatives (e.g. sodium azide), PCR enhancers (e.g. Betaine, Trehalose, etc.), and inhibitors (e.g. RNAse inhibitors). In some embodiments, a nucleic acid sample (e.g., a sample comprising an ssDNA fragment) is admixed with the reaction mixture. Accordingly, in some embodiments the reaction mixture further comprises a nucleic acid sample.

The ssDNA fragment can be a member of an ssDNA library. The ssDNA library can be prepared using a method as described herein. The ssDNA fragment can comprise a first single-stranded adaptor sequence located at a first end but not at a second end. In some embodiments, the first end is a 5′ end. In some embodiments, the TSO comprises a second single-stranded adaptor sequence located at a first end but not a second end. he first end can be a 5′ end. In some embodiments, the first adaptor sequence comprises a sequence that is at least 70% identical to a first surface-bound oligonucleotide. In some embodiments, the first adaptor sequence comprises a sequence that is at least 70% identical to a sequencing primer. In some embodiments the first adaptor further comprises a barcode sequence. In some embodiments, the second adaptor comprises a sequence that is at least 70% identical to a second surface-bound oligonucleotide. In some embodiments, the second adaptor comprises a sequence that is at least 70% identical to a sequencing primer

The target-selective oligonucleotide (tso) can be designed to at least partially hybridize to a target polynucleotide of interest. In some embodiments, the tso is designed to selectively hybridize to the target polynucleotide. The tso can be at least about 70%, 75%, 80%, 85%, 90%, 95%, or more than 95% complementary to a sequence in the target polynucleotide. In some embodiments, the tso is 100% complementary to a sequence in the target polynucleotide. The hybridization can result in a tso/target duplex with a Tm. The Tm of the tso/target duplex can be between 0-100 deg C., between 20-90 deg C., between 40-80 deg C., between 50-70 deg C., or between 55-65 deg C. The tso generally is sufficiently long to prime the synthesis of extension products in the presence of a polymerase. The exact length and composition of a tso can depend on many factors, including temperature of the annealing reaction, source and composition of the primer, and ratio of primer:probe concentration. The tso can be, for example, 8-50, 10-40, or 12-24 nucleotides in length.

### Amplification

The method can comprise amplification of the target in the reaction mixture. The amplification can be primed by a tso in a tso/target duplex. In some embodiments amplification is carried out utilizing a nucleic acid polymerase. The nucleic acid polymerase can be a DNA polymerase. In particular embodiments, the DNA polymerase is a thermostable DNA polymerase. The polymerase can be a member of A or B family DNA proofreading polymerases (Vent, Pfu, Phusion, and their variants), a DNA polymerase holoenzyme (DNA pol III holoenzyme), a Taq polymerase, or a combination thereof.

Amplification can be carried out as an automated process wherein the reaction mixture comprising template DNA is cycled through a denaturing step, a reporter probe and primer annealing step, and a synthesis step, whereby cleavage and displacement occurs simultaneously with primer-dependent template extension. The automated process may be carried out using a PCR thermal cycler. Commercially available thermal cycler systems include systems from Bio-Rad Laboratories, Life technologies, Perkin-Elmer, among others. In some embodiments, one cycle of amplification is performed.

Amplification of the tso/target duplex can result in an extension product comprising the original ssDNA fragment comprising the target sequence, and an extended strand comprising the second adaptor sequence, the tso, a reverse complement of the target sequence, and a reverse complement of the first adaptor sequence. If the first adaptor sequence of the original ssDNA fragment was 70% or more identical to a first surface-bound oligonucleotide, then the extended strand would comprise a first adaptor sequence that is 70% or more complementary to the first surface-bound oligonucleotide, and thereby would be hybridizable to the first surface-bound oligonucleotide. The extended strands, can comprise the target-enriched library.

The extension products in the reaction mixture can be denatured. The denatured extension products can be contacted with a surface immobilized thereon at least a first surface-bound oligonucleotide. In some embodiments, the extended strand is captured by the first surface-bound oligonucleotide, which can anneal to the first adaptor sequence on the extended strand.

The first surface-bound oligonucleotide can prime the extension of the captured extended strand. In some embodiments, extension of the captured extended strand results in a captured extension product. The captured extension product comprises the first surface bound oligonucleotide, the target sequence, and a second adaptor sequence that is at least 70% or more complementary to a second surface-bound oligonucleotide.

In some embodiments, the captured extension product hybridizes to the second surface-bound oligonucleotide, forming a bridge. In some embodiments, the bridge is amplified by bridge PCR. Bridge PCR methods can be carried out using methods known to the art.

### Kits for Library Preparation and Target Enrichment

Also provided are kits for practicing a method of library preparation as described herein or target-enrichment as described herein.

In one aspect, the invention provides kits for preparing a ssDNA library. In one embodiment, the kit comprises a pdo as described herein. In some embodiments, the kit comprises instructions, e.g., instructions for ligating a pdo to an ssDNA fragment. The kit can further comprise a ligase. The ligase can be an Rnl 1 or Rnl 2 family ligase, as described herein. The kit can further comprise a primer which can hybridize to the pdo. Primers hybridizable to the pdo are described herein. In some embodiments, the kit provides a solid support, e.g., a bead immobilized thereon a capture reagent. In some embodiments, the kit provides a polymerase for conducting an extension reaction. In some embodiments, the kit provides dNTPs for conducting an extension reaction.

In another embodiment, the kit comprises a first adaptor oligonucleotide that comprises sequence that is at least 70% complementary to a first support-bound oligonucleotide coupled to a sequencing platform, a second adaptor oligonucleotide that comprises a sequence that is distinct from the first adaptor, an RNA ligase, and instructions for use, e.g., instructions for practicing a method of the invention. In some embodiments, the first adaptor comprises a 3′ terminal blocking group that prevents the formation of a covalent bond between the 3′ terminal base and another nucleotide. 3′ terminal blocking groups are described herein. In some embodiments, the first is 5′ adenylated. In some embodiments, the first adaptor comprises a sequence that is at least 70% complementary to a sequencing primer. In some embodiments, the second adaptor comprises a sequence that is at least 70% complementary to a sequencing primer. In some embodiments, the second adaptor comprises a sequence that is at least 70% complementary to a second support-bound oligonucleotide coupled to a sequencing platform.

The invention provides kits for preparing a target-enriched DNA library. In some embodiments, the kit comprises a pdo, a ligase, a primer which can hybridize to the pdo, a solid support comprising a capture reagent, a polymerase, dNTPs, or any combination thereof. In some embodiments the kit further comprises a tso. The tso can be immobilized on a solid support coupled for sequencing on an NGS platform, as described in US Patent Application Pub No. 20120157322, hereby incorporated by reference.

In some embodiments, kits of the invention include a packaging material. As used herein, the term “packaging material” can refer to a physical structure housing the components of the kit. The packaging material can maintain sterility of the kit components, and can be made of material commonly used for such purposes (e.g., paper, corrugated fiber, glass, plastic, foil, ampules, etc.). Kits can also include a buffering agent, a preservative, or a protein/nucleic acid stabilizing agent.

### Sequencing

In some embodiments the target-enriched libraries are sequenced using any methods known in the art or as described herein. Sequencing can reveal the presence of mutations in one or more cancer-related genes in the set. In some embodiments a subset of 2, 3, 4 genes harboring the mutations are selected for further monitoring by assessment of cell-free DNA in a fluid sample isolated from the subject at later time points.

### Assessment of Cell-Free DNA Over Time

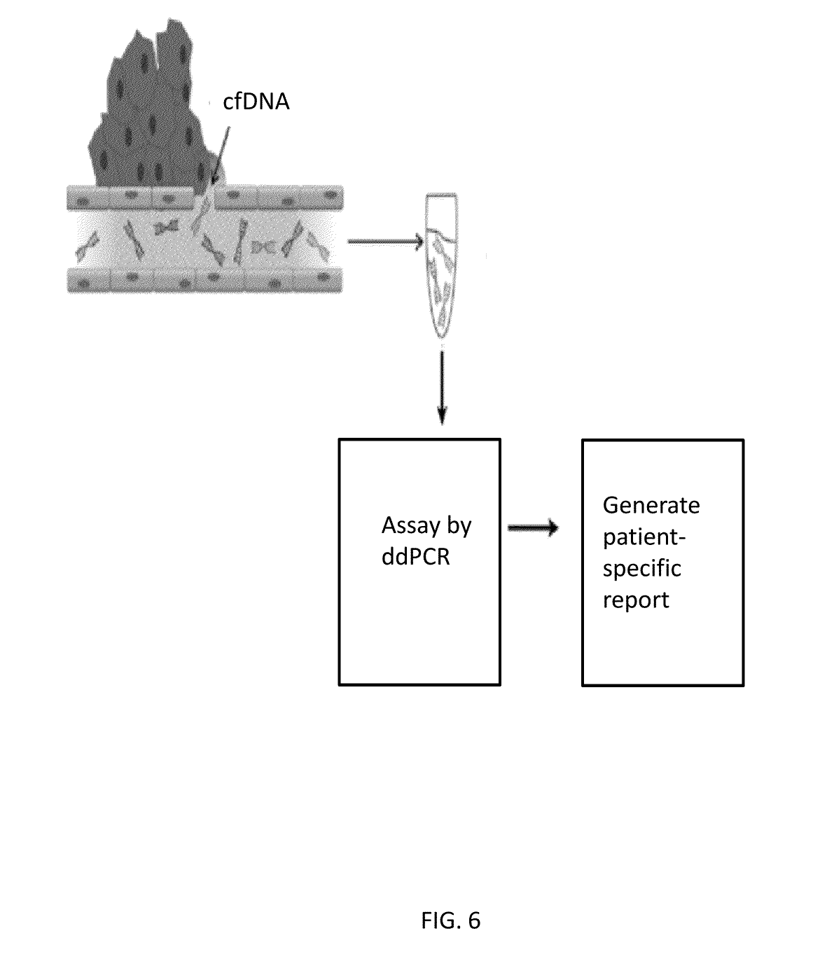

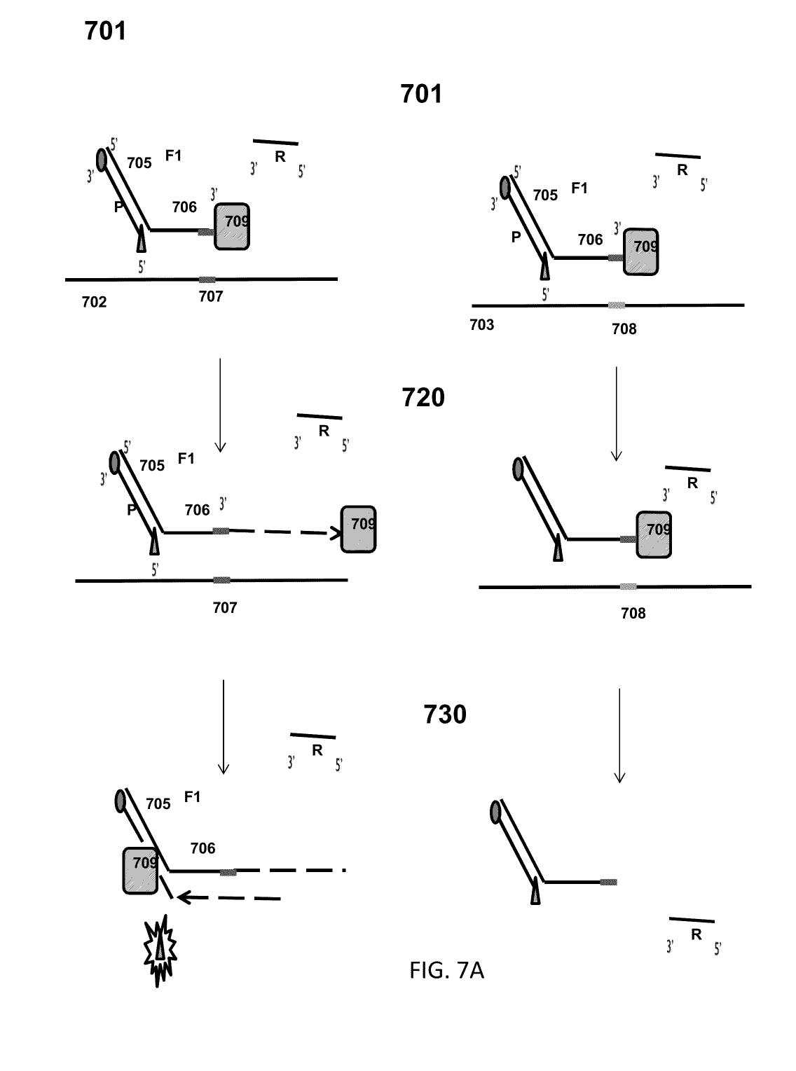

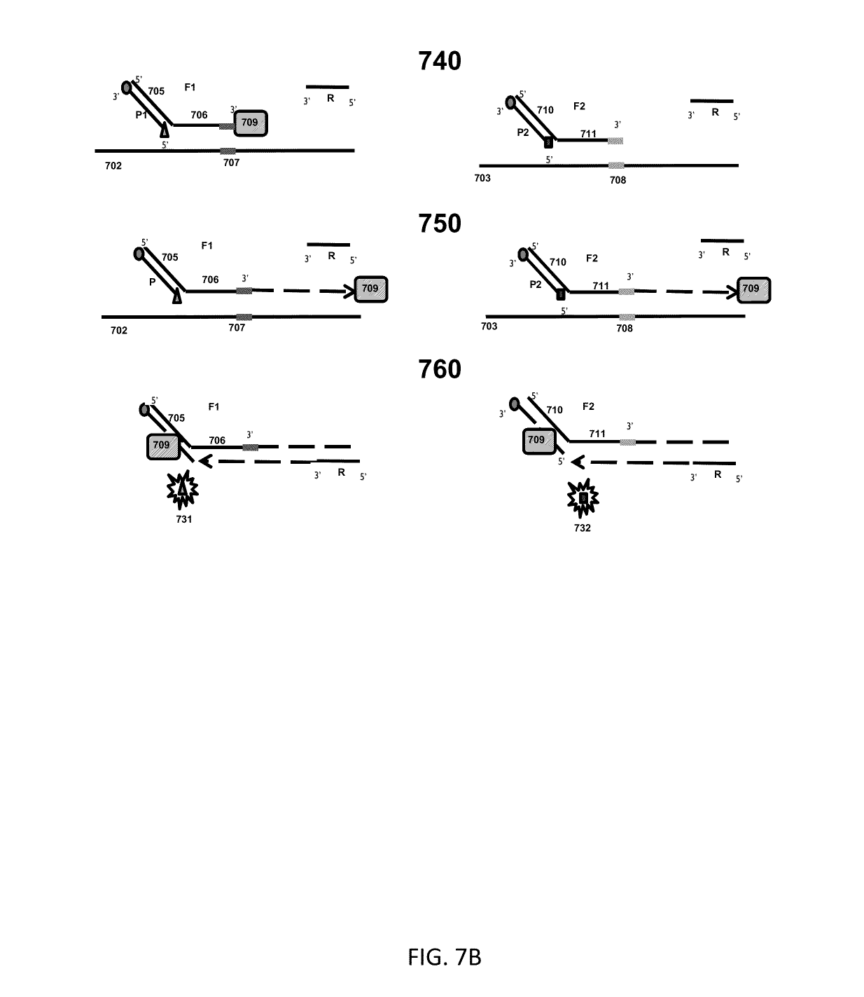

In some embodiments, assessment of cell free-DNA comprises detection and/or measurement of alleles of the subset of genes, as shown in FIG. 6. Detection of the alleles can be by any means known in the art or as described herein. The detection can be by methods as described in U.S. Pat. No. 5,538,848 (e.g., using a Taqman assay) or as described herein.

Accordingly, the present invention provides methods and kits for the sensitive detection of a mutation in a target polynucleotide. In some aspects, the methods and kits of the invention can be used for the discrimination of alleles in a target polynucleotide. For example, the invention provides methods and kits for the detection of mutant alleles in a background of high wild-type allelic ratio. For another example, the invention provides methods and kits for the detection of multiple alleles. In some embodiments, detection of an allele is enabled by release or activation of a detectable signal if the interrogated allele is present.

### Methods for Allele Detection

In some aspects, one or more methods of allele detection as described herein relate to the ability of an oligonucleotide primer to bind to a target polynucleotide region suspected of harboring the mutation. The oligonucleotide primer can partially overlay a locus of the suspected mutation. In some embodiments the oligonucleotide primer completely overlays the mutation. Accordingly, in some embodiments the mutation is small enough to be encompassed by an oligonucleotide primer. The mutation can be a single nucleotide polymorphism (SNP). The mutation can also comprise multiple nucleotide polymorphisms (e.g., double mutation or triple mutation). The mutation can be an insertion of one or more nucleotides. The mutation can be an insertion of 1, 2, 3, 4, 5, 6, 7, 8, 9, 10, 11, 12, 13, 14, 15, 16, 17, 18, 19, 20, 50, 100, 500, 1000, 10000, 100000, 1000000 nucleotides. The mutation can be an insertion of 1-5, 2-10, 5-15, or 10-20 nucleotides. In some embodiments, the mutation is a deletion of one or more nucleotides. The mutation can be a deletion of 1, 2, 3, 4, 5, 6, 7, 8, 9, 10, 11, 12, 13, 14, 15, 16, 17, 18, 19, 20 nucleotides. The mutation can be a deletion of 1-5, 2-10, 5-15, or 10-20 nucleotides. The mutation can be an inversion of two or more nucleotides. In some embodiments, 2, 3, 4, 5, or more nucleotides are inverted. In some embodiments, the mutation is a copy number variation (e.g., a copy number variation of a SNP or wild-type allele).

In one aspect, the invention provides a method of detecting a mutation in a target polynucleotide region, comprising the steps of: (a) contacting a nucleic acid sample with a reaction mixture comprising an oligonucleotide primer capable of hybridizing to the target polynucleotide region, wherein the oligonucleotide primer comprises a probe binding region and a template binding region that at least partially overlays a locus suspected of harboring the mutation and is capable of allele-specific extension by a polymerase; (b) extending the oligonucleotide primer to form an extension product; and (c) detecting the extension product, whereby the detecting the extension product indicates the presence of the mutation.

### Primers for Allele Detection

The oligonucleotide primer (e.g., a forward primer) can be designed to at least partially hybridize to a target polynucleotide suspected of harboring a mutation. In some embodiments, the template binding region of the forward primer is designed to selectively hybridize to the target polynucleotide. The hybridization can result in a forward primer/template duplex with a Tm. The Tm of the primer/template duplex can be between 0-100 deg C., between 20-90 deg C., between 40-80 deg C., between 50-70 deg C., or between 55-65 deg C. The template binding region of the forward primer can be 8-50, 10-40, or 12-24 nucleotides in length. The template binding region of the forward primer can be designed to at least partially overlay a particular locus suspected of harboring a mutation. The template binding region of the forward primer can, for example, overlay about at least 0.5%, 1%, 2%, 3%, 4%, 5%, 6%, 7%, 8%, 9%, 10%, 20%, 20%, 40%, 50%, 60%, 70%, 80%, 90%, or 100% of the locus suspected of harboring the mutation. The template binding region of the forward primer can overlay at least about 0.5-2%, 1-10%, 5-20%, 10-50%, 30-70%, 50-80%, 60-90%, or 80-100% of the locus suspected of harboring the mutation. The template binding region can be located at a 3′ region of the forward primer. In some embodiments, the region of the template binding region that overlays the locus is a 3′ terminal region. In some embodiments, the 3′ terminal region that overlays the mutation locus comprises 1, 2, 3, 4, 5, or more than 5 bases of the 3′-end of the template binding region. In some embodiments, the 3′ terminal base of the forward primer overlays the locus. In some embodiments, the 3′ terminal region of the forward primer is complementary to the interrogated allele. The 3′ terminal base of the forward primer may not complementary to the interrogated allele. In some embodiments, one or more mismatches is introduced into the 3′-region adjacent to the 3′-terminal base (e.g., n-1, n-2, n-3, etc.). These mismatches can be nucleotides or modified nucleotides that increase or decrease the impact of this mismatch on primer extension.

The template binding region can at least partially overlay with a locus that is suspected of having a copy number variation. In some embodiments, the template binding region of the forward primer can overlay at least about 0.5-2%, 1-10%, 5-20%, 10-50%, 30-70%, 50-80%, 60-90%, or 80-100% of the locus suspected of having a copy number variation.

The 3′ terminal region of the forward primer can comprise nucleotides linked by phosphorothioate linkages. In some embodiments, at least 2, 3, 4, 5, or more nucleotides at the 3′ terminal region of the forward primer are linked by phosphorothioate linkages.

Generally, the probe-binding region of the forward primer enables use of a reporter probe that is template independent. The probe-binding region can comprise a unique sequence or barcode that does not hybridize to the template nucleic acid. The probe-binding region can, for example, be designed to avoid significant sequence similarity or complementarity to known genomic sequences of an organism of interest. Such unique sequences can be randomly generated, e.g., by a computer readable medium, and selected by BLASTing against known nucleotide databases such as, e.g., EMBL, GenBank, or DDBJ. The barcode sequence can also be designed to avoid secondary structure. Tools for probe design are known in the art, and include, e.g., mFold, Primer Express. The probe-binding region can be 5-50, 6-40, or 7-30 nucleotides in length. The probe-binding region can be 1-20, 3-15, or 6-8 nucleotides away from the template binding region of the forward primer. The probe-binding region can be located 5′ of the template binding region.

In some embodiments, the method further comprises contacting the nucleic acid sample with a reverse primer. The reverse primer can be an oligonucleotide primer that corresponds to a region of template nucleic acid that is downstream of the forward primer. In some embodiments, the reverse primer is downstream of the interrogated allele. The reverse primer can bind to a reverse complement strand of the target polynucleotide. A forward/reverse primer pair can span a target region suspected of harboring a mutation. In some embodiments, the target region is 14-1000, 20-800, 40-600, 50-500, 70-300, 90-200, or 100-150 nucleotides long.

Primers or other oligonucleotides used in the present invention may further comprise a barcode sequence. Barcode sequences are described herein. In some embodiments, a barcode sequence encodes information relating to the identity of an interrogated allele, identity of a target polynucleotide or genomic locus, identity of a sample, a subject, or any combination thereof. A barcode sequence can be a portion of a primer, a reporter probe, or both. A barcode sequence may be at the 5′-end or 3′-end of an oligonucleotide, or may be located in any region of the oligonucleotide. A barcode sequence generally is not part of a template sequence. Barcode sequences may vary widely in size and composition; the following references provide guidance for selecting sets of barcode sequences appropriate for particular embodiments: Brenner, U.S. Pat. No. 5,635,400; Brenner et al, Proc. Natl. Acad. Sci., 97: 1665-1670 (2000); Shoemaker et al, Nature Genetics, 14: 450-456 (1996); Morris et al, European patent publication 0799897A1; Wallace, U.S. Pat. No. 5,981,179. A barcode sequence may have a length of about 4 to 36 nucleotides, about 6 to 30 nucleotides, or about 8 to 20 nucleotides.

Primers used in the present invention are generally sufficiently long to prime the synthesis of extension products in the presence of the agent for polymerization. The exact length and composition of a primer can depend on many factors, including temperature of the annealing reaction, source and composition of the primer, and ratio of primer:probe concentration. The primer length can be, for example, about 5-100, 10-50, or 20-30 nucleotides, although a primer may contain more or fewer nucleotides.

### Reporter Probes

In some embodiments, the reaction mixture further comprises a reporter probe. Generally, the reporter probe of the present invention is designed to produce a detectable signal indicating the presence of the interrogated allele.

The reporter probe can comprise a detectable moiety and a quencher moiety. The detectable moiety can be a dye. The dye can be a fluorescent dye, e.g., a fluorophore. The fluorescent dye can be a derivatized dye for attachment to the terminal 3′ carbon or terminal 5′ carbon of the probe via a linking moiety. The dye can be derivatized for attachment to the terminal 5′ carbon of the probe via a linking moiety. Quenching can involve a transfer of energy between the fluorophore and the quencher. The emission spectrum of the fluorophore and the absorption spectrum of the quencher can overlap. When the probe is intact, the fluorescent signal from the detectable moiety can be substantially suppressed by the quencher. Cleavage of the reporter probe, e.g., by hydrolysis, can separate the detectable moiety from the quencher moiety. The separation can enable the fluorescent moiety to produce a detectable fluorescent signal.

The reporter probes may be designed according to Livak et al., “Oligonucleotides with fluorescent dyes at opposite ends provide a quenched probe system useful for detecting PCR product and nucleic acid hybridization,” PCR Methods Appl. 1995 4: 357-362.

Reporter-quencher moiety pairs for particular probes can be selected according to, e.g., Pesce et at, editors, Fluorescence Spectroscopy (Marcel Dekker, New York, 1971); White et at, Fluorescence Analysis: A Practical Approach (Marcel Dekker, New York, 1970. Exemplary fluorescent and chromogenic molecules that may be used in reporter-quencher pairs, are described in, e.g. Berlman, Handbook of Fluorescence Sprectra of Aromatic Molecules, 2nd Edition (Academic Press, New York, 1971); Griffiths, Colour and Constitution of Organic Molecules (Academic Press, New York, 1976); Bishop, editor, Indicators (Pergamon Press, Oxford, 1972); Haugland, Handbook of Fluorescent Probes and Research Chemicals (Molecular Probes, Eugene, 1992); Pringsheim, Fluorescence and Phosphorescence (Interscience Publishers, New York, 1949).

A wide variety of reactive fluorescent reporter dyes can be used so long as they are quenched by a quencher dye of the invention. The fluorophore can be an aromatic or heteroaromatic compound. The fluorophore can be, for example, a pyrene, anthracene, naphthalene, acridine, stilbene, benzoxaazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Exemplary fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent reporters also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl)maleimide; cyanines, such as, e.g., indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno[2,3,4-ij: 5,6,7-i′j′]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3,6,7,12,13,16,17-octahydro-inner salt (TR or Texas Red); or BODIPYTM dyes. Exemplary fluorescent and quencher moieties are described in, e.g., WO/2005/049849.

As is known in the art, suitable quenchers are selected according to the fluorescer. Exemplary reporters and quenchers are further described in Anderson et al, U.S. Pat. No. 7,601,821.

Quenchers are also available from various commercial sources. Exemplary commercially available quenchers include, e.g., Black Hole Quenchers® from Biosearch Technologies and Iowa Black® or ZEN quenchers from Integrated DNA Technologies, Inc.

In some embodiments, the reporter probe comprises two quencher moieties. Exemplary probes comprising two quencher moieties include the Zen probes from Integrated DNA Technologies. Such probes comprise an internal quencher moiety that is located about 9 bases away from the detectable moiety, and generally reduce background signal associated with traditional reporter/quencher probes.

Detectable moieties and quencher moieties can be derivatized for covalent attachment to oligonucleotides via common reactive groups or linking moieties. Methods for derivatization of detectable and quencher moieties are described in, e.g., Ullman et al, U.S. Pat. No. 3,996,345; Khanna et al, U.S. Pat. No. 4,351,760; Eckstein, editor, Oligonucleotides and Analogues: A Practical Approach (IRL Press, Oxford, 1991); Zuckerman et al, Nucleic Acids Research, 15: 5305-5321 (1987) (3′ thiol group on oligonucleotide); Sharma et al, Nucleic Acids Research, 19:3019 (1991) (3′ sulfhydryl); Giusti et al, PCR Methods and Applications, 2:223-227 (1993) and Fung et al, U.S. Pat. No. 4,757,141 (5′ phosphoamino group via Aminolink™ II available from Applied Biosystems, Foster City, Calif.); Stabinsky, U.S. Pat. No. 4,739,044 (3′ aminoalkylphosphoryl group); Agrawal et al, Tetrahedron Letters, 31:1543-1546 (1990) (attachment via phosphoramidate linkages); Sproat et al, Nucleic Acids Research, 15:4837 (1987) (5′ mercapto group); Nelson et al, Nucleic Acids Research, 17:7187-7194 (1989) (3′ amino group).

In some embodiments, commercially available linking moieties can be attached to an oligonucleotide during synthesis, e g linking moieties available through Clontech Laboratories (Palo Alto, Calif.). By way of example only, rhodamine and fluorescein dyes can be derivatized with a phosphoramidite moiety for attachment to a 5′ hydroxyl of an oligonucleotide (see, e.g., Woo et al, U.S. Pat. No. 5,231,191; and Hobbs, Jr. U.S. Pat. No. 4,997,928)

In some embodiments, the detectable moiety produces a non-fluorescent signal. For example, any probe for which hydrolysis of the probe results in a detectable separation of a signal moiety from the detection probe-amplicon complex may be used. For example, release of the signal moiety may be detected electronically (e.g., as an electrode surface charge perturbation when a signal moiety is released from the detection probe/amplicon complex), by quantum dot sensing, by luminescence, or chemically (e.g., by a change in pH in a solution as a signal moiety is released into solution). Likewise, any probe that binds to a probe-binding region and for which a change in signal can be detected upon separation of a detectable moiety from a quencher moiety may be used. For example, molecular beacon probes, MGB probes, or other probes are contemplated for use in the invention. Molecular beacon probes are described in, e.g., U.S. Pat. Nos. 5,925,517 and 6,103,406. MGB probes are described in, e.g., U.S. Pat. No. 7,381,818.

The reporter probe can be designed to selectively hybridize to a probe-binding region as described herein. Accordingly, in some embodiments the reporter probe comprises a sequence that is complementary to at least a portion of the probe-binding region. The reporter probe can be 5-50, 6-40, or 7-30 nucleotides in length. The hybridization can result in a probe/primer duplex with a Tm. The Tm of the probe/primer duplex can be higher than the Tm of the primer/template duplex. The Tm of the probe/primer duplex can be 1, 2, 3, 4, 5, 6, 7, 8 9, 10, or more than 10 deg C. than the Tm of the primer/template duplex.

In some embodiments, the reporter probe selectively hybridizes to a sequence in the probe-binding region that is at least 2, 3, 4, 5, 6, 7, 8, 9, 10, 15, or 20 nucleotides apart from the template binding region of the primer.

The reporter probe can be present at a concentration that is higher than the concentration of the forward primer. The reporter probe can for example be present in a concentration that is, e.g., 1-10 fold or 1-5 fold higher than the concentration of the forward primer. The reporter probe can be present in a concentration that results in at least 50%, at least 60%, at least 70%, at least 80%, at least 90%, at least 95%, at least 96%, at least 97%, at least 98%, at least 99%, or about 100% of the forward primers occupied by the probe.

The primers and probes of the invention may be prepared by any suitable method. Methods for preparing oligonucleotides of specific sequence are known in the art, and include, for example, cloning and restriction of appropriate sequences and direct chemical synthesis. Chemical synthesis methods may include, for example, the phosphotriester method described by Narang et al., 1979, Methods in Enzymology 68:90, the phosphodiester method disclosed by Brown et al., 1979, Methods in Enzymology 68:109, the diethylphosphoramidate method disclosed in Beaucage et al., 1981, Tetrahedron Letters 22:1859, and the solid support method disclosed in U.S. Pat. No. 4,458,066.

In some embodiments, a forward primer comprising a template binding region and a probe-binding region can be prepared using two different oligonucleotides corresponding to the template binding region and probe binding region, respectively. The two oligonucleotides can be ligated enzymatically. Ligation can be by an RNA ligase. The RNA ligase can be an ATP dependent ligase. The RNA ligase can be an Rnl 1 family ligase. Generally, Rnl 1 family ligases can repair single-stranded breaks in tRNA. Exemplary Rnl 1 family ligases include, e.g., T4 RNA ligase, thermostable RNA ligase 1 from Thermus scitoductus bacteriophage TS2126 (CircLigase), or CircLigase II. Generally, Rnl 2 family ligases can seal nicks in duplex RNAs. Exemplary Rnl 2 family ligases include, e.g., T4 RNA ligase 2. The RNA ligase can be an Archaeal RNA ligase, e.g., an archaeal RNA ligase from the thermophilic archaeon Methanobacterium thermoautotrophicum (MthRnl). Ligation can also be effected by use of a splint oligonucleotide that spans the two oligonucleotides corresponding to the template binding and probe binding regions, respectively. In some embodiments, ligation using a splint oligonucleotide can comprise use of a T4 DNA ligase. Alternatively, ligation can be mediated by an ATP-independent ligase. Exemplary ATP-independent ligases include, e.g., RNA 3′-Phosphate Cyclase (RtcA), RNA ligase RtcB, or manufactured variants thereof. In some embodiments, ligation is performed indirectly through a two-step process, in which a template binding region is adenylated (e.g., adenylated chemically during synthesis or enzymatically using a ligase), and the adenylated template binding sequence is conjugated to the probe binding region.

Ligation can also be performed with “click chemistry.” Click chemistry is a concept that involves linking smaller subunits with simple chemistry. Smaller subunits can refer to small building blocks of larger molecules such as DNA bases, RNA nucleotides, linear or circularized DNA or RNA oligonucleotides. (3+2) cycloadditions between azide and alkyne groups which results in the formation of 1,2,3-triazole rings (e.g., copper-catalysed alkyne-azide coupling reaction) are generally considered typical click chemistry reactions. Other chemical ligation methods include the use of cyanogen bromide, phosphorothioate-iodoacetyl, and native ligation techniques where a C-terminal α-thioester is reacted in a chemoselective manner with an unprotected peptide containing an N-terminal Cys residue)

Primers and/or reporter probes can also be obtained from commercial sources such as Operon Technologies, Amersham Pharmacia Biotech, Sigma, IDT Technologies, and Life Technologies. The primers can have an identical melting temperature. The lengths of the primers can be extended or shortened at the 5′ end or the 3′ end to produce primers with desired melting temperatures. Also, the annealing position of each primer pair can be designed such that the sequence and, length of the primer pairs yield the desired melting temperature. The simplest equation for determining the melting temperature of primers smaller than 25 base pairs is the Wallace Rule (Td=2(A+T)+4(G+C)). Computer programs can also be used to design primers, including but not limited to Array Designer Software (Arrayit Inc.), Oligonucleotide Probe Sequence Design Software for Genetic Analysis (Olympus Optical Co.), NetPrimer, and DNAsis from Hitachi Software Engineering. The Tm (melting or annealing temperature) of each primer can be calculated using software programs such as Oligo Design, available from Invitrogen Corp.

The annealing temperature of the primers can be recalculated and increased after any cycle of amplification, including but not limited to cycle 1, 2, 3, 4, 5, cycles 6-10, cycles 10-15, cycles 15-20, cycles 20-25, cycles 25-30, cycles 30-35, or cycles 35-40. After the initial cycles of amplification, part of the primers may be incorporated into the products from each loci of interest, thus the TM can be recalculated based on the part of the primer incorporated into the product.

Reaction Mixture

The term “reaction mixture” as used herein generally refers to a mixture of components necessary to amplify at least one amplicon from nucleic acid template molecules. The mixture may comprise nucleotides (dNTPs), a polymerase and primers. The mixture may further comprise a Tris buffer, a monovalent salt, and Mg2+. The concentration of each component is well known in the art and can be further optimized by an ordinary skilled artisan. In some embodiments, the reaction mixture also comprises additives including, but not limited to, non-specific background/blocking nucleic acids (e.g., salmon sperm DNA), biopreservatives (e.g. sodium azide), PCR enhancers (e.g. Betaine, Trehalose, etc.), and inhibitors (e.g. RNAse inhibitors). In some embodiments, a nucleic acid sample is admixed with the reaction mixture. Accordingly, in some embodiments the reaction mixture further comprises a nucleic acid sample.

### Amplification

The method can comprise amplification of template nucleic acid in the reaction mixture. In some embodiments amplification is carried out utilizing a nucleic acid polymerase. The nucleic acid polymerase can be a DNA polymerase. The DNA polymerase can be a thermostable DNA polymerase.

Some aspects of the allele detection methods described herein relate to the ability of a DNA polymerase to separate a detectable moiety and quencher moiety in a reporter probe. Exemplary reporter probes are described herein. Separation of the detectable and quencher moiety can occur by cleavage of the reporter probe by the DNA polymerase. Cleavage of the reporter probe can occur by a 5′→3′ exonuclease activity of the DNA polymerase. Accordingly, in some embodiments, the DNA polymerase comprises 5′→3′ exonuclease activity. As used herein, “5′→3′ nuclease activity” or “5′ to 3′ nuclease activity” can refer to an activity of a template-specific nucleic acid polymerase whereby nucleotides are removed from the 5′ end of an oligonucleotide in a sequential manner. DNA polymerases with 5′→3′ exonuclease activity are known in the art and include, e.g., DNA polymerase isolated from Thermus aquaticus (Taq DNA polymerase).

Some aspects of the allele detection methods described herein further relate to the discriminative ability of a primer to be extended by a nucleic acid polymerase (e.g., a DNA polymerase) in an amplification step, depending on the presence or absence of a mismatch between the terminal 3′ base of the primer and its hybridized template polynucleotide. In cases wherein there is no mismatch between the terminal 3′ base of the primer and template nucleotide, extension of the primer by DNA polymerase can efficiently occur during an amplification reaction. In cases wherein there is a mismatch between the terminal 3′ base of the primer and template nucleotide (e.g., the bases are not complementary), extension of the primer by DNA polymerase does not occur. In some embodiments extension of the mismatched primer does not occur if the DNA polymerase lacks 3′→5′ exonuclease activity. 3′→5′ exonuclease activity, as used herein, generally refers to an activity of a DNA polymerase whereby the polymerase recognizes a mismatched basepair and moves backward by one base to excise the incorrect nucleotide. Accordingly, the DNA polymerase can lack 3′→5′ exonuclease activity. Exemplary DNA polymerases lacking 3′→5′ exonuclease activity include, but are not limited to BST DNA polymerase I, BST DNA polymerase I (large fragment), Tag polymerase, Streptococcus pneumoniae DNA polymerase I, Klenow Fragment (3′→5′ exo-), PyroPhage® 3173 DNA Polymerase, Exonuclease Minus (Exo-) (available from Lucigen), T4 DNA Polymerase, Exonuclease Minus (Lucigen). In some embodiments, the DNA polymerase is a recombinant DNA polymerase that has been engineered to lack exonuclease activity.

In other embodiments, extension of the mismatched primer by DNA polymerase does not occur wherein the DNA polymerase has 3′→5′ exonuclease activity. In particular embodiments, extension of the mismatched primer by DNA polymerase having 3′→5′ exonuclease activity does not occur if the 3′ terminal region of the mismatch primer comprises nucleotides linked by phosphorothioate linkages. Exemplary primers comprising nucleotides linked by phosphorothioate linkages are described herein.

In some embodiments, the PCR process is carried out as an automated process wherein the reaction mixture comprising template DNA is cycled through a denaturing step, a reporter probe and primer annealing step, and a synthesis step, whereby cleavage and displacement occurs simultaneously with primer-dependent template extension. The automated process may be carried out using a PCR thermal cycler. Commercially available thermal cycler systems include systems from Bio-Rad Laboratories, Life technologies, Perkin-Elmer, among others.

Repeated cycles of denaturation, primer/probe annealing, primer extension, and reporter probe cleavage can result in the exponential accumulation of detectable signal. Sufficient cycles are run to achieve detection of the detectable signal, which can be several orders of magnitude greater than background signal.

The present invention is compatible, however, with other amplification systems, such as the transcription amplification system, in which one of the PCR primers encodes a promoter that is used to make RNA copies of the target sequence. In similar fashion, the present invention can be used in a self-sustained sequence replication (3SR) system, in which a variety of enzymes are used to make RNA transcripts that are then used to make DNA copies, all at a single temperature. By incorporating a polymerase with 5′→3′ exonuclease activity into a ligase chain reaction (LCR) system, together with appropriate primer/probe sets, one can also employ the present invention to detect LCR products.

FIG. 7 depicts an exemplary embodiment of a method of the present invention. In a first step 701, a DNA sample comprising template DNA molecules 702 and 703 are contacted with a reaction mixture comprising dNTPs (not shown), a thermostable DNA polymerase 709 comprising 5′→3′ exonuclease activity and not comprising 3′→5′ exonuclease activity, a forward primer F1 comprising a probe-binding region 705 and a template binding region 706, and a reverse primer R. The 3′ terminal base of the forward primer F1 is complementary to a mutant allele 707 which resides on template molecule 702. By contrast, template molecule 703 has a wild-type allele 708 which is mismatched to the 3′ terminal base of forward primer F1. Also comprised in the reaction mixture is a reporter probe P which comprises a 5′ fluorescent moiety (triangle) and a 3′ quencher moiety (circle). In a first round of amplification (step 720), an annealing step is carried out wherein reporter probe P hybridizes to probe-binding region 705, resulting in a primer/reporter duplex P/F1. Additionally, F1 hybridizes to template molecules 702 and 703, resulting in complexes P/F1/102 and P/F1/103. During a synthesis step, DNA polymerase 709 promotes efficient extension of the P/F1/102 complex due to complementarity of the 3′ terminal base of F1 with mutant allele 707. The extension of F1 from template molecule 702 results in a chimeric extension product comprising the extended primer F1 and the hybridized reporter probe P. The extended primer F1 further comprises a primer binding site for reverse primer R. By contrast, extension of P/F1/103 does not occur because of a mismatch between wild-type allele 708 and the 3′ terminal base of F1. Accordingly, no chimeric extension product comprising the extended primer F1 and hybridized reporter probe P is produced from a template molecule containing the wild-type allele. In a second (and any subsequent round) of amplification (step 730), reverse primer R hybridizes to the chimeric extension product. DNA polymerase 709 promotes extension of reverse primer R, and the 5′→3′ exonuclease activity of polymerase 709 separates the fluorescent moiety from the quencher moiety, e.g., by hydrolysis, resulting in a detectable signal.

In some embodiments, a reaction mixture can comprise multiple primers and probes for multiplex detection. By way of example only, a reaction mixture can comprise a common reverse primer and two or more forward primers, wherein each of the forward primers hybridizes to the same region in the template polynucleotide but differs from the other forward primers in the 5′ probe-binding region, wherein each forward primer comprises a unique probe-binding region, and wherein the template binding region of each of the forward primers differs from the other forward primers in the 3′ terminal base, which is complementary to either a wild-type allele or to one or another mutant alleles. Accordingly, the reaction mixture can also comprise two or more different reporter probes, each probe having a sequence corresponding to one of the two or more unique probe-binding regions on the two or more forward primers and comprising a distinct detectable moiety that is detectably distinct from any other detectable moiety in the reaction mixture.

An exemplary embodiment of a multiplex assay detecting multiple alleles at a single locus is depicted in FIG. 7B. In a first step 740, a DNA sample comprising template DNA molecules 702 and 703 are contacted with a reaction mixture comprising dNTPs (not shown), a thermostable DNA polymerase 709 comprising 5′→3′ exonuclease activity and not 3′→5′ exonuclease activity, a forward primer F1 comprising a probe-binding region 705 and a template binding region 706, a forward primer F2 comprising a probe-binding region 710 and a template binding region 711. The template binding regions 706 and 711 are identical except for the 3′ terminal base, which in F1 is complementary to a mutant allele 707 which resides on template molecule 702 and in F2 is complementary to a wild-type allele 708 which resides on template molecule 703. Accordingly, there is a mismatch between the 3′ terminal base of 706 and wild-type allele 708, and a mismatch between the 3′ terminal base of 711 and mutant allele 707. Also comprised in the reaction mixture is reporter probe P1 which comprises a 5′ fluorescent moiety (triangle) and a 3′ quencher moiety (circle) and reporter probe P2 which comprises a spectrally distinct 5′ fluorescent moiety (square) and a 3′ quencher moiety (circle). The reporter probe P1 hybridizes to probe-binding region 705, resulting in a P1/F1 duplex, and reporter probe P2 hybridizes to probe-binding region 710, resulting in a P2/F2 duplex. In a first round of amplification (step 750), F1 and F2 hybridize to template molecules 702 and 703, which can result in P1/F1/702, P1/F1/703, P2/F2/702, and P2/F2/703 complexes. DNA polymerase 709 can promote efficient extension of P1/F1/702 and P2/F2/703, which can result in chimeric extension products comprising the extended primer F1 and the hybridized reporter probe P1 (F1-P1) and/or extended primer F2 and the hybridized reporter probe P2 (F2-P2), respectively. The extended primers F1-P1 and F2-P2 may each further comprise a primer binding site for reverse primer R. By contrast, no extension of P1/F1/703 or P2/F2/702 occurs due to the presence of a mismatch between the 3′ terminal base of the forward primers and the template DNA. Accordingly, no chimeric extension product comprising the extended primer F1 and hybridized reporter probe P2 or comprising extended primer F2 and hybridized reporter P1 is produced. In a second (and any subsequent round) of amplification (step 760), reverse primer R can hybridize to the chimeric extension products F1-P1 and F2-P2. DNA polymerase 709 can promote extension of reverse primer R, and the 5′→3′ exonuclease activity of polymerase 709 separates the fluorescent moiety from the quencher moiety of each probe P1 and P2, resulting in spectrally distinct signals 731 and 732.

By way of other example only, a reaction mixture can comprise a plurality of primer/probe sets, wherein each set comprises a plurality of forward primers for the detection of multiple alleles at a particular locus, each forward primer harboring a unique probe-binding sequence and a template binding region, the 3′ terminal base of the template binding region corresponding to an allele of the locus, a common reverse primer, and detectably distinct reporter probes specific for each forward primer in the set. Such a reaction mixture can be used for the multiplex detection of multiple alleles at a plurality of loci. Accordingly, in some embodiments the invention provides a method of detecting up to 2, 3, 4, 5, 6, 7, 8, 9, 10, 20, 30, 40, 50, 60, 70, 80, 90, 100 alleles in a single multiplex assay.

In some embodiments, a reaction mixture comprises a plurality of primer/probe sets, wherein each set comprises a forward primer harboring a unique probe-binding sequence and a template binding region, a reverse primer that binds to a region downstream of said forward primer, and a detectably distinct reporter probe specific for the forward primer. Such a reaction mixture can be used for the multiplex detection of multiple loci. Multiplex detection of multiple loci can be used to assay copy number variation. For example, a first locus can be a region suspected of having a copy number variation and second locus can be a region that is predicted to not have a copy number variation. Comparison of detectable signal corresponding to the first and second loci can be used to measure copy number variation.

The detectable signal can be monitored in real-time during each amplification cycle. As used herein, “real-time PCR” can refer to PCR methods wherein an amount of detectable signal is monitored with each cycle of PCR. In some embodiments, a cycle threshold (Ct) wherein a detectable signal reaches a detectable level is determined. Generally, the lower the Ct value, the greater the concentration of the interrogated allele. Generally, data is collected during the exponential growth (log) phase of PCR, wherein the quantity of the PCR product is directly proportional to the amount of template nucleic acid. Systems for real-time PCR are known in the art and include, e.g., the ABI 7700 and 7900HT Sequence Detection Systems (Applied Biosystems, Foster City, Calif.). The increase in signal during the exponential phase of PCR can provide a quantitative measurement of the amount of templates containing the mutant allele.

In other embodiments, the detectable signal is monitored after amplification cycles have terminated (e.g., endpoint detection).

### Partitioning/Digital PCR

The method also can comprise partitioning the reaction mixture and nucleic acid sample into discrete volumes prior to amplification. Discrete volumes can contain template nucleic acid molecules from a starting nucleic acid sample. The starting nucleic acid sample can be diluted such that discrete volumes contain on average less than one nucleic acid molecule. Partitions can contain no nucleic acid molecule. Partitions with no nucleic acids enable the use of Poisson statistics to determine original input DNA concentration. In particular embodiments, the starting nucleic acid sample is diluted such that discrete volumes contain on average 0.5 nucleic acid molecules or less. In some embodiments, discrete volumes can comprise a reaction mixture. Reaction mixtures are described herein. The method can comprise partitioning a nucleic acid sample into one set of discrete volumes, partitioning a reaction mixture into a second set of discrete volumes, and merging single discrete volumes from the first set with single discrete volumes from the second set to produce merged discrete volumes comprising a template nucleic acid molecule form and a reaction mixture. In other embodiments, the method comprises admixing a nucleic acid sample with a reaction mixture to produce an admixture, and partitioning the admixture into discrete volumes. Discrete volumes can be independently assayed for the detection of one or more alleles.

Specific methods for partitioning are not critical to the practice of the invention. For example, partitioning can be carried out by manual pipetting. In a particular example, reaction mixture and nucleic acid sample can be distributed to individual tubes or well by manual pipetting. In another example, robotic methods can be used for the partitioning step. Microfluidic methods can also be used for the partitioning step.

A discrete volume can be, e.g., a tube, a well, a perforated hole, a reaction chamber, or a droplet, such as a droplet of an aqueous phase dispersed in an immiscible liquid, such as described in U.S. Pat. No. 7,041,481. Discrete volumes can be arranged into arrays of discrete volumes. Exemplary arrays include the Open array digital PCR system by Life Technologies (described in http://tools.invitrogen.com/content/sfs/manuals/cms_{—}088717.pdf) and array systems by Fluidigm (www.fluidigm.com).

Partitioning a sample into small reaction volumes can confer many advantages. For example, the partitioning may enable the use of reduced amounts of reagents, thereby lowering the material cost of the analysis. By way of other example, partitioning can also improve sensitivity of detection. Without wishing to be bound by theory, partitioning of the reaction mixture and template DNA into discrete reaction volumes can give rare molecules greater proportional access to reaction reagents, thereby enhancing detection of rare molecules. For example, partitioning can enable the detection of a rare allele in a background of high wild-type allelic ratio. Accordingly, in some embodiments a reaction volume can be less than 1 ml, less than 500 microliters (ul), less than 100 ul, less than 10 ul, less than 1 ul, less than 0.5 ul, less than 0.1 ul, less than 50 nl, less than 10 nl, less than 1 nl, less than 0.1 nl, less than 0.01 nl, less than 0.001 nl, less than 0.0001 nl, less than 0.00001 nl, or less than 0.000001 nl. In some embodiments, a reaction volume can be 1-100 picoliters (pl), 50-500 μl, 0.1-10 nanoliters (nl), 1-100 nl, 50-500 nl, 0.1-10 microliters (ul), 5-100 ul, 100-1000 ul, or more than 1000 ul. In some embodiments, the reaction volumes are droplets. Without wishing to be bound by theory, the use of small droplets can enable the processing of large numbers of reactions in parallel. Accordingly, in some cases, the droplets have an average diameter of about, 0.000000000000001, 0.0000000000001, 0.00000000001, 0.000000001, 0.0000001, 0.000001, 0.00001, 0.0001, 0.001, 0.01, 0.05, 0.1, 1, 5, 10, 20, 30, 40, 50, 60, 70, 80, 100, 120, 130, 140, 150, 160, 180, 200, 300, 400, or 500 microns.

In some embodiments, the method comprises detection and/or measurement of an allele by digital PCR. The term “digital PCR”, as used herein, can refer to a PCR amplification which is carried out on a nominally single, selected template molecule, wherein a number of individual single molecules are each isolated into discrete reaction volumes. In some embodiments, a large number of reaction volumes are used to produce higher statistical significance. Generally, PCR amplification in a reaction volume containing a single template (such as, e.g., a well, chamber, bead, emulsion, etc.) can have either a negative result, e.g., no detectable signal if no starting molecule is present, or a positive result, e.g., a detectable signal, if the targeted starting molecule is present. By analyzing a number of reaction areas indicating a positive result, insight into the number of starting molecules can be obtained. Such an analysis can be used for measurement of an amount of wild-type or mutant alleles in a sample, or be used for a measurement of copy number variation of a locus in a sample.

In particular embodiments, the method comprises droplet digital PCR methods. “Droplet digital PCR” generally refers to digital PCR wherein the reaction volumes are droplets. The droplets provided herein can prevent mixing between reaction volumes.

The droplets described herein can include emulsion compositions. The term “emulsion”, as used herein, generally refers to a mixture of immiscible liquids (such as oil and an aqueous solution, e.g., water). In some embodiments, the emulsion comprise aqueous droplets within a continuous oil phase. In other embodiments, the emulsion comprises oil droplets within a continuous aqueous phase. The mixtures or emulsions described herein may be stable or unstable. In preferred embodiments, the emulsions are relatively stable.

In some embodiments the emulsions exhibit minimal coalescence. “Coalescence” refers to a process in which droplets combine to form progressively larger droplets. In some cases, less than 0.00001%, 0.00005%, 0.00010%, 0.00050%, 0.001%, 0.005%, 0.01%, 0.05%, 0.1%, 0.5%, 1%, 2%, 2.5%, 3%, 3.5%, 4%, 4.5%, 5%, 6%, 7%, 8%, 9%, or 10% of droplets exhibit coalescence. The emulsions may also exhibit limited flocculation, a process by which the dispersed phase comes out of suspension in flakes. In some cases, less than 0.00001%, 0.00005%, 0.00010%, 0.00050%, 0.001%, 0.005%, 0.01%, 0.05%, 0.1%, 0.5%, 1%, 2%, 2.5%, 3%, 3.5%, 4%, 4.5%, 5%, 6%, 7%, 8%, 9%, or 10% of droplets exhibit flocculation.

The droplets can either be monodisperse (e.g., of substantially similar size and dimensions) or polydisperse (e.g., of substantially variable size and dimensions. In some embodiments, the droplets are monodisperse droplets. In some cases, the droplets are generated such that the size of the droplets does not vary by more than plus or minus 5% of the average size of the droplets. In some cases, the droplets are generated such that the size of the droplets does not vary by more than plus or minus 2% of the average size of the droplets. In some cases, a droplet generator will generate a population of droplets from a single sample, wherein none of the droplets vary in size by more than plus or minus 0.1%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% of the average size of the total population of droplets.

In some embodiments, the present invention provides systems, devices, and methods for droplet generation. In some embodiments, microfluidic systems are configured to generate monodisperse droplets (see, e.g., Kiss et al. Anal Chem. 2008 Dec. 1; 80(23): 8975-8981). In some embodiments, the present invention provides micro fluidics systems for manipulating and/or partitioning samples.

In some embodiments, a microfluidics system comprises one or more of channels, valves, pumps, etc. (U.S. Pat. No. 7,842,248, herein incorporated by reference in its entirety). In some embodiments, a microfluidics system is a continuous-flow microfluidics system (see, e.g., Kopp et al., Science, vol. 280, pp. 1046-1048, 1998). In some embodiments, microarchitecture of the present invention includes, but is not limited to microchannels, microfluidic plates, fixed microchannels, networks of microchannels, internal pumps; external pumps, valves, centrifugal force elements, etc. In some embodiments, the microarchitecture of the present invention (e.g. droplet microactuator, microfluidics platform, and/or continuous-flow microfluidics) is complemented or supplemented with droplet manipulation techniques, including, but not limited to electrical (e.g., electrostatic actuation, dielectrophoresis), magnetic, thermal (e.g., thermal Marangoni effects, thermocapillary), mechanical (e.g., surface acoustic waves, micropumping, peristaltic), optical (e.g., opto-electrowetting, optical tweezers), and chemical means (e.g., chemical gradients). In some embodiments, a droplet microactuator is supplemented with a microfluidics platform (e.g. continuous flow components) and such combination approaches involving discrete droplet operations and microfluidics elements are within the scope of the invention.

In some embodiments, methods of the invention utilize a droplet microactuator. In some embodiments, a droplet microactuator is capable of effecting droplet manipulation and/or operations, such as, e.g., dispensing, splitting, transporting, merging, mixing, agitating. In some embodiments the invention employs droplet operation structures and techniques described in, e.g., U.S. Pat. Nos. 6,911,132, 6,773,566, and 6,565,727; U.S. patent application Ser. No. 11/343,284, and U.S. Patent Publication No. 20060254933.

Droplet digital PCR techniques enable a high density of discrete PCR amplification reactions in a single volume. In some embodiments, greater than 100,000, 500,000, 1,000,000, 1,500,000, 2,000,000, 2,500,000, 5,000,000, or 10,000,000 separate reactions may occur per ul.

### Detection

Fluorescence detection can be achieved using a variety of detector devices equipped with a module to generate excitation light that can be absorbed by a fluorescer, as well as a module to detect light emitted by the fluorescer. In some cases, samples (such as droplets) may be detected in bulk. For example, samples may be allocated in plastic tubes that are placed in a detector that measures bulk fluorescence from plastic tubes. The samples can be distributed in a monolayer. Monolayer distributed samples can be detected by scanning users high resolution scanners (e.g., microarray scanners, GenePix 4000B Microarray Scanner (Molecular Devices), SureScan Microarray Scanner (Agilent)). If the sample is distributed in multiple layers, the sample can be detected with confocal imaging (e.g., confocal microscopy, spinning-disk confocal microscopy, confocal laser scanning microscopy). In some cases, one or more samples (such as droplets) may be partitioned into one or more wells of a plate, such as a 96-well or 384-well plate, and fluorescence of individual wells may be detected using a fluorescence plate reader.

In some embodiments amplification of the droplets, e.g., in a thermal cycle results in the generation of one or more detectable signals in a number of droplets. During the amplification reaction, a droplet comprising a template DNA molecule containing an interrogated allele can exhibit an increase in fluorescence relative to droplets that do not contain an interrogated allele. Droplets can be processed individually and fluorescence data collected from the droplets. For example, data relating to fluorescent signals from spectrally distinct fluorophores may be collected from each droplet.

A number of commercial instruments are available for analysis of fluorescently labeled materials. For instance, the ABI Gene Analyzer can be used to analyze attomole quantities of DNA tagged with fluorophores such as ROX (6-carboxy-X-rhodamine), rhodamine-NHS, TAMRA (5/6-carboxytetramethyl rhodamine NHS), and FAM (5′-carboxyfluorescein NHS). These compounds are attached to the probe by an amide bond through a 5′-alkylamine on the probe. Attachment can also occur through phosphoramidite precursors (e.g., 2-methoxy-3-trifluoroacetyl-1,3,2-oxazaphosphacyclopentane or N-(3-(N′,N′-diisopropylaminomethoxyphosphinyloxy)propyl)-2,2,2-trifluoroacetamide) which is a method to conjugate amino-derivatized polymers, especially oligonucleotides. Other useful fluorophores include CNHS (7-amino-4-methyl-coumarin-3-acetic acid, succinimidyl ester), which can also be attached through an amide bond.

Following digital PCR, the number of positive samples having a particular allele and the number of positive samples having any other allele (e.g., a wild-type allele) can be counted. In some cases, quantitative determinations are made by measuring the fluorescence intensity of individual partitions, while in other cases, measurements are made by counting the number of partitions containing detectable signal. In some embodiments, control samples can be included to provide background measurements that can be subtracted from all the measurements to account for background fluorescence. In other embodiments, 1, 2, 3, 4, 5, 6, 7, 8, 9, 10, or more than 10 different colors can be used to detect and measure different alleles, such as by using fluorophores of different colors on different PCR primers matched to probes recognizing different sequences.

In another embodiment of the invention, detection of a hydrolyzed reporter probe can be accomplished using, for example, luminescence (e.g., using Yttrium or Berrilium conjugates of EDTA), time-resolved fluorescence spectroscopy, a technique in which fluorescence is monitored as a function of time after excitation, or fluorescence polarization, a technique to differentiate between large and small molecules based on molecular tumbling. Large molecules (e.g., intact labeled probe) tumble in solution much more slowly than small molecules. Upon linkage of a fluorescent moiety to the molecule of interest (e.g., the 5′ end of a labeled probe), this fluorescent moiety can be measured (and differentiated) based on molecular tumbling, thus differentiating between intact and digested probe. Detection may be measured directly during PCR or may be performed post PCR.

### Kits for Allele Detection

Also provided in the invention are kits for the detection of one or more alleles of a locus. Kits may include one or more oligonucleotide primers as described herein, wherein each of the primers is capable of selectively detecting an individual allele of a locus. Kits may also include one or more reporter probes, as described herein. Kits can include, for example, one or more primer/probe sets. Exemplary primer/probe sets are described herein. Kits may further comprise instructions for use of the one or more primer/probe sets, e.g., instructions for practicing a method of the invention. In some embodiments, the kit includes a packaging material. As used herein, the term “packaging material” can refer to a physical structure housing the components of the kit. The packaging material can maintain sterility of the kit components, and can be made of material commonly used for such purposes (e.g., paper, corrugated fiber, glass, plastic, foil, ampules, etc.). Kits can also include a buffering agent, a preservative, or a protein/nucleic acid stabilizing agent. Kits can also include other components of a reaction mixture as described herein. For example, kits may include one or more aliquots of thermostable DNA polymerase as described herein, and/or one or more aliquots of dNTPs. Kits can also include control samples of known amounts of template DNA molecules harboring the individual alleles of a locus. In some embodiments, the kit includes a negative control sample, e.g., a sample that does not contain DNA molecules harboring the individual alleles of a locus. In some embodiments, the kit includes a positive control sample, e.g., a sample containing known amounts of one or more of the individual alleles of a locus.

### Systems for Allele Detection

Also provided in the invention are systems for the detection of one or more alleles in a sample. The system can provide a reaction mixture as described herein. In some embodiments the reaction mixture is admixed with a DNA sample and comprises template DNA. In some embodiments, the system further provides a droplet generator, which partitions the template DNA molecules, probes, primers, and other reaction mixture components into multiple droplets within a water-in-oil emulsion. Examples of some droplet generators useful in the present disclosure are provided in International Application No. PCT/US2009/005317. The system can further provide a thermocycler, which reacts the droplets via, e.g., PCR, to allow amplification and generation of one or more detectable signals. During the amplification reaction, a droplet comprising a template DNA molecule containing an interrogated allele exhibits an increase in fluorescence relative to droplets that do not contain an interrogated allele. In some embodiments, the system further provides a droplet reader, which processes the droplets individually and collects fluorescence data from the droplets. The droplet reader may, for example, detect fluorescent signals from spectrally distinct fluorophores. In some cases, the droplet reader further comprises handling capabilities for droplet samples, with individual droplets entering the detector, undergoing detection, and then exiting the detector. For example, a flow cytometry device can be adapted for use in detecting fluorescence from droplet samples. In some cases, a microfluidic device equipped with pumps to control droplet movement is used to detect fluorescence from droplets in single file. In some cases, droplets are arrayed on a two-dimensional surface and a detector moves relative to the surface, detecting fluorescence at each position containing a single droplet. Exemplary droplet readers useful in the present disclosure are provided in International Application No. PCT/US2009/005317.

Other exemplary systems for use with the method of the invention is described, for example, PCT Patent Application Pubs. WO 2007/091228 (U.S. Ser. No. 12/092,261); WO 2007/091230 (U.S. Ser. No. 12/093,132); and WO 2008/038259. Systems useful in practicing the invention include, e.g., systems from Stokes Bio (www.stokebio.ie), Fluidigm (www.fluidigm.com), Bio-Rad Laboratories, (www.bio-rad.com) RainDance Technologies (www.raindancetechnologies.com), Microfluidic Systems (www.microfluidicsystems.com); Nanostream (www.nanostream.com); and Caliper Life Sciences (www.caliperls.com). Other exemplary systems suitable for use with the methods of the invention are described, for example, in Zhang et al. Nucleic Acids Res., 35(13):4223-4237 (2007), Wang et al., J. Micromech. Microeng., 15:1369-1377 (2005); Jia et al., 38:2143-2149 (2005); Kim et al., Biochem. Eng. J., 29:91-97; Chen et al., Anal. Chem., 77:658-666; Chen et al., Analyst, 130:931-940 (2005); Munchow et al., Expert Rev. Mol. Diagn., 5:613-620 (2005); and Charbert et al., Anal. Chem., 78:7722-7728 (2006); and Dorfman et al., Anal. Chem., 77:3700-3704 (2005).

In some embodiments, the system further comprises a computer which stores and processes data. A computer-executable logic may be employed to perform such functions as subtraction of background fluorescence, assignment of target and/or reference sequences, and quantification of the data. For example, the number of droplets containing fluorescence corresponding to the presence of a particular allele (e.g., a mutant allele) in the sample may be counted and compared to the number of droplets containing fluorescence corresponding to the presence of another allele at the locus (such as, e.g., a wild-type allele).

### Subject-Specific Report

In some embodiments, methods for assessing cancer as described herein further comprise generating a subject-specific report on the tumor profile. The tumor profile can comprise a mutational status of one or more genes in the set of genes sequenced. The method can further comprise generation a subject-specific report on mutational status of the subset of genes over time. The subject-specific report can comprise information on dynamics of the tumor over time, based on a change in the level of cell-free DNA harboring the mutations in the subset of genes over time. An increase over time of cell-free DNA harboring the mutations can indicate an increase in tumor or cancer burden. A decrease over time of cell-free DNA harboring the mutations can indicate a decrease in tumor or cancer burden.

In some embodiments, the report provides a stratification and/or annotation of treatment options for the subject, based on the subject's tumor-specific profile. The stratification and/or the annotation can be based on clinical information for the subject. The stratification can include ranking drug treatment options with a higher likelihood of efficacy higher than drug treatment options with a lower likelihood of efficacy or for which no information exists with regard to treating subjects with the determined status of the one or more molecular markers. The stratification can include indicating on the report one or more drug treatment options for which scientific information suggests the one or more drug treatment options will be efficacious in a subject, based on the status of one or more tumor-specific mutations from the subject. The stratification can include indicating on a report one or more drug treatment options for which some scientific information suggests the one or more drug treatment options will be efficacious in the subject, and some scientific information suggests the one or more drug treatment options will not be efficacious in the subject, based on the status of one or more tumor-specific mutations in the sample from the subject. The stratification can include indicating on a report one or more drug treatment options for which scientific information indicates the one or more drug treatment options will not be efficacious for the subject, based on the status of one or more tumor-specific mutations in the sample from the subject. The stratification can include color coding the listed drug treatment options on the report based on the rank of the predicted efficacy of the drug treatment options.

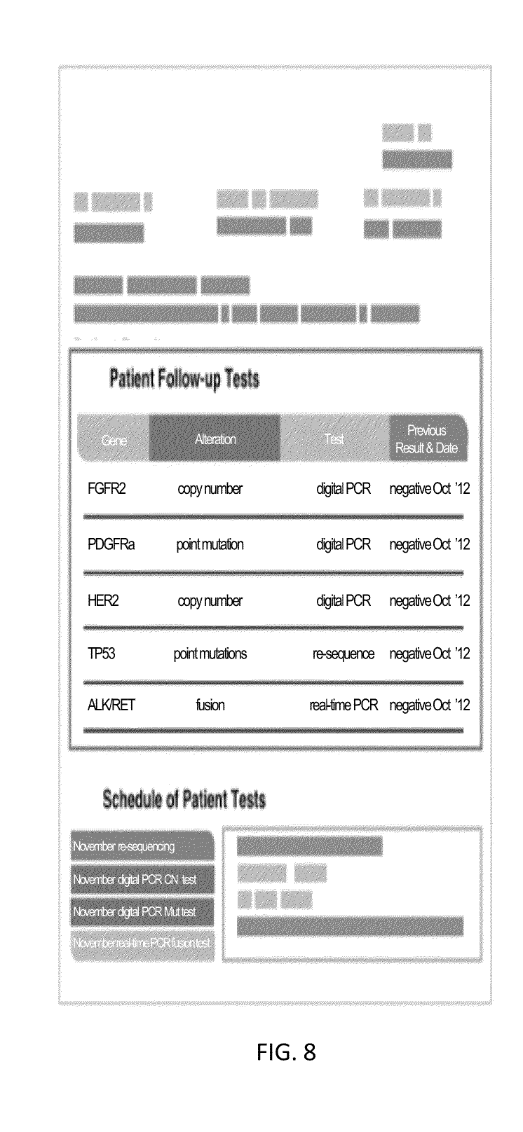

The annotation can include annotation a report for a condition in the NCCN Clinical Practice Guidelines in Oncology™ or the American Society of Clinical Oncology (ASCO) clinical practice guidelines. The annotation can include listing one or more FDA-approved drugs for off-label use, one or more drugs listed in a Centers for Medicare and Medicaid Services (CMS) anti-cancer treatment compendia, and/or one or more experimental drugs found in scientific literature, in the report. The annotation can include connecting a listed drug treatment option to a reference containing scientific information regarding the drug treatment option. The scientific information can be from a peer-reviewed article from a medical journal. The annotation can include using information provided by Ingenuity® Systems. The annotation can include providing a link to information on a clinical trial for a drug treatment option in the report. The annotation can include presenting information in a pop-up box or fly-over box near provided drug treatment options in an electronic based report. The annotation can include adding information to a report selected from the group consisting of one or more drug treatment options, scientific information concerning one or more drug treatment options, one or more links to scientific information regarding one or more drug treatment options, one or more links to citations for scientific information regarding one or more drug treatment options, and clinical trial information regarding one or more drug treatment options. An exemplary embodiment of a subject-specific report is depicted in FIG. 8.

### Computer Systems

In another aspect, the invention provides computer systems for the monitoring of a cancer, generating a subject report, and/or communicating the report to a caregiver. In some embodiments, the invention provides computer systems for determining prognosis or determining efficacy of a therapy for a cancer in a subject in need thereof. The computer system can provide a report communicating said prognosis or therapy efficacy for said cancer. In some embodiments, the computer system executes instructions contained in a computer-readable medium. In some embodiments, the processor is associated with one or more controllers, calculation units, and/or other units of a computer system, or implanted in firmware. In some embodiments, one or more steps of the method are implemented in hardware. In some embodiments, one or more steps of the method are implemented in software. Software routines may be stored in any computer readable memory unit such as flash memory, RAM, ROM, magnetic disk, laser disk, or other storage medium as described herein or known in the art. Software may be communicated to a computing device by any known communication method including, for example, over a communication channel such as a telephone line, the internet, a wireless connection, or by a transportable medium, such as a computer readable disk, flash drive, etc. The one or more steps of the methods described herein may be implemented as various operations, tools, blocks, modules and techniques which, in turn, may be implemented in firmware, hardware, software, or any combination of firmware, hardware, and software. When implemented in hardware, some or all of the blocks, operations, techniques, etc. may be implemented in, for example, an application specific integrated circuit (ASIC), custom integrated circuit (IC), field programmable logic array (FPGA), or programmable logic array (PLA).

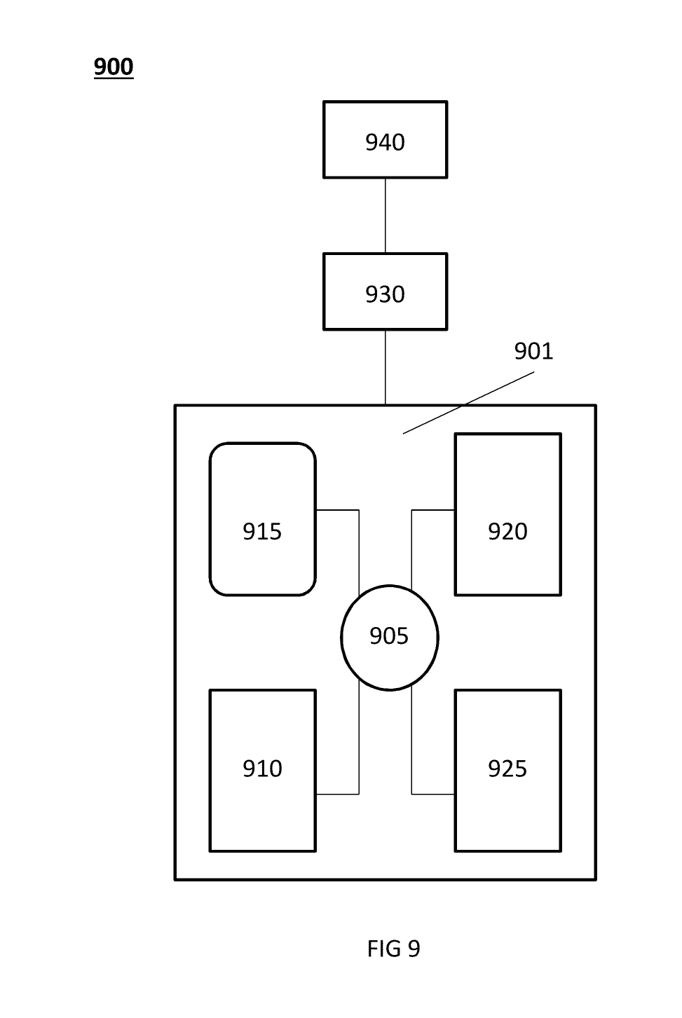

FIG. 9 depicts a computer system 900 adapted to enable a user to detect, analyze, and process patient data. The system 900 includes a central computer server 901 that is programmed to implement exemplary methods described herein. The server 901 includes a central processing unit (CPU, also “processor”) 905 which can be a single core processor, a multi core processor, or plurality of processors for parallel processing. The server 901 also includes memory 910 (e.g. random access memory, read-only memory, flash memory); electronic storage unit 915 (e.g. hard disk); communications interface 920 (e.g. network adaptor) for communicating with one or more other systems; and peripheral devices 925 which may include cache, other memory, data storage, and/or electronic display adaptors. The memory 910, storage unit 915, interface 920, and peripheral devices 925 are in communication with the processor 905 through a communications bus (solid lines), such as a motherboard. The storage unit 915 can be a data storage unit for storing data. The server 901 is operatively coupled to a computer network (“network”) 930 with the aid of the communications interface 920. The network 930 can be the Internet, an intranet and/or an extranet, an intranet and/or extranet that is in communication with the Internet, a telecommunication or data network. The network 930 in some cases, with the aid of the server 901, can implement a peer-to-peer network, which may enable devices coupled to the server 901 to behave as a client or a server.

The storage unit 915 can store files, such as subject reports, and/or communications with the caregiver, sequencing data, data about individuals, or any aspect of data associated with the invention.

The server can communicate with one or more remote computer systems through the network 930. The one or more remote computer systems may be, for example, personal computers, laptops, tablets, telephones, Smart phones, or personal digital assistants.

In some situations the system 900 includes a single server 901. In other situations, the system includes multiple servers in communication with one another through an intranet, extranet and/or the Internet.

The server 901 can be adapted to store sequencing information, or patient information, such as, for example, polymorphisms, mutations, patient history and demographic data and/or other information of potential relevance. Such information can be stored on the storage unit 915 or the server 901 and such data can be transmitted through a network.

Methods as described herein can be implemented by way of machine (or computer processor) executable code (or software) stored on an electronic storage location of the server 901, such as, for example, on the memory 910, or electronic storage unit 915. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910. Alternatively, the code can be executed on a second computer system 940.

Aspects of the systems and methods provided herein, such as the server 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tantible “storage” media, terms such as computer or machine “readable medium” can refer to any medium that participates in providing instructions to a processor for execution.

Hence, a machine readable medium, such as computer-executable code, may take many forms, including but not limited to, tangible storage medium, a carrier wave medium, or physical transmission medium. Non-volatile storage media can include, for example, optical or magnetic disks, such as any of the storage devices in any computer(s) or the like, such may be used to implement the system. Tangible transmission media can include: coaxial cables, copper wires, and fiber optics (including the wires that comprise a bus within a computer system). Carrier-wave transmission media may take the form of electric or electromagnetic signals, or acoustic or light waves such as those generated during radio frequency (RF) and infrared (IR) data communications. Common forms of computer-readable media therefore include, for example: a floppy disk, a flexible disk, hard disk, magnetic tape, any other magnetic medium, a CD-ROM, DVD, DVD-ROM, any other optical medium, punch cards, paper tame, any other physical storage medium with patterns of holes, a RAM, a ROM, a PROM and EPROM, a FLASH-EPROM, any other memory chip or cartridge, a carrier wave transporting data or instructions, cables, or links transporting such carrier wave, or any other medium from which a computer may read programming code and/or data. Many of these forms of computer readable media may be involved in carrying one or more sequences of one or more instructions to a processor for execution.

The results of monitoring of a cancer, generating a subject report, and/or communicating the report to a caregiver can be presented to a user with the aid of a user interface, such as a graphical user interface.

A computer system may be used for one or more steps, including, e.g., sample collection, sample processing, sequencing, allele detection, receiving patient history or medical records, receiving and storing measurement data regarding a detected level of tumor-specific mutations in a subject or sample obtained from a subject, analyzing said measurement data determine a diagnosis, prognosis, or therapeutic efficacy, generating a report, and reporting results to a receiver.

A client-server and/or relational database architecture can be used in the invention. In general, a client-server architecture is a network architecture in which each computer or process on the network is either a client or a server. Server computers can be powerful computers dedicated to managing disk drives (file servers), printers (print servers), or network traffic (network servers). Client computers can include PCs (personal computers) or workstations on which users run applications, as well as example output devices as disclosed herein. Client computers can rely on server computers for resources, such as files, devices, and even processing power. The server computer handles all of the database functionality. The client computer can have software that handles front-end data management and receive data input from users.

After performing a calculation, a processor can provide the output, such as from a calculation, back to, for example, the input device or storage unit, to another storage unit of the same or different computer system, or to an output device. Output from the processor can be displayed by a data display, e.g., a display screen (for example, a monitor or a screen on a digital device), a print-out, a data signal (for example, a packet), a graphical user interface (for example, a webpage), an alarm (for example, a flashing light or a sound), or a combination of any of the above. In an embodiment, an output is transmitted over a network (for example, a wireless network) to an output device. The output device can be used by a user to receive the output from the data-processing computer system. After an output has been received by a user, the user can determine a course of action, or can carry out a course of action, such as a medical treatment when the user is medical personnel. In some embodiments, an output device is the same device as the input device. Example output devices include, but are not limited to, a telephone, a wireless telephone, a mobile phone, a PDA, a flash memory drive, a light source, a sound generator, a fax machine, a computer, a computer monitor, a printer, an iPod, and a webpage. The user station may be in communication with a printer or a display monitor to output the information processed by the server. Such displays, output devices, and user stations can be used to provide an alert to the subject or to a caregiver thereof.

Data relating to the present disclosure can be transmitted over a network or connections for reception and/or review by a receiver. The receiver can be but is not limited to the subject to whom the report pertains; or to a caregiver thereof, e.g., a health care provider, manager, other healthcare professional, or other caretaker; a person or entity that performed and/or ordered the genotyping analysis; a genetic counselor. The receiver can also be a local or remote system for storing such reports (e.g. servers or other systems of a “cloud computing” architecture). In one embodiment, a computer-readable medium includes a medium suitable for transmission of a result of an analysis of a biological sample.

An exemplary embodiment of a subject-specific report is depicted in FIG. 8. The computer system can comprise a user accessible module which enables the ability for clinicians to request a service be performed. Clinicians can enter patient demographic and medical history information into the computer system. The computer system can process the entered information and create a barcode label that can be applied to the sample being analyzed. The barcoded-sample be sent for analysis to a third party analyzer. The barcoded information would be inaccessible to the third party analyzer to maintain accountability with The Health Insurance Portability and Accountability Act (HIPAA) compliancy. Information that can be anonymized can be accessible to the third party analyzer. The barcode can be used to track the progression of the sample through the analysis workflow resulting in the generation of an encrypted final report. The encrypted final report can be decrypted and made accessible to the clinician who originally entered the sample information.

### EXAMPLES

### Example 1

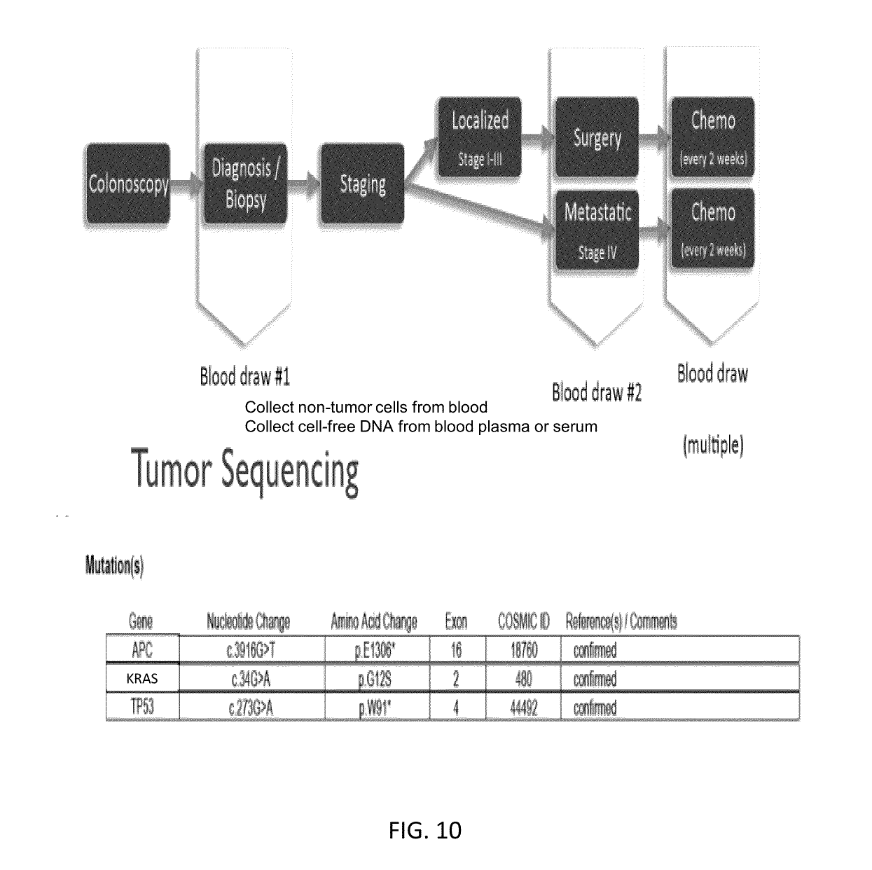

FIG. 10 depicts a method used to assess a cancer in a subject. A subject had a colonoscopy and is discovered to harbor a colon tumor. A tumor biopsy and blood draw were collected from the subject at time point 0, and are used to aid in the diagnosis of colon cancer in the subject. The tumor and normal cells from the first blood draw were sequenced. Sequencing revealed the presence of three mutations in the subject's tumor. The mutations were point mutations in the APC, KRAS, and TP53 genes. The stage of the subject's cancer was determined. The subject underwent a first treatment (surgery) to remove the tumor. Upon the first treatment, a second blood draw was performed. It was determined that the subject's tumor had metastasized. The subject was administered as second therapy (chemotherapy) to manage the cancer. Subsequent blood draws are performed to assay the mutational status of the three genes in cell-free DNA from the blood.

### Example 2

### Validation Assay for a Tumor-Specific Mutation in the Subject with Colon Cancer

NCI-H1573 (CRL-5877) cell lines harboring the KRAS G12A mutation (mu) were obtained as frozen stocks from the American Type Culture Collection (ATCC). Genomic DNA (gDNA) was prepared from cell line material using a commercially available kit (DNeasy Blood & Tissue kit, QIAGEN), according to the manufacturer's suggested protocol. Estimates of DNA concentration were obtained spectrophotometrically by measuring the OD₂₆₀ (NanoDrop 1000, Thermo Fisher Scientific Inc.).

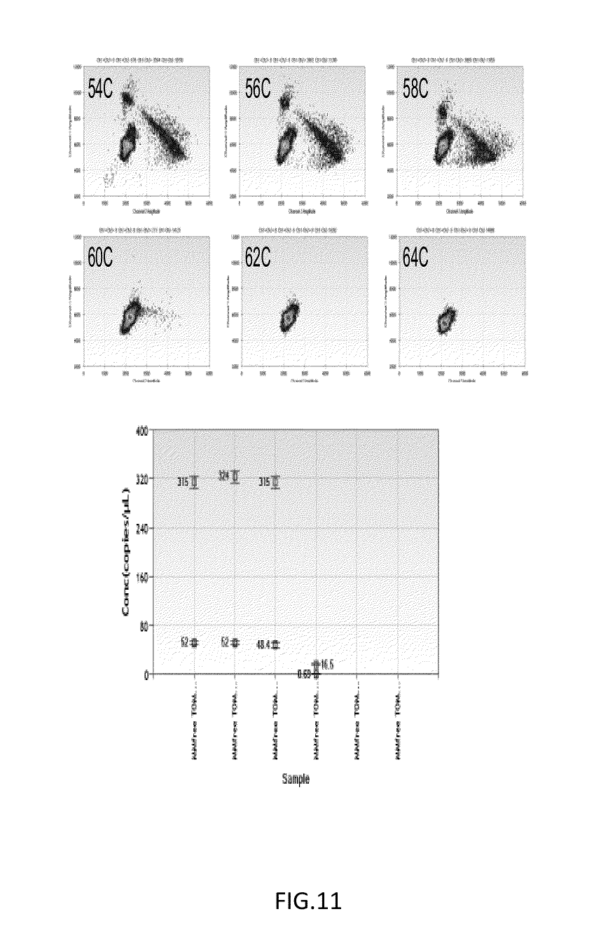

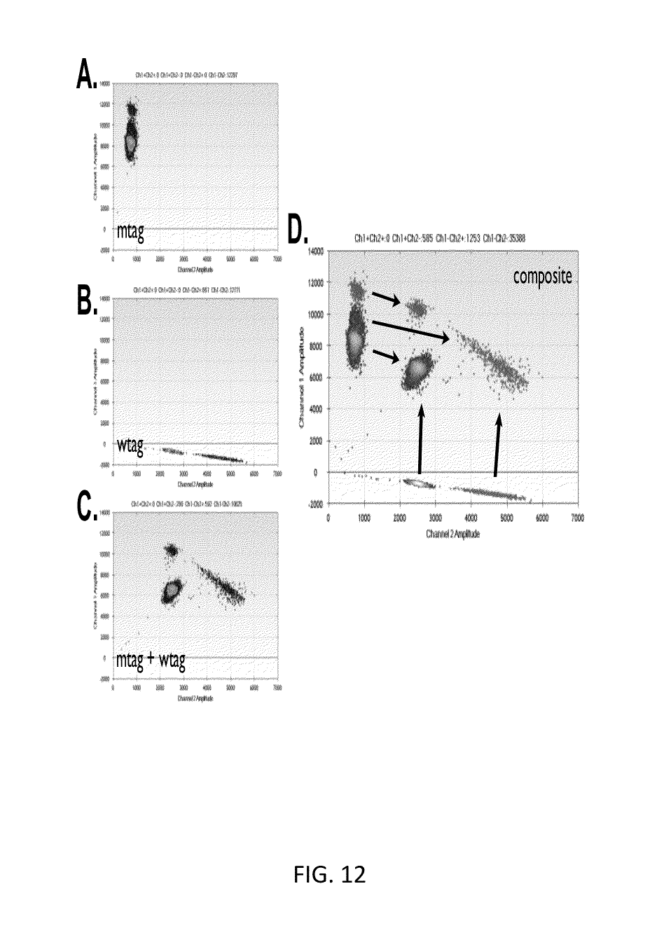

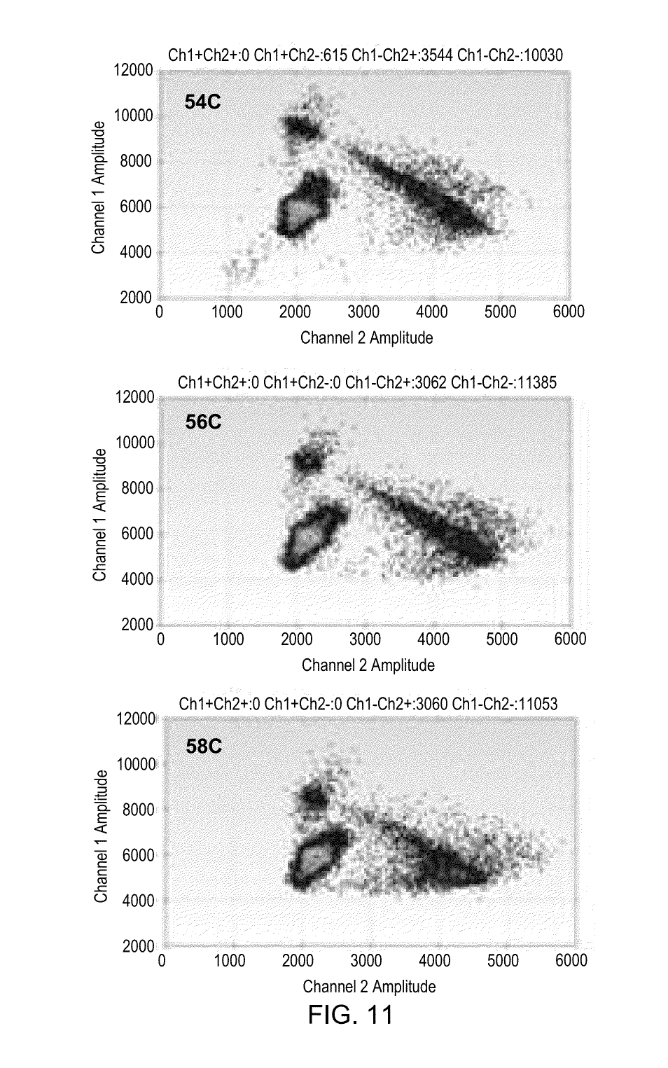

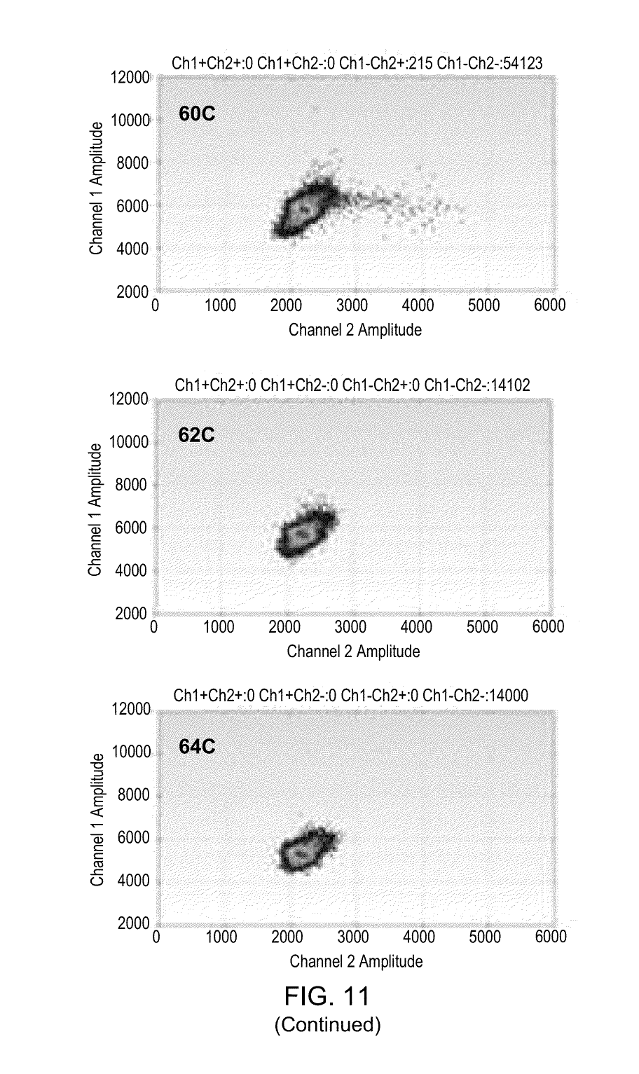

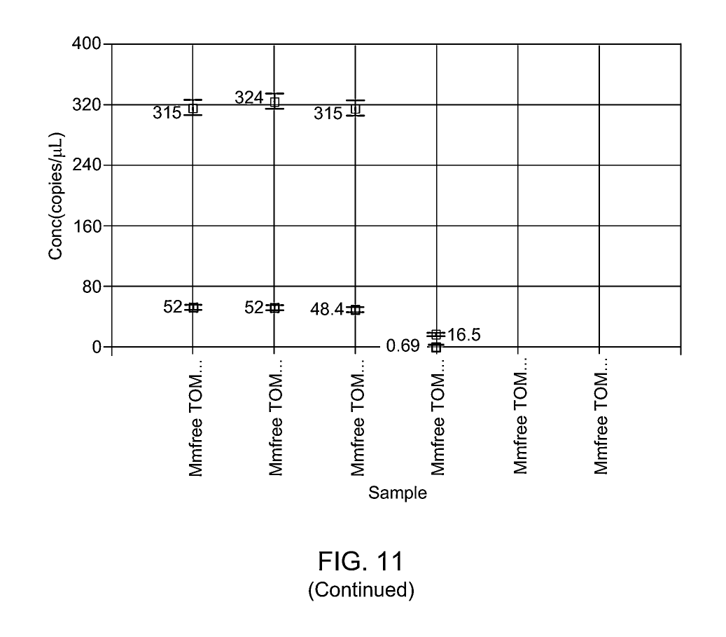

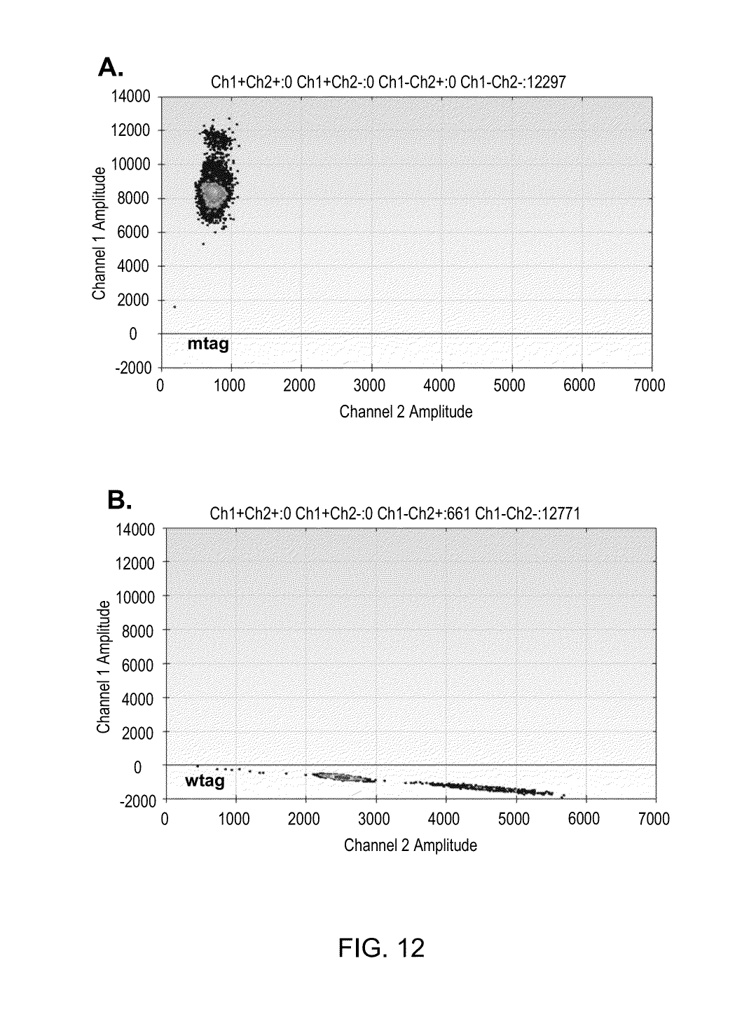

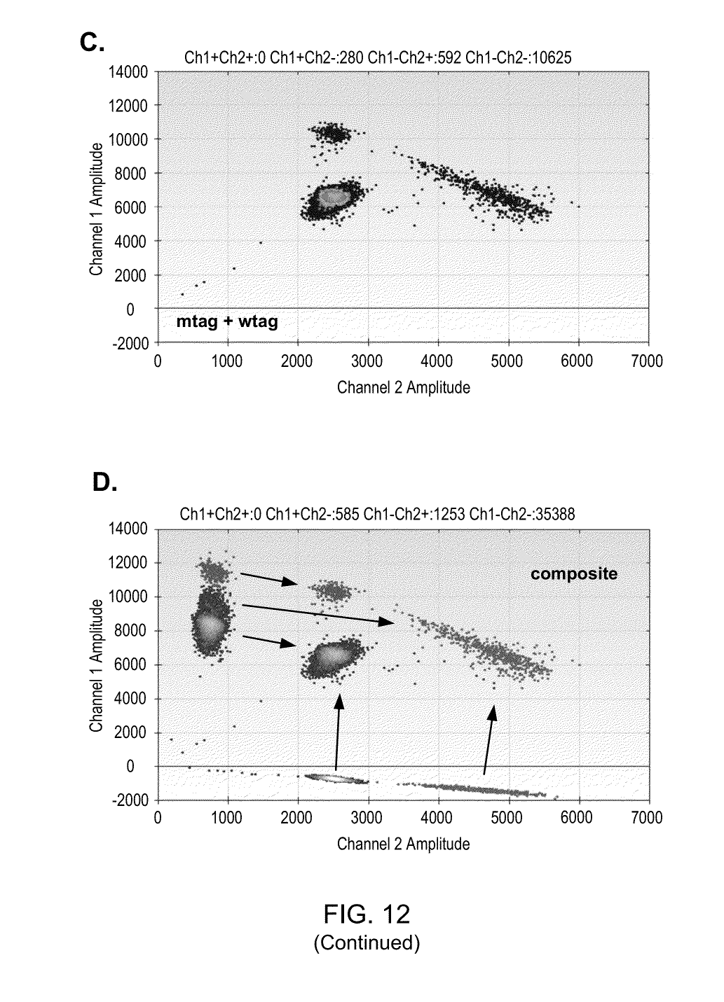

Genomic DNA from NA18507 cell lines was used as a surrogate for wild-type DNA (wt) and obtained as purified stocks (Coriell). Two microliters of a mixture containing wt (30 ng) and mu (6 ng) DNA was assembled into a 20 μl ddPCR reaction mix from 2×ddPCR supermix for probes, 0.2 uM final of each forward primer (wt: 5′-AGATTACGCGGCAATAAGGCTCGGTTGGCATTGGATACTACTTGCCTACGCCACC-3′; mu: 5′AATAGCTGCCTACATTGGGTTCGGTCGTAACTTAGGAACTCTTGCCTACGCCAGC-3′), 0.4 uM of reverse primer (5′-CCTGCTGAAaAATGACTGAAT-3′), and 1 uM each of reporter probes (wt: 5′-HEX-CCAACCGAG/ZEN/CCTTATTGCCG-IABkFQ-3′; mu: 5′-FAM-AGTTACGAC/ZEN/CGAACCCAATGTAGG-IABkFQ-3′). Each PCR mixture was then converted into droplets for analysis via the QX100 ddPCR system according to the manufacturer's suggestions. Annealing temperature was varied to determine the optimal conditions for segregating and quantifying the wt (HEX) and mu (FAM) droplet signals (FIG. 11). Resulting clusters were deconvoluted (FIG. 12) by using ddPCR mixtures containing only the mu (12A), only the wt (12B), or both probes (12C) to assign membership of each cluster as mu or wt.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of monitoring cancer progression in a subject in need thereof, comprising:
(a) determining the presence, absence, and/or amount of each of a subset of genes in a sample derived from a fluid sample in the subject, wherein the subset is determined by (i) performing targeted sequencing on a set of genes on a solid tissue sample from the subject wherein the solid tissue sample is known or suspected of comprising cancerous tissue; (ii) determining a profile of genetic mutations for said set of genes based on the sequencing; and (iii) selecting a subset of 2, 3, or 4 genes of the set of genes based on said profile for said set, wherein said subset is specific to said subject; and
(b) using the results of (a) to monitor progression of the cancer or lack thereof in the subject.

2. (canceled)

3. The method of claim 1, wherein said set of genes comprises at least 10 genes.

4. (canceled)

5. (canceled)

6. The method of claim 1, wherein the set of genes is selected from the group consisting of ABCA1, BRAF, CHD5, EP300, FLT1, ITPA, MYC, PIK3R1, SKP2, TP53, ABCA7, BRCA1, CHEK1, EPHA3, FLT3, JAK1, MYCL1, PIK3R2, SLC19A1, TP73, ABCB1, BRCA2, CHEK2, EPHA5, FLT4, JAK2, MYCN, PKHD1, SLC1A6, TPM3, ABCC2, BRIP1, CLTC, EPHA6, FN1, JAK3, MYH2, PLCB1, SLC22A2, TPMT, ABCC3, BUB1B, COL1A1, EPHA7, FOS, JUN, MYH9, PLCG1, SLCO1B3, TPO, ABCC4, Clorf144, COPS5, EPHA8, FOXO1, KBTBD11, NAV3, PLCG2, SMAD2, TPR, ABCG2, CABLES1, CREB1, EPHB1, FOXO3, KDM6A, NBN, PML, SMAD3, TR10, ABL1, CACNA2D1, CREBBP, EPHB4, FOXP4, KDR, NCOA2, PMS2, SMAD4, TRRAP, ABL2, CAMKV, CRKL, EPHB6, GAB1, KIT, NEK11, PPARG, SMARCA4, TSC1, ACVR1B, CARD11, CRLF2, EPO, GATA1, KLF6, NF1, PPARGC1A, SMARCB1, TSC2, ACVR2A, CARM1, CSF1R, ERBB2, GLI1, KLHDC4, NF2, PPP1R3A, SMO, TTK, ADCY9, CAV1, CSMD3, ERBB3, GLI3, KRAS, NKX2-1, PPP2R1A, SOCS1, TYK2, AGAP2, CBFA2T3, CSNK1G2, ERBB4, GNA11, LMO2, NOS2, PPP2R1B, SOD2, TYMS, AKT1, CBL, CTNNA1, ERCC1, GNAQ, LRP1B, NOS3, PRKAA2, SOS1, UGT1A1, AKT2, CCND1, CTNNA2, ERCC2, GNAS, LRP2, NOTCH1, PRKCA, SOX10, UMPS, AKT3, CCND2, CTNNB1, ERCC3, GPR124, LRP6, NOTCH2, PRKCZ, SOX2, USP9X, ALK, CCND3, CYFIP1, ERCC4, GPR133, LTK, NOTCH3, PRKDC, SP1, VEGF, ANAPC5, CCNE1, CYLD, ERCC5, GRB2, MAN1B1, NPM1, PTCH1, SPRY2, VEGFA, APC, CD40LG, CYP19A1, ERCC6, GSK3B, MAP2K1, NQO1, PTCH2, SRC, VHL, APC2, CD44, CYP1B1, ERG, GSTP1, MAP2K2, NR3Cl, PTEN, ST6GAL2, WRN, AR, CD79A, CYP2C19, ERN2, GUCY1A2, MAP2K4, NRAS, PTGS2, STAT1, WT1, ARAF, CD79B, CYP2C8, ESR1, HDAC1, MAP2K7, NRP2, PTPN11, STAT3, XPA, ARFRP1, CDC42, CYP2D6, ESR2, HDAC2, MAP3K1, NTRK1, PTPRB, STK11, XPC, ARID1A, CDC42BPB, CYP3A4, ETV4, HGF, MAPK1, NTRK2, PTPRD, SUFU, ZFY, ATM, CDC73, CYP3A5, EWSR1, HIF1A, MAPK3, NTRK3, RAD50, SULT1A1, ZNF521, ATP5A1, CDH1, DACH2, EXT1, HM13, MAPK8, OMA1, RAD51, SUZ12, ATR, CDH10, DCC, EZH2, HMGA1, MARK3, OR10R2, RAFT, TAF1, AURKA, CDH2, DCLK3, FANCA, HNF1A, MCL1, PAK3, RARA, TBX22, AURKB, CDH2O, DDB2, FANCD2, HOXA3, MDM2, PARP1, RB1, TCF12, BAI3, CDH5, DDB2, FANCE, HOXA9, MDM4, PAX5, REM1, TCF3, BAP1, CDK2, DGKB, FANCF, HRAS, MECOM, PCDH15, RET, TCF4, BARD1, CDK4, DGKZ, FAS, HSP90AA1, MEN1, PCDH18, RICTOR, TEK, BAX, CDK6, DIRAS3, FBXW7, IDH1, MET, PCNA, RIPK1, TEP1, BCL11A, CDK7, DLG3, FCGR3A, IDH2, MITF, PDGFA, ROR1, TERT, BCL2, CDK8, DLL1, FES, IFNG, MLH1, PDGFB, ROR2, TET2, BCL2A1, CDKN1A, DNMT1, FGFR1, IGF1R, MLL, PDGFRA, ROS1, TGFBR2, BCL2L1, CDKN1B, DNMT3A, FGFR2, IGF2R, MLL3, PDGFRB, RPS6KA2, THBS1, BCL2L2, CDKN2A, DNMT3B, FGFR3, IKBKE, MPL, PDZRN3, RPTOR, TNFAIP3, BCL3, CDKN2B, DOT1L, FGFR4, IKZF1, MRE11A, PHLPP2, RSPO2, TNKS, BCL6, CDKN2C, DPYD, FH, IL2RG, MSH2, PIK3C3, RSPO3, TNKS2, BCR, CDKN2D, E2F1, FHOD3, INHBA, MSH6, PIK3CA, RUNX1, TNNI3K, BIRC5, CDX2, EED, FIGF, INSR, MTHFR, PIK3CB, SDHB, TNR, BIRC6, CEBPA, EGF, FLG2, IRS1, MTOR, PIK3CD, SF3B1, TOP1, BLM, CERK, EGFR, FLNC, IRS2, MUTYH, PIK3CG, SHC1, and TOP2A.

7. The method of claim 1, wherein the fluid sample is selected from the group consisting of blood, serum, plasma, urine, sweat, tears, saliva, or sputum.

8. The method of claim 1, wherein (a) and (b) are performed at a plurality of time points to monitor the status of the cancer over time.

9. (canceled)

10. The method of claim 1, further comprising generating a report communicating the profile of genetic mutations for the set of genes and communicating said report to a caregiver.

11. (canceled)

12. (canceled)

13. The method of claim 10, wherein the report comprises copy number alterations of said set of genes.

14. (canceled)

15. (canceled)

16. The method of claim 1, wherein said determining comprises diluting said nucleic acid molecules from said sample into discrete reaction volumes, wherein said discrete reaction volumes contain between 1 to 10 molecules of said nucleic acid molecule from said sample.

17. The method of claim 16, wherein said discrete reaction volumes are droplets in an emulsion.

18. (canceled)

19. (canceled)

20. The method of claim 1, wherein the targeted sequencing comprises preparing a DNA library from said solid tissue sample, wherein said preparing does not require exponential PCR amplification prior to sequencing of said library.

21. (canceled)

22. (canceled)

23. The method of claim 1, wherein the step of targeted sequencing comprises:
(a) contacting a single-stranded DNA fragment from said solid tissue sample with a target-specific oligonucleotide comprising (i) a region specific for a region of a cancer-related gene and (ii) an adaptor sequence specific for coupling to a sequencing platform;
(b) performing a hybridization reaction to join said target specific oligonucleotides to a single-stranded DNA fragment containing a region of complementarity to said target-specific oligonucleotide;
(c) performing an extension reaction to create an extension product comprising said region and comprising said adaptor; and
(d) sequencing said extension product.

24. (canceled)

25. The method of claim 23, wherein said contacting occurs with the target-specific oligonucleotide free in a solution.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. A method of detecting a mutation in a target polynucleotide region, comprising:
(a) selectively hybridizing an oligonucleotide primer to said target polynucleotide region, wherein said oligonucleotide primer comprises (i) a probe-binding region, and (ii) a template binding region that is at least 70% complementary to a template nucleic acid suspected of harboring a mutation, wherein a portion of said template binding region at least partially overlays a locus of said suspected mutation, and wherein said oligonucleotide primer upon hybridization to said template nucleic acid is extendable by a polymerase if said mutation is present but is not extendable by said polymerase if said mutation is not present;
(b) extending said hybridized oligonucleotide primer to form an extension product; and
(c) detecting said extension product, whereby wherein said detecting indicates the presence of said mutation.

49. The method of claim 48, wherein said extending comprises extending with a DNA polymerase that does not comprise 3′ to 5′ exonuclease activity.

50. The method of claim 48, wherein said detecting comprises selectively hybridizing a reporter probe to said probe binding region.

51. The method of claim 50, wherein said reporter probe comprises a detectable moiety and a quencher moiety, wherein said quencher moiety suppresses detection of said detectable moiety when said reporter probe is intact.

52. (canceled)

53. The method of claim 50, further comprising amplifying said extension product with a reverse primer that is capable of hybridizing to a region of said extension product downstream of said locus.

54. The method of claim 53, wherein said amplifying comprises amplifying with a DNA polymerase that comprises 5′ to 3′ exonuclease activity.

55. (canceled)

56. (canceled)

57. (canceled)

58. (canceled)

59. (canceled)

60. The method of claim 48, wherein said mutation is a single nucleotide polymorphism (SNP).

61. The method of claim 60, wherein said template binding region comprises a 3′ terminal region having a base that overlays said SNP locus.

62. (canceled)

63. (canceled)

64. The method of claim 48, wherein said probe-binding region does not hybridize to said target polynucleotide region.

65. The method of claim 48, further comprising subdividing said nucleic acid sample into a plurality of discrete reaction volumes and detecting a presence or absence of said detectable moiety in each of said reaction volumes.

66. The method of claim 65, further comprising counting a number of said reaction volumes wherein said detectable moiety is detected.

67. (canceled)

68. (canceled)

69. (canceled)

70. A method, comprising:
(a) hybridizing a target-selective oligonucleotide (TSO) to a single-stranded DNA (ssDNA) fragment of an ssDNA library to create a hybridization product, wherein the TSO comprises (i) a region sequence that selectively is complementary to a single target genomic region and (ii) a second single-stranded adaptor sequence located at a first end of the TSO, wherein the ssDNA fragment comprises a second adaptor sequence attached to said ssDNA fragment via single-stranded ligation and further wherein said second adaptor sequence is different than said first adaptor sequence; and
(b) amplifying the hybridization product to create a double stranded extension product.

71. The method of claim 1, wherein the fluid sample is a cell free or substantially cell free genomic sample.

72. The method of claim 1, wherein the monitoring of the cancer progression is used to monitor the efficacy of a cancer treatment.

73. The method of claim 1, wherein the profile of genetic mutations comprises copy number variations.

74. The method of claim 1, wherein the one or more genes comprises TP53.

75. The method of claim 74, wherein the cancer is breast cancer.

76. A method for assessing status of cancer in a subject, comprising:
(a) obtaining a cell free plasma sample from said subject;
(b) extracting targeted polynucleotides from said sample;
(c) analyzing by digital sequencing said targeted polynucleotides to obtain a genetic profile for a set of genes; and,
(d) using said genetic profile to assess the status of the cancer in said subject.

77. The method of claim 76, wherein the set of genes is determined by (i) performing targeted sequencing on a plurality of genes using a tissue sample from the subject wherein the tissue sample is known or suspected of comprising cancerous tissue; (ii) detecting genetic mutations for the plurality of genes based on the sequencing; and (iii) correlating the genetic mutations to identify the set of genes associated with the cancer in said subject.

78. The method of claim 51, wherein a hybridization product of said oligonucleotide primer and reporter probe has a Tm that is at least 10 degrees higher than a Tm of a hybridization product between said oligonucleotide primer and target polynucleotide.
